(19) United States
(12) Patent Application Publication
(10) Pub. No.: US 2008/0286325 A1
(43) Pub. Date: Nov. 20, 2008

(54) CYCLODEXTRIN ELUTION MEDIA FOR MEDICAL DEVICE COATINGS COMPRISING A TAXANE THERAPEUTIC AGENT

(75) Inventors: Priscilla Reyes, West Lafayette, IN (US); Patrick H. Ruane, Redwood City, CA (US); Melinda S. Morrell, Vienna, VA (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: MED Institute, Inc., West Lafayette, IN (US)

(21) Appl. No.: 11/774,322

(22) Filed: Jul. 6, 2007

Related U.S. Application Data
(63) Continuation-in-part of application No. 11/715,975, filed on Mar. 8, 2007.
Application No. 11/715,975 is a continuation-in-part of application No. 11/650,034, filed on Jan. 5, 2007.
(60) Provisional application No. 60/781,264, filed on Mar. 10, 2006.
(60) Provisional application No. 60/830,726, filed on Jul. 13, 2006.
(60) Provisional application No. 60/830,660, filed on Jul. 13, 2006.
(60) Provisional application No. 60/818,175, filed on Jun. 30, 2006.
(60) Provisional application No. 60/756,451, filed on Jan. 5, 2006.

(51) Int. Cl. A61K 9/00; A61K 31/337; G01N 33/15

(52) U.S. Cl. 424/423; 514/449; 436/93

(57) ABSTRACT

The present disclosure provides methods of measuring the release of a taxane therapeutic agent from a medical device as a function time in contact with a suitable elution medium. The method preferably comprises the step of contacting a coated medical device comprising a taxane therapeutic agent with an elution medium comprising a cyclodextrin to provide an elution profile indicative of the composition or configuration of a medical device coating comprising a taxane therapeutic agent. The elution profile can provide information about the medical device coating that is useful in lot release testing.

### RELATED APPLICATIONS

This application is a continuation-in-part of the following co-pending U.S. patent application Ser. No. ______, entitled “Methods of Manufacturing and Modifying Taxane Coatings for Implantable Medical Devices” and filed Jun. 27, 2007 by Reyes et al.; Ser. No. 11/715,975 filed Mar. 8, 2007; and Ser. No. 11/650,034, filed Jan. 5, 2007. Based on U.S. patent application Ser. No. 11/715,975, this application claims the benefit of the following U.S. Provisional Patent Application Ser. No. 60/781,264, entitled “Taxane Coatings for Implantable Medical Devices” and filed Mar. 10, 2006; Ser. No. 60/830,726, entitled “Controlled Release Taxane Coatings for Implantable Medical Devices” and filed Jul. 13, 2006; and Ser. No. 60/830,660, entitled “Cyclodextrin Elution Media for Medical Device Coatings Comprising a Taxane Therapeutic Agent” and filed Jul. 13, 2006. Based on co-pending U.S. patent application Ser. No. ______, entitled “Methods of Manufacturing and Modifying Taxane Coatings for Implantable Medical Devices” and filed Jun. 27, 2007, this application claims the benefit of the following U.S. Provisional Patent Application Ser. No. 60/781,264, entitled “Taxane Coatings for Implantable Medical Devices” and filed Mar. 10, 2006; Ser. No. 60/818,175, entitled “Methods of Manufacturing Taxane Coatings for Endolumenal Medical Devices,” and filed Jun. 30, 2006; Ser. No. 60/830,726, entitled “Controlled Release Taxane Coatings for Implantable Medical devices” and filed Jul. 13, 2006; and Ser. No. 60/830,660, entitled “Cyclodextrin Elution Media for Medical Device Coatings Comprising a Taxane Therapeutic Agent” and filed Jul. 13, 2006. Based on U.S. patent application Ser. No. 11/650,034, filed Jan. 5, 2007, this application also claims the benefit of U.S. provisional patent application Ser. No. 60/756,451, filed Jan. 5, 2006. Each of the above-referenced patent applications is incorporated herein by reference in their entirety.

### TECHNICAL FIELD

The present disclosure relates to releasable taxane therapeutic agent coatings for endolumenal medical devices, including stents. In particular, the disclosure provides methods for detecting the release of taxane therapeutic agents from medical devices in elution media comprising a cyclodextrin. Lot release testing methods for medical devices comprising a taxane therapeutic agent are also described.

### BACKGROUND

Lot release testing is one of the methods used by regulatory agencies, such as the U.S. Food and Drug Administration (“FDA”) to ensure that implantable products, such as drug eluting medical devices, are safe and have been manufactured in accordance with laws and regulations. The FDA, or other regulatory agencies, may require lot samples and protocols showing results of applicable tests to be submitted for review and possible testing by FDA.

For most implantable products, each product lot may undergo thorough testing by a manufacturer for purity, potency, identity, and sterility. The lot release program is a risk prevention measure that provides a quality control check on product specifications and also provides samples and documentation to permit follow-up investigations if safety issues arise. Numerous lots are submitted for release each year and manufacturers often release lots only after this testing is documented. Each lot of product may be released for its intended use if it meets prospectively defined quality control criteria. Lots may be controlled at various points in the production process, including during manufacturing, in bulk forms, or as final products. For example, products may be controlled for identity, purity, potency, sterility (parenteral products) or bioburden (non-parenteral products), effectiveness and safety. Lot release documentation may include the COA and the raw data or data worksheets for in-process, bulk, and final product testing.

Implantable medical devices, such as an endolumenal stent or valve, can be adapted to release a coated therapeutic agent to treat or mitigate undesirable conditions including restenosis, tumor formation or thrombosis. Procedures for mitigating certain conditions can include implantation of a device comprising a therapeutic agent. For example, the implantation of stents during angioplasty procedures has substantially advanced the treatment of occluded body vessels. Angioplasty procedures such as Percutaneous Transluminal Coronary Angioplasty (PTCA) can widen a narrowing or occlusion of a blood vessel by dilation with a balloon. Occasionally, angioplasty may be followed by an abrupt closure of the vessel or by a more gradual closure of the vessel, commonly known as restenosis. Acute closure may result from an elastic rebound of the vessel wall and/or by the deposition of blood platelets and fibrin along a damaged length of the newly opened blood vessel. In addition, restenosis may result from the natural healing reaction to the injury to the vessel wall (known as intimal hyperplasia), which can involve the migration and proliferation of medial smooth muscle cells that continues until the vessel is again occluded. To prevent such vessel occlusion, stents have been implanted within a body vessel. However, restenosis may still occur over the length of the stent and/or past the ends of the stent where the inward forces of the stenosis are unopposed. To reduce incidence of restenosis, one or more therapeutic agents may be coated on an implantable stent for release within the body vessel after implantation.

For medical devices coated with a releasable therapeutic agent, such as drug eluting stents, the FDA may require lot testing including a drug elution profile showing the rate of release of a therapeutic agent from the coated medical device as a function of time in a suitable elution medium, such as porcine serum. There is a need for intravascularly-implantable medical devices capable of releasing a therapeutic agent at a desired rate and over a desired time period upon implantation. Preferably, an implanted medical device releases a therapeutic agent at the site of medical intervention to promote a therapeutically desirable outcome, such as mitigation of restenosis. Accordingly, methods of measuring the rate of release of the therapeutic agent from the coated medical device are useful in performing lot release testing on the coated medical devices. In particular, there is a need for methods for measuring the release of a taxane therapeutic agent from an implantable medical device.

Taxane therapeutic agents can be used as a therapeutic agent coated on and released from implantable devices, such as stents, to mitigate or prevent restenosis. Taxane therapeutic agents, including paclitaxel and taxane analogues and derivatives thereof, are believed to disrupt mitosis (M-phase) by binding to tubulin to form abnormal mitotic spindles or an analogue or derivative thereof. Coatings of taxane therapeutic agents can include various crystalline species having a different arrangement of the taxane molecules in the solid. For example, paclitaxel and taxane derivatives thereof can be formed in three different solid forms of paclitaxel at room temperature, which have been identified as amorphous paclitaxel (“aPTX”), dihydrate crystalline paclitaxel (“dPTX”) and anhydrous paclitaxel. Different solid forms of paclitaxel can be characterized and identified using various solid-state analytical tools, for example as described by Jeong Hoon Lee et al., “Preparation and Characterization of Solvent Induced Dihydrated, Anhydrous and Amorphous Paclitaxel,” Bull. Korean Chem. Soc. v. 22, no. 8, pp. 925-928 (2001), incorporated herein by reference. Taxane therapeutic agent in the different solid forms can have different solubilities, which can lead to different rates of elution upon implantation within a body vessel. U.S. Pat. No. 6,858,644, filed Nov. 26, 2002 by Benigni et al., (“Benigni”), teaches a crystalline solvate comprising paclitaxel and a solvent selected from the group consisting of dimethylsulfoxide, N,N′-dimethylformamide, N,N′-dimethylacetamide, N-methyl-2-pyrrolidone, 1,3-dimethyl-2-imidazolidinone, 1,3-dimethyl-3,4,5,6-tetrahydro-2-(1H)-pyrimidinone, and acetonitrile and combinations thereof. However, Benigni does not describe implantable device coatings comprising crystalline paclitaxel forms with different elution rates. Benigni discloses various solid forms of paclitaxel, including a first solid form reported as a highly water insoluble crystalline, granular, solvent-free form. The first solid form is substantially non-hygroscopic under normal laboratory conditions (relative humidity (RH) approximately 50-60%; 20-30° C.). However, when contacted with an atmosphere having a relative humidity greater than about 90%, or in aqueous suspensions, dispersions or emulsions, the first paclitaxel solid form reportedly converts (as a function of time, temperature, agitation, etc.) to a thermodynamically more stable second solid form. The second solid form is described as a trihydrate orthorhombic form having six water sites per two independent paclitaxel molecules (one paclitaxel “dimer”). These hydrated crystals reportedly present a fine, hair-like appearance and are even less water soluble than the first solid form. The second solid form is reportedly formed in aqueous suspensions or through crystallization from aqueous solvents in the presence of a large excess of water. This form is also disclosed in patent application EP 0 717 041, which describes the second solid form as being characterized by single crystal X-ray diffraction studies as being orthorhombic, with unit cells containing two crystallographically independent molecules of paclitaxel associated with hydrogen bonds to form a “dimer”. Mastropaolo, et al. disclosed a crystalline solvate of paclitaxel obtained by evaporation of solvent from a solution of Taxol® in dioxane, water and xylene. Proc. Natl. Acad. Sci. USA 92, 6920-24 (July, 1995). This solvate is indicated as being unstable, and, in any event, has not been shown to effect purification of crude paclitaxel. The thin plate-like crystals are reported to contain five water molecules and three dioxane molecules per two molecules of paclitaxel. None of these references describe a durable taxane coating having an elution profile that can be altered by treatment of a medical device coating to vary the solid form composition of the coating.

Often, coatings combine a releasable taxane therapeutic agent with one or more materials to modify the rate of release of the taxane therapeutic agent from the medical device upon implantation. These release modifying agents are often polymers, such as biodegradable or porous biostable polymers that are mixed with or coated over the taxane therapeutic agent. The rate of release of the taxane therapeutic agent from a medical device coating may depend on the solid form of the taxane therapeutic agent, the addition of a release modifying agent to the coating, and the coating configuration.

A lot release method can include measurement of the rate of release of a taxane therapeutic agent by contacting the coated medical device with porcine serum and measuring the rate of elution of the taxane therapeutic agent into the porcine serum. However, the taxane therapeutic agent may require extended periods of time to elute in porcine serum, often on the order of 3 days to 30 days or longer, depending on the configuration of the coating. Such extended elution times may add to the time and expense of obtaining elution profile data for lot release testing. Alternatively, the taxane therapeutic agent may be very rapidly dissolved in another elution medium, such as sodium dodecyl sulfate (SDS), often in less than about one hour. However, while different crystalline forms of a taxane therapeutic agent may dissolve at different rates upon implantation in a blood vessel or in porcine serum, the rates of dissolution of both solid forms of the taxane therapeutic agent in SDS are typically so rapid as to be difficult to distinguish. Similarly, medical device coatings comprising differing amounts of a bioabsorbable polymer such as poly(lactic acid) (PLA) and a taxane therapeutic agent, in the same or separate layers, may dissolve at different rates upon implantation in a body vessel or in porcine serum, but indistinguishably rapidly in SDS.

What is needed are methods of obtaining elution profile data for the elution of taxane therapeutic agents from coated medical devices in a manner that permits measurement of relative solubility rates of different coating configurations, such as coatings comprising a taxane therapeutic agent in one or more solid crystalline forms, or coatings comprising a bioabsorbable polymer in combination with the taxane therapeutic agent. There is also a need for methods of detecting the amount of therapeutic agent in a coating, and the configuration of the coating, in a desirably short time period. For example, many existing lot release protocols require solubility testing of therapeutic agent coatings over undesirably long periods of time to determine the elution profile of the therapeutic agent. What is needed are methods for performing such lot release tests in desirably shorter time periods in a manner that permits identification of both the total amount of therapeutic agent and elution profiles indicative of different coating configurations.

### SUMMARY

The present disclosure provides a method of identifying and/or distinguishing different compositions or configurations of medical device coatings comprising a taxane therapeutic agent by measuring the elution profile of the coating in a suitable elution medium. For example, methods are provided for determining the total amount of a taxane therapeutic agent (e.g., paclitaxel) in a coated medical device, as well as determining the configuration or composition the coating, by contacting the coated medical device with an elution medium comprising a cyclodextrin to obtain an elution profile. The use of a cyclodextrin-containing elution medium may provide an elution profile useful for lot release testing in a considerably shorter period of time (e.g., at least about 10-times shorter) compared to the use of a porcine serum elution medium, while still being able to differentiate between different coating compositions on the basis of the elution profile.

An elution profile is a graph recording the amount of the taxane therapeutic agent released (the elution rate) from a coated medical device as a function of the duration of contact between the elution medium and the medical device coating. Differences between medical device coatings comprising a taxane therapeutic agent that lead to distinguishable elution profiles can be probed by measuring the elution profile of the coating in a suitable elution medium. Preferably, elution media can be selected to provide taxane therapeutic agent elution rates that are desirably rapid enough to record an elution profile over a desirably short period of time, while simultaneously providing an elution profile that remains dependent on, and/or indicative of, the structure or composition of the taxane therapeutic agent in the coating. Accordingly, elution profiles of medical device coatings are useful in providing lot release data relating to the composition of taxane-coated medical devices, including paclitaxel-coated stents.

The method preferably comprises the step of contacting a coated medical device comprising a taxane therapeutic agent with an elution medium comprising a cyclodextrin. A cyclodextrin is a cyclic oligosaccharide formed from covalently-linked glucopyranose rings defining an internal cavity. The diameter of the internal axial cavity of cyclodextrins increases with the number of glucopyranose units in the ring. The size of the glucopyranose ring can be selected to provide an axial cavity selected to match the molecular dimensions of a taxane therapeutic agent. The cyclodextrin is preferably a modified β-cyclodextrin, such as Heptakis-(2,6-di-O-methyl)-β-cyclodextrin (HCD). Suitable cyclodedtrin molecules include other β-cyclodextrin molecules, as well as γ-cyclodextrin structures.

Obtaining an elution profile by contacting a taxane-coated medical device with an elution medium comprising a suitable cyclodextrin provides a method for obtaining lot release data indicative of differences in coating configuration that are distinguishable based on solubility of the taxane therapeutic agent in the cyclodextrin. The elution medium comprising a cyclodextrin can dissolve a taxane therapeutic agent so as to elute the taxane therapeutic agent from a medical device coating over a desired time interval, typically about 24 hours or less. Preferably, the cyclodextrin elution medium is formulated to provide distinguishable elution rates for different coating configurations, such as different solid forms of a taxane therapeutic agent in the coating, or different types or amounts of polymers incorporated with the taxane therapeutic agent within a coating. The elution medium may be contacted with a medical device comprising a taxane therapeutic agent, such as paclitaxel, in any manner providing an elution profile indicative of the arrangement of the taxane therapeutic agent molecules in the coating. For example, the elution medium may contact a medical device coating in a continuous flow configuration, or in a batch testing configuration, as discussed below. The elution profile of a medical device coating formed from a solvated solid form of a taxane therapeutic agent measured in a cyclodextrin elution medium typically provides a distinguishably slower rate of elution than a medical device coating formed from an amorphous solid form of the taxane therapeutic agent in the same elution medium. Similarly, the elution profile of a coating comprising both a taxane therapeutic agent and differing amounts of a biodegradable elastomer, such as poly(lactic acid), can be distinguished based on the elution profiles in a cyclodextrin elution medium.

Optionally, the methods disclosed for lot release testing may include preparation of one or more standard coated medical devices with known coating compositions or configurations, obtaining an elution profile from each standard coated medical device, and comparing these elution profiles with the elution profile(s) obtained from one or more coated medical devices having an unknown composition and/or configuration.

Methods of detecting taxane therapeutic agents using cyclodextrin elution media offer multiple advantages for lot release testing application. First, elution profiles of medical device coatings comprising a polymer and a taxane therapeutic agent obtained in cyclodextrin elution media can distinguish between different coating configurations, such as different amounts of a biodegradable polymer present in the coating. Second, cyclodextrin elution media typically elute in a considerably shorter time period than that required for comparable elution in porcine serum. Information about a medical device coating, such as the solid form of the taxane therapeutic agent or the amount of polymer in the coating, can be evaluated by comparing a first elution profile obtained from a coated stent in a cyclodextrin elution medium, and comparing the elution profile with a second elution profile obtained from a standard coated stent having a known composition obtained in the cyclodextrin elution medium. The degree to which the first elution profile is similar to the second elution profile, or any portion thereof, can be used as a lot release testing criteria to evaluate the quality of the coated stent.

### BRIEF DESCRIPTION OF THE DRAWINGS

FIG. 1A is a schematic of certain preferred methods of detecting a taxane therapeutic agent.

FIG. 1B is the molecular structure of paclitaxel.

FIG. 1C is a molecular structure formula of certain cyclodextrin molecules.

FIG. 2 shows an ultraviolet (UV) absorption spectrum of paclitaxel in ethanol.

FIG. 3A shows an infrared spectrum of a first solid form of paclitaxel.

FIG. 3B shows an infrared spectrum of a second solid form of paclitaxel.

FIG. 3C shows an infrared spectrum of a third solid form of paclitaxel.

FIG. 4A shows a series of confocal Raman spectra for various solid forms paclitaxel.

FIG. 4B shows the spatial distribution of two different solid forms of paclitaxel as a function of coating depth, obtained using confocal Raman spectroscopy.

FIG. 5A shows a powder X-ray diffraction (XRPD) spectrum of two different solid forms of paclitaxel.

FIG. 5B shows a ¹³C NMR spectrum of three different solid forms of paclitaxel.

FIG. 6A shows elution profiles for coatings of amorphous paclitaxel and solvated paclitaxel eluting in porcine serum.

FIG. 6B shows elution profiles for coatings each comprising different amounts of the amorphous and dihydrate solid forms of paclitaxel eluting in an aqueous solution of Heptakis-2,6-di-O-methyl)-β-cyclodextrin (HCD).

FIG. 6C shows elution profiles for several different coatings having different amounts of the amorphous and dihydrate solid forms of paclitaxel eluting in porcine serum.

FIG. 7A shows an elution profile for a coating of the amorphous solid form of paclitaxel eluting in an aqueous solution of sodium dodecyl sulfate (SDS).

FIG. 7B shows the elution profile for a coating of the dihydrate solid form of paclitaxel eluting in an aqueous solution of SDS.

FIG. 8A is a kinetic plot for the dissolution of amorphous paclitaxel in porcine serum.

FIG. 8B is a kinetic plot for the dissolution of dihydrate paclitaxel in porcine serum.

FIG. 9 is a graph of calculated (predicted) porcine serum solubility of a paclitaxel coating comprising varying amounts of the dihydrate paclitaxel and the amorphous paclitaxel in varying proportions.

FIG. 10A and FIG. 10B are optical micrographs of a paclitaxel coated stent.

FIG. 11A and FIG. 11B are optical micrographs of a paclitaxel coated stent.

FIG. 12A and FIG. 12B are optical micrographs of a paclitaxel coated stent.

FIG. 13A and FIG. 13B are optical micrographs of a paclitaxel coated stent.

FIG. 14 is a graph showing the elution profiles of two different paclitaxel coated stents in an aqueous solution of HCD.

FIG. 15 is a graph showing the elution profiles from two different paclitaxel coated stents in an aqueous solution of HCD.

FIG. 16 shows a coated endolumenal medical device.

FIG. 17A shows a cross sectional view of a portion of the medical device of FIG. 16.

FIG. 17B shows an alternative cross-sectional view of the portion of the medical device of FIG. 16.

FIG. 18A is a schematic of a batch apparatus for detecting a taxane therapeutic agent eluted from a coated medical device.

FIG. 18B is a schematic of a flow-through apparatus for detecting a taxane therapeutic agent eluted from a coated medical device.

FIG. 19A shows the elution profile of amorphous paclitaxel in a 0.5% aqueous solution of Heptakis-(2,6-di-O-methyl)-β-cyclodextrin (HCD).

FIG. 19B shows the elution profile of amorphous paclitaxel in a 0.2% aqueous solution of Heptakis-(2,6-di-O-methyl-β-cyclodextrin (HCD).

FIG. 19C shows the elution profile of coating comprising a first mixture of dihydrate paclitaxel and amorphous paclitaxel in a 0.5% aqueous solution of Heptakis-(2,6-di-O-methyl)-β-cyclodextrin (HCD), followed by elution in a 0.5% aqueous solution of sodium dodecyl sulfate (SDS).

FIG. 19D shows the elution profile of coating comprising a second mixture of dihydrate paclitaxel and amorphous paclitaxel in a 0.5% aqueous solution of Heptakis-(2,6-di-O-methyl)-β-cyclodextrin (HCD), followed by elution in a 0.5% aqueous solution of sodium dodecyl sulfate (SDS).

FIG. 20 shows two elution profiles of a two-layer coated medical device comprising a layer of paclitaxel covered by a layer of poly(lactic acid) (PLA). The first elution profile was obtained in a 5% aqueous solution of Heptakis-(2,6-di-O-methyl)-β-cyclodextrin (HCD), and the second elution profile was obtained in porcine serum.

FIG. 21 shows three elution profiles of a two-layer coated medical device comprising a layer of paclitaxel covered by a second layer comprising different amounts of poly(lactic acid) (PLA). Each elution profile was obtained in a 5% aqueous solution of HCD.

FIG. 22 shows an elution profile of a two-layer coated medical device comprising a layer of paclitaxel covered by a second layer comprising zein. The elution profile was obtained in a 5% aqueous solution of HCD.

FIG. 23 shows three elution profiles of a two-layer coated medical device comprising a layer of paclitaxel covered by a second layer comprising different amounts of PLA or zein. Each elution profile was obtained in a 5% aqueous solution of HCD.

### DETAILED DESCRIPTION

The present disclosure provides lot release testing methods comprising the step of measuring the release of a taxane therapeutic agent from a medical device as a function of time that the coating is in contact with a suitable elution medium. The method preferably comprises the step of contacting a coated medical device comprising a taxane therapeutic agent with an elution medium comprising a cyclodextrin to provide an elution profile indicative of the composition or configuration of the medical device coating. The elution profile can provide information about the medical device coating that is useful in lot release testing.

Unless otherwise specified, description of paclitaxel coatings herein relate to a preferred embodiment of the taxane therapeutic agent, and is intended to be illustrative of all taxane therapeutic agents capable of forming two or more of the solid forms described, without limiting the scope of the therapeutic agent to paclitaxel. For example, a first elution profile can be obtained from a first paclitaxel-coated stent in a cyclodextrin elution medium. The first coated stent can be a representative test sample selected from a group of coated stents. The elution properties and solid form of the paclitaxel in the first stent may be unknown. The first elution profile can be compared with a second elution profile obtained from a standard coated stent having a known paclitaxel structure and composition, as well as a desirable elution profile obtained in the cyclodextrin elution medium. The structure of the standard coated stent can be verified by various characteristics in addition to its elution profile, such as Raman vibrational spectroscopy and melting point. The degree to which the first elution profile is similar to the second elution profile, or any portion thereof, can then be used as a lot release testing criteria to evaluate the quality of the first coated stent. Analysis of the elution profiles of medical device coatings can be used to distinguish between different coating configurations in a desirably shorter time period than that required by many existing elution testing methods, such as measuring elution into porcine serum. The methods provided herein permit measuring the elution profile of coated medical devices comprising a taxane therapeutic agent in a desirably short period of time in a manner permitting identification of relevant structural or compositional changes in a coating (i.e., any change in the coating that can be correlated to a change in the elution profile). Therefore, the methods of detecting and measuring the release of a taxane therapeutic agent into an elution medium comprising a cyclodextrin are particularly advantageous for providing lot release test data.

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

The terms “absorption,” “bioresorption” and “bioabsorption” can be used interchangeably to refer to the ability of the polymer or its degradation products to be removed by biological events, such as by fluid transport away from the site of implantation or by cellular activity (e.g., phagocytosis). The term “bioabsorbable” will generally be used in the following description to encompass resorbable, absorbable, bioresorbable, and biodegradable.

A “biocompatible” material is a material that is compatible with living tissue or a living system by not being undesirably toxic or injurious for an intended medical application.

The term “coating,” as used herein and unless otherwise indicated, refers generally to material attached to a medical device. Preferably, the coating is a releasable therapeutic agent, such as a taxane therapeutic agent, adhered to at least one surface of an implantable medical device. A coating can include material covering any portion of a medical device, and can be configured as one or more coating layers. A coating can have a substantially constant or a varied thickness and composition. Coatings can be adhered to any portion of a medical device surface, including the luminal surface, the abluminal surface, or any portions or combinations thereof.

The term “coating layer,” as used herein, refers to a stratified portion of a coating having a measurable composition distinguishable physically or chemically from an adjacent layer or material. Coating layers may be identified by one or more measurable properties (such as rate of elution, appearance, durability, infrared spectrum, crystal structure), and may be differentiated from an adjacent coating layer by at least one measurable property (e.g. different elution rates, chemical compositions, melting points, and the like). Coating layers are preferably substantially parallel and may be oriented parallel to a medical device surface. A coating layer material can be positioned in contact with the medical device surface, or in contact with other material(s) between the medical device surface and the coating layer material. A coating layer can cover any portion of the surface of a medical device, including material positioned in separate discrete portions of the medical device or as a continuous layer over an entire surface. Coatings and coating layers may also be at least partially confined within portions of a medical device, such as pores, holes of wells.

The phrase “Controlled release” refers to an alteration of the rate of release of a therapeutic agent from a medical device coating in a given environment. A coating or configuration that alters the rate at which the therapeutic agent is released from a medical device provides for the controlled release of the therapeutic agent. A “sustained release” refers to prolonging the rate or duration of release of a therapeutic agent from a medical device. The rate of a controlled release of a therapeutic agent may be constant or vary with time. A controlled release may be described with respect to a drug elution profile, which shows the measured rate at which the therapeutic agent is removed from a drug-coated device in a given elution medium (e.g., a solvent) as a function of time. A controlled release elution profile may include, for example, an initial burst release associated with the introduction of the medical device into the physiological environment, followed by a more gradual subsequent release. A controlled release can also be a gradient release in which the concentration of the therapeutic agent released varies over time or a steady state release in which the therapeutic agent is released in equal amounts over a certain period of time (with or without an initial burst release).

The term “effective amount” refers to an amount of an active ingredient sufficient to achieve a desired affect without causing an undesirable side effect. In some cases, it may be necessary to achieve a balance between obtaining a desired effect and limiting the severity of an undesired effect. It will be appreciated that the amount of active ingredient used will vary depending upon the type of active ingredient and the intended use of the composition of the present invention.

The term “elution,” as used herein, refers to removal of a material from a coating by contact with an elution medium. The elution medium can remove the material from the coating by any process, including by acting as a solvent with respect to the removable material. For example, in coated medical devices adapted for introduction to the vascular system, blood can act as an elution medium that dissolves a therapeutic agent releasably associated with a portion of the surface of the medical device. The therapeutic agent can be selected to have a desired solubility in a particular elution medium. Unless otherwise indicated, the term “release” referring to the removal of the therapeutic agent from a coating in contact with an elution medium is intended to be synonymous with the term “elution” as defined above. Similarly, an “elution profile” is intended to be synonymous with a “release profile,” unless otherwise indicated.

An “elution medium,” as used herein, refers to a material (e.g., a fluid) that removes a therapeutic agent from a coating upon contact of the coating with the elution medium for a desired period of time. A suitable elution medium is any substance or change in conditions (e.g., increased temperature, changing pH, and the like) causing the therapeutic agent to be released from the coating. The elution medium is desirably a fluid. More desirably, the elution medium is a biological fluid such as blood or porcine serum, although any other chemical substance can be used as an elution medium. For example, alternative elution media include phosphate buffered saline, an aqueous solution including a cyclodextrin such as Heptakis-(2,6-di-O-methyl)-β-cyclodextrin (HCD), Sodium Dodecyl Sulfate (SDS) and reaction conditions including elevated temperature and/or changes in pH, or combinations thereof, that release the therapeutic agent at a desired rate. Preferably, the elution medium is a fluid that provides an elution profile that is similar to the elution profile obtained upon implantation of the medical device within a body vessel and/or a desired time period for elution. For example, porcine serum can provide an elution profile that is similar to the elution profile in blood for some coating configurations.

A therapeutic agent is “enclosed” if the therapeutic agent is surrounded by the coating or other portions of the medical device, and does not form a portion of the surface area of the medical device prior to release of the therapeutic agent. When a medical device is initially placed in an elution medium, an enclosed therapeutic agent is preferably not initially in contact with the elution medium.

The term “hydrophobic,” as used herein, refers to a substance with a solubility in water of less than 0.1 mg/mL at room temperature (about 25° C.).

The term “luminal surface,” as used herein, refers to the portion of the surface area of a medical device defining at least a portion of an interior lumen. Conversely, the term “abluminal surface,” as used herein, refers to portions of the surface area of a medical device that do not define at least a portion of an interior lumen. For example, where the medical device may be a vascular stent having a cylindrical frame formed from a plurality of interconnected struts and bends defining a cylindrical lumen, the abluminal surface can include the exterior surface, sides and edges of the struts and bends, while the luminal surface can include the interior surface of the struts and bends.

The term “interface,” as used herein, refers to a common boundary between two structural elements, such as two coating layers in contact with each other.

The term “implantable” refers to an ability of a medical device to be positioned at a location within a body, such as within a body vessel. Furthermore, the terms “implantation” and “implanted” refer to the positioning of a medical device at a location within a body, such as within a body vessel.

The term “mixture” refers to a combination of two or more substances in which each substance retains its own chemical identity and properties.

A “non-bioabsorbable” or “biostable” material refers to a material, such as a polymer or copolymer, which remains in the body without substantial bioabsorption.

The term “pharmaceutically acceptable,” as used herein, refers to those compounds of the present invention which are, within the scope of sound medical judgment, suitable for use in contact with the tissues of humans and lower mammals without undue toxicity, irritation, and allergic response, are commensurate with a reasonable benefit/risk ratio, and are effective for their intended use, as well as the zwitterionic forms, where possible, of the compounds of the invention.

As used herein, the term “solid form” in reference to taxane molecules refers to an arrangement of molecules comprising a core taxane structure in the solid phase, including any polymorph or solvate crystal solid structure. Solid forms can include solvated crystalline forms comprising water molecules positioned between taxane molecules, non-crystalline amorphous taxane molecular arrangements or anhydrous taxane molecular arrangements substantially free of water molecules. Examples of solid forms of paclitaxel taxane molecules include anhydrous paclitaxel, amorphous paclitaxel and dihydrate paclitaxel.

As used herein, the phrase “therapeutic agent” refers to any implantable pharmaceutically active agent that results in an intended to provide a therapeutic effect on the body to treat or prevent conditions or diseases.

When naming substances that can exist in multiple enantiomeric forms, reference to the name of the substance without an enantiomeric designation, such as (d) or (l), refers herein to the genus of substances including the (d) form, the (l) form and the racemic mixture (e.g., d,l), unless otherwise specified. For example, recitation of “poly(lactic acid),” unless otherwise indicated, refers to a compound selected from the group consisting of: poly(L-lactic acid), poly(D-lactic acid) and poly(D,L-lactic acid). Similarly, generic reference to compounds that can exist in two or more polymorphs is understood to refer to the genus consisting of each individual polymorph species and any combinations or mixtures thereof.

As used herein, “derivative” refers to a chemically or biologically modified version of a chemical compound that is structurally similar to a parent compound and (actually or theoretically) derivable from that parent compound. A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, as well as salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analog” or “analogue” refer to a chemical compound that is structurally similar to another but differs slightly in composition (as in the replacement of one atom by an atom of a different element or in the presence of a particular functional group), but may or may not be derivable from the parent compound. A “derivative” differs from an “analog” in that a parent compound may be the starting material to generate a “derivative,” whereas the parent compound may not necessarily be used as the starting material to generate an “analogue.”

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to one polymer or a mixture comprising two or more polymers.

### Methods of Detecting Taxane Therapeutic Agents

The present disclosure provides methods for measuring the release of a taxane therapeutic agent from a medical device coating in an elution medium as a function of time to obtain an elution profile. The elution profile may be indicative of the configuration of the coating (e.g., solid forms or number of coating layers). The elution medium is preferably formulated to provide an elution profile indicative of the structure or composition of the medical device coating. The elution profile may provide a measure of the amount of the taxane therapeutic agent released from the medical device coating as a function of time the coating is in contact with the elution medium.

The elution profile obtained from contacting the coating with a cyclodextrin elution medium are useful, for example, in obtaining information about the coating for lot release testing. The elution profile in a cyclodextrin elution medium may be used to detect the configuration, composition or amount of the taxane therapeutic agent present on a coated medical device, or for measuring the elution rate and elution kinetics of the taxane therapeutic agent from the medical device. For example, certain β-cyclodextrin compounds elute paclitaxel medical device coatings at a desirable rate and with a predictability suitable for use in lot release testing for the purpose of differentiating between amorphous or solvated dihydrate solid forms of paclitaxel in the coating, or measuring total paclitaxel dose in the coating. Further, elution media comprising β-cyclodextrin compounds are suitable for providing distinguishable paclitaxel elution profiles from medical device coatings comprising a combination of paclitaxel with different amounts of a release modifying agent, including a biodegradable elastomer such as poly(lactic acid).

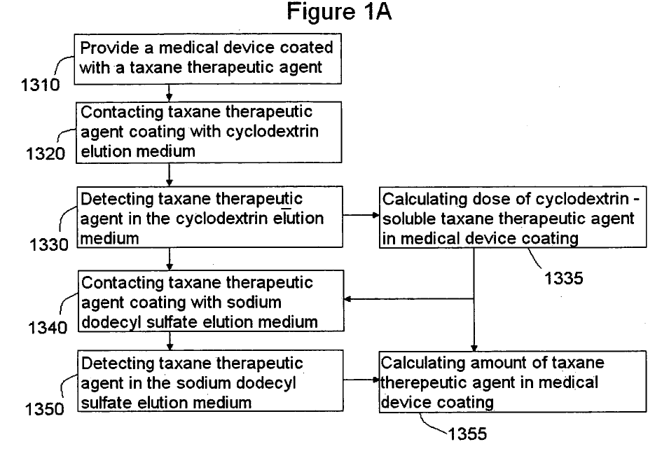

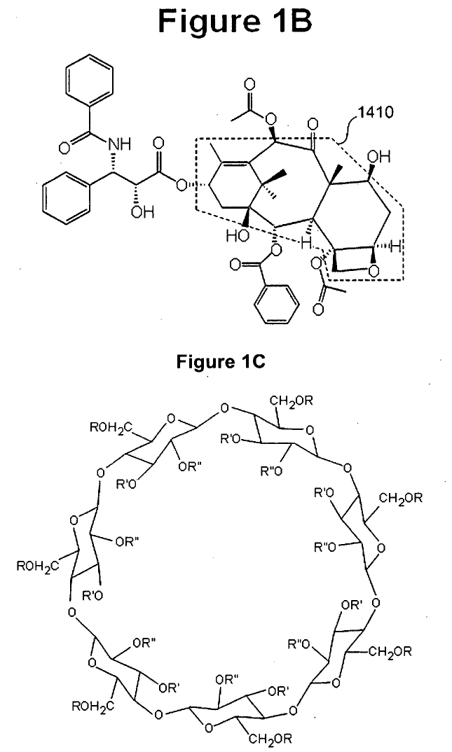

FIG. 1A is a schematic flow diagram of certain preferred (destructive testing) methods for detecting the elution profile of a taxane therapeutic agent from a medical device coating. Preferably, the methods comprise the step 1310 of providing a medical device coated with a taxane therapeutic agent, step 1320 of contacting the taxane therapeutic agent coating with a cyclodextrin elution medium and step 1330 of detecting the taxane therapeutic agent. The steps may be performed in any suitable order, and may include one or more intervening steps.

The coated medical device provided in step 1310 comprises a taxane therapeutic agent that is released in an elution medium containing a cyclodextrin. The coated medical device that is provided in step 1310 is preferably a representative sample of a group of coated stents (i.e., a sample for lot testing). Optionally, the method may further comprise the step(s) of coating a medical device with a taxane therapeutic agent, for example to prepare a standard for comparative testing of samples with unknown coating composition. The taxane therapeutic agent can be coated on, or incorporated into any portion of the medical device in any suitable manner or configuration. Preferably, the taxane therapeutic agent is present in one or more coating layers coated on at least one surface of the medical device, although the taxane therapeutic agent can also be contained within the medical device itself. The medical device can have any suitable configuration, but is preferably configured for implantation within a body vessel from a delivery catheter, such as a stent, stent graft or valve. The taxane therapeutic agent can be applied with or without other materials, such as biodegradable or biostable polymers. For obtaining lot release data, the coated medical device provided in step 1310 can be a representative example of a multiple coated medical devices prepared in the same manner. The representative coated medical device coating is typically removed during the process of contacting the coating with one or more suitable elution media.

The coated medical device is contacted with an elution medium comprising a cyclodextrin in step 1320 under elution conditions such as temperature, pressure and fluid flow rate that permit elution of the taxane therapeutic agent from the coated medical device at a desired rate. The elution medium is preferably a liquid solution comprising a cyclodextrin in a concentration adequate to elute the taxane therapeutic agent over a desired time period. In addition, the elution conditions and elution medium composition are preferably selected to provide a taxane therapeutic agent elution profile that differs depending on the structure or composition of the coating. For example, as discussed below, certain cyclodextrin elution media provide a more rapid elution of amorphous paclitaxel than dihydrate paclitaxel. The elution medium may be contacted with the coating in any suitable manner, including placement of the coated medical device in a reservoir of the elution medium or flowing the elution medium past the coated medical device.

The taxane therapeutic agent may be detected in the elution medium according to step 1330 by any suitable method that identifies the presence of the taxane therapeutic agent, including ultraviolet (UV) detection or HPLC detection. Preferred methods permit detection of the taxane therapeutic agent as a function of time the coating is in contact with the elution medium. For example, an elution medium may continuously flow past a coated medical device, and be collected as samples of equal volume at regular intervals after contact with the coating. The concentration of the taxane therapeutic agent in each sample can be measured by detecting the optical density of each sample using ultraviolet spectrophotometry to measure the absorbance of the sample at a peak characteristic of the taxane therapeutic agent.

Referring again to FIG. 1A, the total amount of taxane therapeutic agent soluble in the cyclodextrin elution medium may be calculated in step 1335 by any suitable method, based on the detection method used to detect the taxane therapeutic agent in step 1330. For example, the optical density of the elution medium measured by UV detection after contact with the taxane therapeutic agent can be converted to the total amount of the taxane therapeutic agent released from the coating. Preferably, the amount of taxane therapeutic agent soluble in the cyclodextrin solution may be correlated to the structure of the coating. For example, as described below, the elution profile of a paclitaxel coating in a low-solubility dihydrate solid form in an aqueous HCD elution medium is readily distinguishable from the elution profile of a paclitaxel coating in the more readily soluble amorphous paclitaxel solid form. Similarly, paclitaxel coatings comprising various mixtures of the dihydrate paclitaxel solid form and the amorphous paclitaxel solid form can also be distinguished in an aqueous HCD elution medium. Accordingly, the amount of the amorphous paclitaxel solid form in a paclitaxel coating can be estimated from the elution profile in aqueous HCD elution media.

The coated medical device may also be contacted with elution media that contain substances that dissolve a taxane therapeutic agent more or less readily than cyclodextrin. For example, elution media can include substances that rapidly dissolve a taxane therapeutic agent, such as sodium dodecyl sulfate (SDS), or ethanol with or without a cyclodextrin. Step 1340 provides for contacting the coating comprising a taxane therapeutic agent with SDS, and is preferably performed after contacting the coating with a cyclodextrin elution medium without SDS. Preferably, taxane therapeutic agent in the coating that is not sufficiently soluble during contact with the cyclodextrin elution medium in step 1320 for detection in step 1330 is rapidly dissolved upon contact with the SDS elution medium in step 1340, and subsequently detected in step 1350. Detection of the taxane therapeutic agent present in the SDS elution medium in step 1350 is performed by any suitable technique, which includes the methods used in step 1330. Similarly, the amount of the taxane therapeutic agent detected in step 1355 can be correlated to the amount of taxane therapeutic agent remaining in the coating after contact with the cyclodextrin elution medium in step 1320 in a manner described for step 1335 above.

In one embodiment, the elution profile of a paclitaxel coating on a medical device is determined by first contacting the medical device with a cyclodextrin elution medium comprising Heptakis-(2,6-di-O-methyl)-β-cyclodextrin (HCD) that readily dissolves the amorphous paclitaxel, and subsequently detecting the amount of taxane therapeutic agent within the elution medium. Preferably, the amorphous paclitaxel solid form dissolves about 10-times more rapidly in the HCD cyclodextrin elution medium than the dihydrate paclitaxel solid form. The medical device is exposed to the cyclodextrin elution medium and the rate of release of the taxane therapeutic agent from the medical device is determined by detecting the taxane therapeutic agent in the cyclodextrin elution medium for a first desired period of time, which is preferably about 2 hours or less. After the first desired period of time, the amount of taxane therapeutic agent remaining on the medical device can be determined by contacting the medical device with an SDS elution medium that readily dissolves the remaining paclitaxel, including paclitaxel in the dihydrate solid form, and subsequently detecting the amount of taxane therapeutic agent dissolved in the SDS elution medium.

### Taxane Therapeutic Agents

The taxane therapeutic agent can have various molecular structures, but is preferably paclitaxel or a paclitaxel derivative. While preferred embodiments are described herein with relation to paclitaxel, these embodiments are also applicable to any taxane therapeutic agent. FIG. 1B and structure (1) below show the molecular structure of paclitaxel, which comprises a core ring structure of four fused rings, enclosed by box 1410 and shaded in structure (1) below. Taxanes in general, and paclitaxel in particular, are taxane therapeutic compounds considered to function as a cell cycle inhibitors by acting as an anti-microtubule agent, and more specifically as a stabilizer. As used herein, the term “paclitaxel” refers to a compound of the chemical structure shown as structure (1) below, consisting of a core structure with four fused rings (“core taxane structure,” shaded in structure (1)), with several substituents.

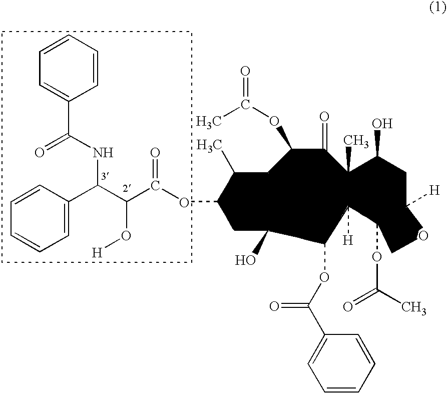

Other taxane analog or derivative compounds are characterized by variation of the paclitaxel structure (1). Preferred taxane analogs and derivatives core vary the substituents attached to the core taxane structure. In one embodiment, the therapeutic agent is a taxane analog or derivative including the core taxane structure (1) and the methyl 3-(benzamido)-2-hydroxy-3-phenylpropanoate moiety (shown in structure (2) below) at the 13-carbon position (“C13”) of the core taxane structure (outlined with a dashed line in structure (1)).

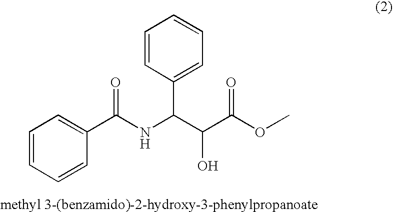

It is believed that structure (2) at the 13-carbon position of the core taxane structure plays a role in the biological activity of the molecule as a cell cycle inhibitor. Examples of therapeutic agents having structure (2) include paclitaxel (Merck Index entry 7117), docetaxol (TAXOTERE, Merck Index entry 3458), and 3′-desphenyl-3′-(4-nitrophenyl)-N-debenzoyl-N-(t-butoxycarbonyl)-10-deacetyltaxol.

A composition comprising a taxane compound can include formulations, prodrugs, analogues and derivatives of paclitaxel such as, for example, TAXOL (Bristol Myers Squibb, New York, N.Y.), docetaxel, 10-desacetyl analogues of paclitaxel and 3′-N-desbenzoyl-3′-N-t-butoxy carbonyl analogues of paclitaxel. Paclitaxel has a molecular weight of about 853 amu, and may be readily prepared utilizing techniques known to those skilled in the art (see, e.g., Schiff et al., Nature 277: 665-667, 1979; Long and Fairchild, Cancer Research 54: 4355-4361, 1994; Ringel and Horwitz, J. Nat'l Cancer Inst. 83 (4): 288-291, 1991; Pazdur et al., Cancer Treat. Rev. 19 (4): 351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35 (52): 9709-9712, 1994; J. Med. Chem. 35: 4230-4237, 1992; J. Med. Chem. 34: 992-998, 1991; and J. Natural Prod. 57 (10): 1404-1410, 1994; J. Natural Prod. 57 (11): 1580-1583, 1994; J. Am. Chem. Soc. 110: 6558-6560, 1988), or obtained from a variety of commercial sources, including for example, Sigma Chemical Co., St. Louis, Mo. (T7402—from Taxus brevifolia).

Preferably, the taxane solid forms are selected from the group consisting of: amorphous taxane therapeutic agent, anhydrous taxane therapeutic agent and dihydrate therapeutic agent. The taxane therapeutic agent is preferably paclitaxel. Solid forms of taxane therapeutic agents in medical device coatings can have identical molecular structures, but differ in the arrangement of the taxane molecules in the coating. Bulk samples of three different solid forms of the taxane therapeutic agent (amorphous, anhydrous or dihydrate) can be formed by dissolving the solid taxane therapeutic agent, typically obtained in the amorphous form, in different solvents, as described below. Different solid forms of paclitaxel can also be prepared and identified by the methods described in J. H. Lee et al, “Preparation and Characterization of Solvent Induced Dihydrated, Anhydrous and Amorphous Paclitaxel,” Bull. Korean Chem. Soc., v. 22, no. 8, pp. 925-928 (2001), which is incorporated herein by reference.

The solid forms of the taxane therapeutic agent can also be identified and differentiated on the basis of one or more physical properties including melting point, solubility and appearance. Suitable solvent systems for the synthesis of amorphous, dihydrate and anhydrous taxane therapeutic solid forms, as well as characteristic melting point ranges and infrared spectrum peaks useful in identifying each solid form, are provided in Table 1.

TABLE 1
Preparation and Identification of Taxane Solid Forms

| | Desired Taxane Solid Form | | |
|---|---|---|---|
| | Amorphous | Anhydrous | Dihydrate |
| Solvent: | Dichloromethane | Methanol/ | Methanol/Water |
| | | Hexane | |
| Melting Point: | 190-210° C. | 220-221° C. | 209-215° C. |
| Characteristic | Single peak | Two peaks | Three or more |
| IR peaks: | between 1700- | between 1700- | peaks between |
| | 1740 cm⁻¹ | 1740 cm⁻¹ | 1700-1740 cm⁻¹ |
| | 3064 cm⁻¹ (104), | 3065 cm⁻¹ (308), | 3067 cm⁻¹ (210), |
| | 3029 cm⁻¹ (106), | 2944 cm⁻¹ (310) | 3017 cm⁻¹ (212), |
| | 2942 cm⁻¹ (108) | | 2963 cm⁻¹ (214) |
| | 1650 cm⁻¹ (110) | 1646 cm⁻¹ (306) | 1639 cm⁻¹ (206) |
| | 1517 cm⁻¹ (112) | 1514 cm⁻¹ (312) | 1532 cm⁻¹ (208) |

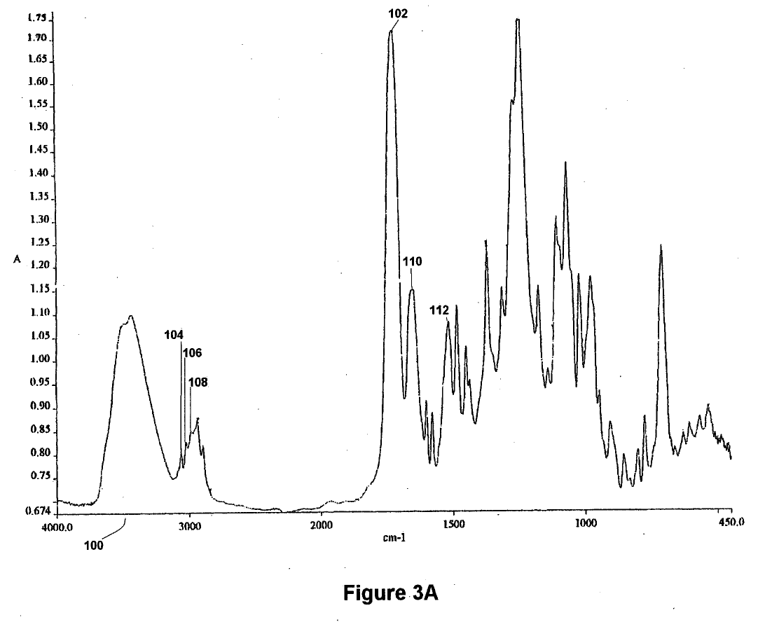

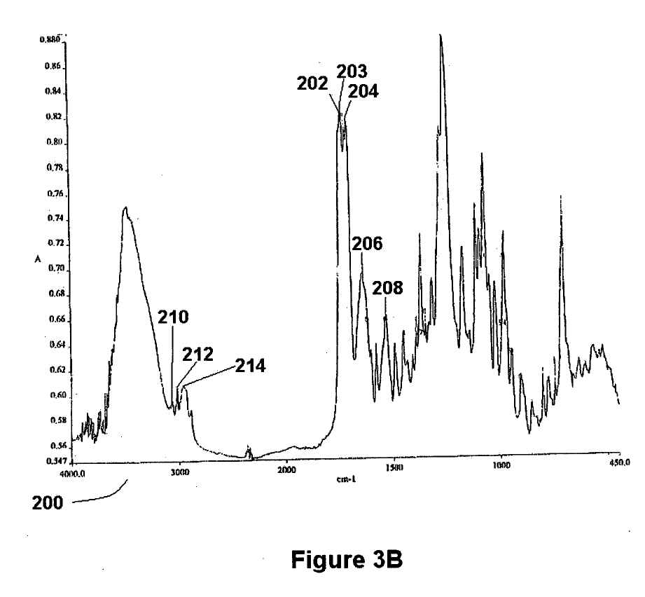

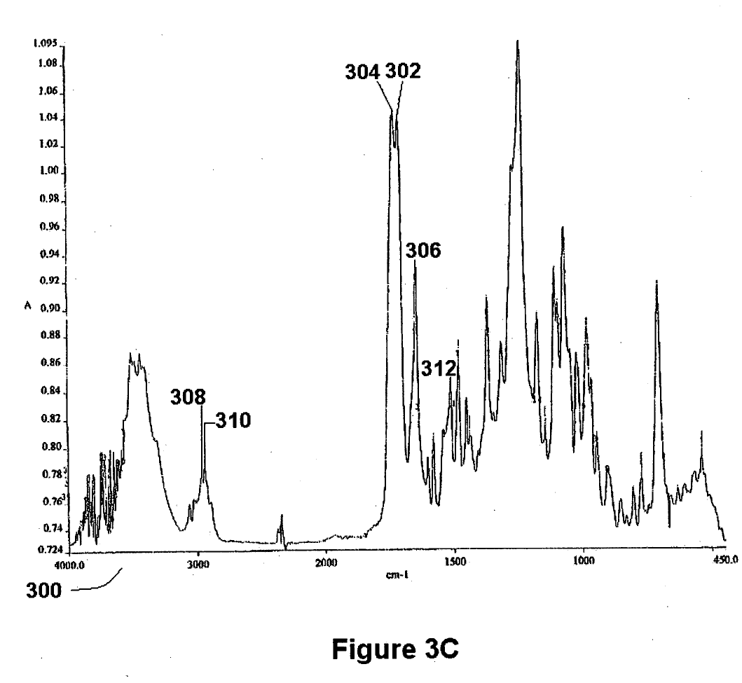

FIG. 3A shows an infrared vibrational spectrum of amorphous paclitaxel. The spectrum of amorphous paclitaxel 100 includes a single broad peak at about 1723 cm-1 (102), as well as the following other characteristic peaks: 3064 cm-1 (104), 3029 cm-1 (106), 2942 cm-1 (108), 1650 cm-1 (110), and 1517 cm-1 (112). The melting points of the amorphous paclitaxel samples prepared according to Example 1 were about 190° C.-210° C. An amorphous taxane therapeutic agent can be identified by the presence of a single broad peak between about 1700-1740 cm-1 in the infrared spectrum, typically at about 1723 cm-1. The amorphous taxane therapeutic agent was found to be more soluble in porcine serum than the dihydrate taxane therapeutic agent, but less soluble than the anhydrous taxane therapeutic agent.

FIG. 3B shows an infrared vibrational spectrum of dihydrate paclitaxel. The spectrum of dihydrate paclitaxel 200 includes three or more peaks between about 1700-1740 cm⁻¹, typically three peaks at about 1705 cm⁻¹ (204), about 1716 cm⁻¹ (203) and about 1731 cm⁻¹ (202), as well as the following other characteristic peaks: 3067 cm⁻¹ (210), 3017 cm⁻¹ (212), 2963 cm⁻¹ (214), 1639 cm⁻¹ (206), and 1532 cm⁻¹ (208). The melting points of the dihydrate paclitaxel samples prepared according to Example 1 were about 209° C.-215° C. Dehydration of dihydrate paclitaxel has been reported during heating at a rate of 10° C./min over a temperature range of between about 35° C. and about 100° C. measured by DSC (with peaks observed at about 50° C. and about 72° C.), and between about 25° C. and about 85° C. measured by Thermogravimetric Analysis (TGA), with lower temperatures reported at slower heating rates. R. T. Liggins et al., “Solid-State Characterization of Paclitaxel,” Journal of Pharmaceutical Sciences, v. 86, No. 12, pp. 1458-1463, 1461 (December 1997) (“Liggins”). The dihydrate paclitaxel has been reported to not show weight loss or evidence of dehydration when stored for several weeks when stored at 25° C. at 200 torr. Liggens et al., page 1461. The solubility of the bulk sample of dihydrate taxane therapeutic agent may be measured in various elution media to obtain a dihydrate control elution profile. The elution profile of a taxane therapeutic agent measure in the elution media may be compared to the dihydrate control elution profile to identify the amount of dihydrate solid form present in a taxane therapeutic agent coating to identify the amount of the dihydrate present in the coating by comparison with the dihydrate control elution profile.

FIG. 3C shows an infrared vibrational spectrum of anhydrous paclitaxel. The spectrum of anhydrous paclitaxel 300 includes a pair of peaks between about 1700-1740 cm⁻¹, typically two peaks at about 1714 cm⁻¹ (302) and about 1732 cm⁻¹ (304), as well as the following other characteristic peaks: 3065 cm⁻¹ (308), 2944 cm⁻¹ (310), 1646 cm⁻¹ (306), and 1514 cm⁻¹ (312). The melting points of the anhydrous paclitaxel samples prepared according to Example 1 were about 220° C.-221° C. The anhydrous taxane therapeutic agent was found to be more soluble in porcine serum than the amorphous taxane therapeutic agent, and significantly more soluble than the dihydrate taxane therapeutic agent.

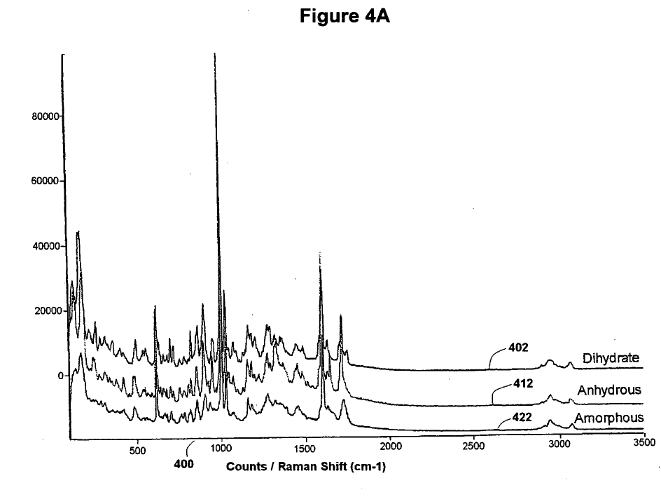

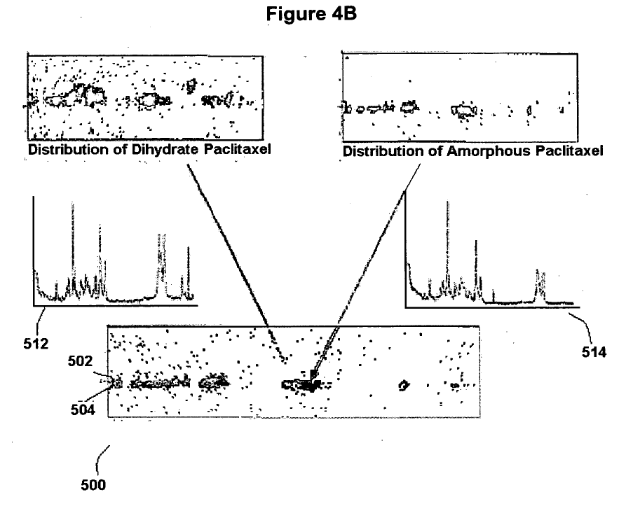

Differentiation of taxane solid states by vibrational spectroscopy can also be performed using Raman scattering. Raman scattering describes the phenomenon whereby incident light scattered by a molecule is shifted in wavelength from the incident wavelength. The magnitude of the wavelength shift depends on the vibrational motions the molecule is capable of undergoing, and this wavelength shift provides a sensitive measure of molecular structure. That portion of the scattered radiation having shorter wavelengths than the incident light is referred to as anti-Stokes scattering, and the scattered light having wavelengths longer than the incident beam as Stokes scattering. Raman scattering is a spectroscopic method useful for the detection of coatings, as the Raman spectra of different coatings or coating layers can be more distinct than the spectra obtained by direct light absorption or reflectance. FIG. 4A shows an overlay of three Raman spectral traces 400 recorded as an average of 10 spectra of three solid paclitaxel coatings on a stainless steel surface using a FT-Raman spectrometer, with excitation from a 532 nm laser with a power output of 8 mW. The three spectral traces correspond to the dihydrate (402), anhydrous (412) and amorphous (422) paclitaxel samples. Each spectral trace was collected over a 10 second integration each (total acquisition time of 100 seconds), using an air objective (100×, NA=0.9). Differences in the characteristic vibrational peaks can be used to differentiate the dihydrate, anhydrous and amorphous forms of the solid paclitaxel. The characteristic vibrational peaks correspond to the infrared characteristic peaks discussed with respect to the infrared spectra of FIGS. 3A-3C, and include the peaks listed in Table 1. Most notably, the presence of a single peak between 1700-1740 cm⁻¹ indicates the presence of an amorphous taxane therapeutic agent solid form, the presence of three or more peaks between 1700-1740 cm⁻¹ indicates the presence of the dihydrate taxane therapeutic agent solid form, and the presence of two peaks between 1700-1740 cm⁻¹ indicates the presence of the anhydrous taxane therapeutic agent solid form.

Confocal Raman microscopy allows improved axial and lateral resolution and fluorescence rejection over conventional Raman microscopy. Confocal Raman microscopy can be applied to reveal compositional or structural gradients as a function of depth within a sample. A depth profile of a coating can be obtained by confocal Raman microscopy by plotting the intensity of a component-specific vibrational band as a function of the distance from the sample surface. FIG. 4B shows a depth profile 500 of a coating comprising a mixture of dihydrate and amorphous solid forms of paclitaxel. The depth profile 500 was obtained by confocal Raman microscopy, by spatially detecting and plotting the intensity of scattered light matching a first spectrum 512 obtained from a dihydrate paclitaxel sample in a first color 502, followed by similarly detecting and plotting the intensity of scattered light matching a second spectrum 514 obtained from an amorphous paclitaxel sample. The depth profile 500 indicates that the dihydrate paclitaxel 502 is largely localized on the surface of the coating while the amorphous paclitaxel is predominantly distributed in a layer 504 below the dihydrate paclitaxel.

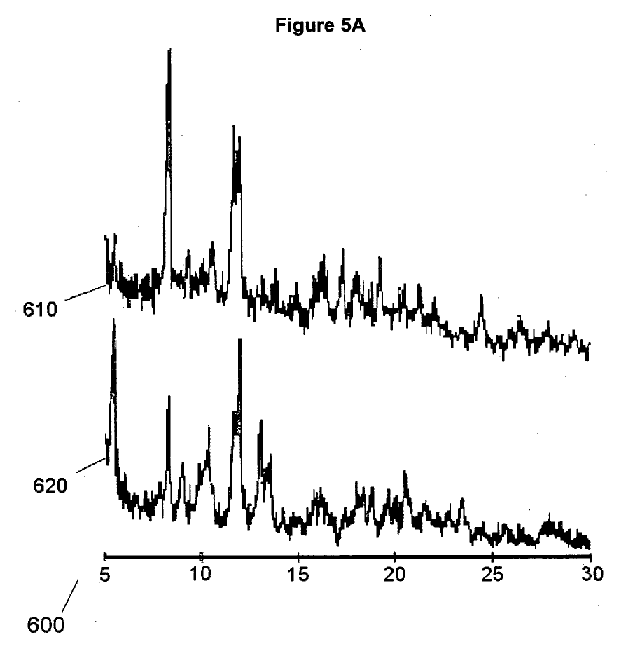

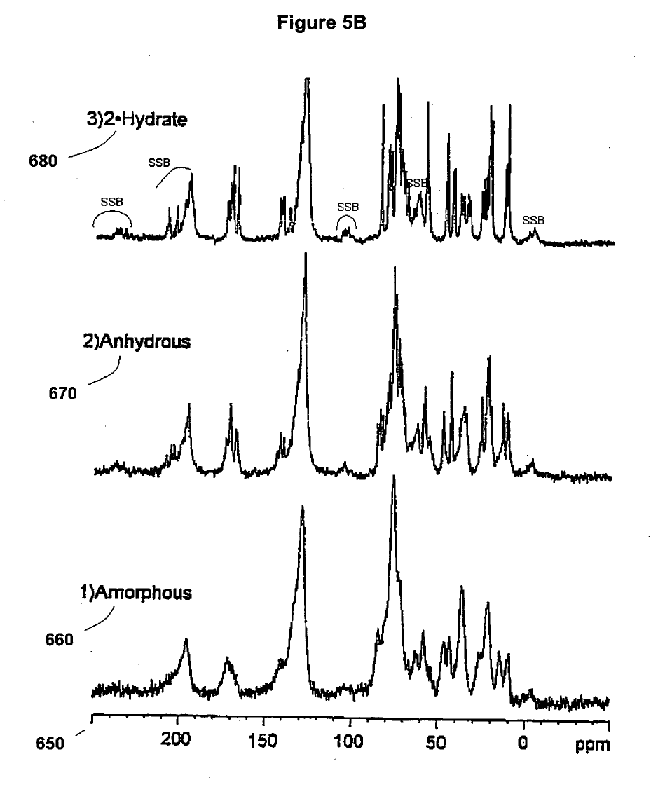

Powder X-ray Diffraction (XRPD) can also be used to differentiate various solid forms of taxane therapeutic agents. FIG. 5A shows the XRPD patterns 600 for amorphous 610 and dihydrate 620 solid forms of paclitaxel, with corresponding selected d-spacings of selected peaks provided in Table 2. Notably, the dihydrate paclitaxel can provide peaks different from the amorphous paclitaxel at 6.1, 9.5, 13.2 and 13.8° 2θ (obtained at 25° C.).

TABLE 2
XRPD Selected d-Spacings and Peak Intensities

| | d-spacing | | |
|---|---|---|---|
| °2θ | (Å) | Anhydrous | Dihydrate |
| 6.1 | 14.5 | | Strong* |
| 8.8 | 10.0 | Strong* | Strong* |
| 9.5 | 9.3 | | Medium** |
| 10.9 | 8.11 | | Medium** |
| 11.1 | 7.96 | Medium** | |
| 12.1 | 7.31 | Medium** | Strong* |
| 12.3 | 7.19 | Medium** | Strong* |
| 13.3 | 6.65 | | Medium** |
| 13.8 | 6.41 | | Medium** |
| 14.1 | 6.27 | Weak*** | |
| 19.3 | 4.59 | Weak*** | |
| 25.9 | 3.44 | Medium** | |

*= Strong Peak (relative intensity is more than 50);
**= Medium Peak (relative intensity between 20 and 50);
***= Weak Peak (relative intensity less than 20)

The data in FIG. 5A and Table 2 was obtained from R. T. Liggins et al., “Solid-State Characterization of Paclitaxel,” Journal of Pharmaceutical Sciences, v. 86, No. 12, pp. 1458-1463 (December 1997), which is incorporated herein by reference. As described by Liggins et al., the anhydrous sample 610 can be obtained by drying paclitaxel (Hauser, Boulder, Colo.) at ambient temperature and reduced pressure (200 torr) in a vacuum oven (Precision Scientific, Chicago, Ill.). Liggins et al report that the anhydrous sample 610 contained about 0.53% water, measured by Karl-Fischer analysis. The dihydrate sample 620 can be obtained by adding the anhydrous sample above to distilled water and stirring at ambient temperature for 24 hours, followed by filtration and collection of suspended solid paclitaxel and subsequent drying to constant weight. Liggins et al. report that the dihydrate sample 620 contained about 4.47% water (about 2.22 mol water/mol paclitaxel). Additional details relating to the spectra of FIG. 5A or the data in Table 2 are found in the Liggins et al. reference.

A ¹³C Nuclear Magnetic Resonance (NMR) can also be used to differentiate various solid forms of taxane therapeutic agents. FIG. 5B shows the ¹³C NMR spectra 650 for amorphous 660, anhydrous 670 and dehydrate 680 solid forms of paclitaxel. The data in FIG. 5B was obtained from Jeong Hoon Lee et al., “Preparation and Characterization of Solvent Induced Dihydrated, Anhydrous and Amorphous Paclitaxel,” Bull. Korean Chem. Soc. v. 22, no. 8, pp. 925-928 (2001), incorporated herein by reference. As described by Lee et al., the spectra 650 in FIG. 5B can be obtained using a cross polarization/magic angle spinning (CP/MAS) ¹³C solid form NMR (Bruker DSX-300, Germany) experiment operating at 75.6 MHz. Standard pulse sequences and phase programs supplied by Bruker with the NMR spectrometer can be used to obtain the spectra 650. For each sample, about 250 mg sample can be spun at about 5 kHz in a 4 mm rotor, and cross polarization can be achieved with contact time of 1 ms. This process can be followed by data acquisition over 35 ms with high proton decoupling. A three-second relaxation delay can be used. The spectra 650 are referenced to adamantane, using glycine as a secondary reference (carbonyl signal of glycine was 176.04 ppm). Referring to FIG. 5B, the ¹³C solid form NMR spectrum of the dihydrate paclitaxel 680 shows greater sharpness and peak splitting than either of the other solid forms of paclitaxel, the spectrum of the anhydrous paclitaxel 670 shows greater sharpness and peak splitting than the spectrum from amorphous paclitaxel 660, and the spectrum from amorphous paclitaxel 660 shows less resolution and peak splitting than the spectrum from anhydrous paclitaxel 670.

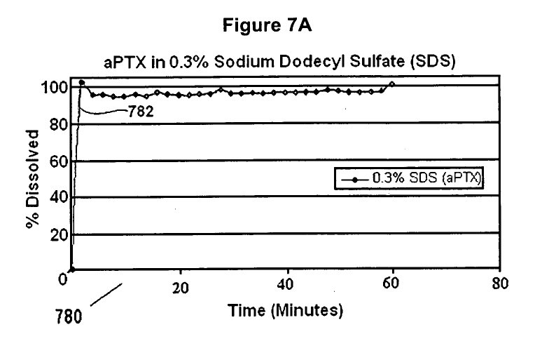

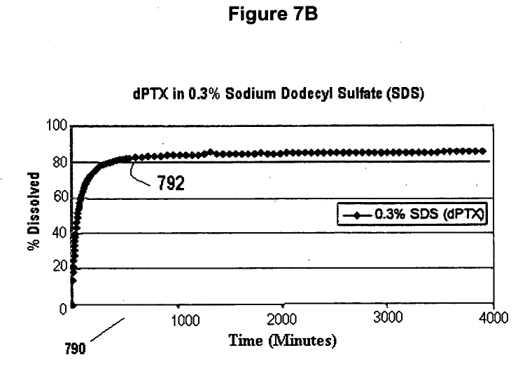

Solid forms of a taxane therapeutic agent may be identified by visual inspection of a coating. FIGS. 10A-13B are optical micrographs of durable paclitaxel coatings on stents comprising various mixtures of dPTX and aPTX. The ratio of amorphous to dihydrate paclitaxel in each coating was subsequently determined by monitoring a characteristic paclitaxel UV absorption peak (e.g., 227 nm) in an elution media in contact with the paclitaxel coated stents. This determination was performed by sequentially dissolving the coating in two different elution media separately contacted with the coating. First, the paclitaxel coating was contacted with stream of a first elution medium (a 0.5-1.0% w/w aqueous HCD solution) in which the amorphous solid form of paclitaxel is substantially more soluble than the dihydrate solid form of paclitaxel. Second, after elution of the paclitaxel from the stents in the first elution medium, the remaining paclitaxel coating (presumed to be the more soluble dihydrate) was contacted with a stream of a second elution medium (ethanol or a 0.3% w/w aqueous Sodium Dodecyl Sulfate solution), in the absence of the first elution medium, effective to readily dissolve the dihydrate solid form paclitaxel in the coating. Based on the comparative solubility of the dPTX and aPTX solid forms in the first and second elution media (see, e.g., FIG. 7A and FIG. 7B), the concentration of paclitaxel in the elution media was measured by UV detection (at 227 nm) to determine the ratio of paclitaxel solid forms originally present in the taxane coatings on the stents.

A mixture of amorphous and dihydrate taxane therapeutic agent coating has a cloudy or spotted appearance (clear coating with white opaque regions). FIG. 10A shows a 50× optical micrograph of a metal stent coated with about 65% dihydrate paclitaxel (35% amorphous paclitaxel) coating prepared by ultrasonic spray coating a 4.68 mM paclitaxel solution in a 93% v methanol (7% water) solvent. FIG. 10B shows a 115× optical micrograph of the coating shown in FIG. 10A. The 65:35 dPTX:aPTX coating has a largely cloudy and spotty appearance due to the presence of the dihydrate solid form of paclitaxel. Opaque white regions appear in the coating due to the mixture of the dihydrate (opaque, white) with lesser amounts of the amorphous (clear) solid form of paclitaxel.

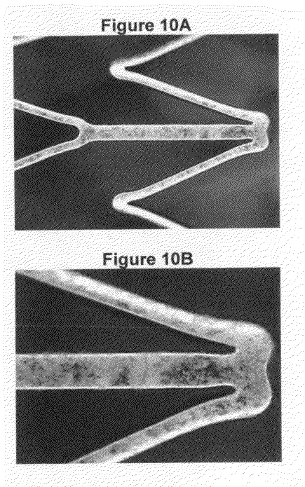

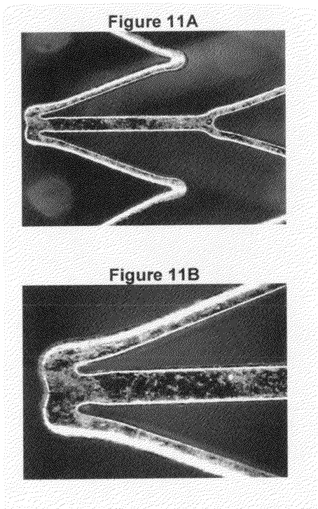

FIG. 11A shows a 50× optical micrograph of a metal stent coated with about 48% dihydrate paclitaxel and about 52% amorphous paclitaxel coating prepared by ultrasonic spray coating a 4.71 mM paclitaxel solution in a 93% w/w methanol (7% w/w water) solvent. FIG. 11B shows a 115× optical micrograph of the coating shown in FIG. 11A. The 48:52 w/w dPTX:aPTX coating has a total dose of paclitaxel of about 3 micrograms per mm2, as well as a clearer and less spotty appearance compared to the coating in FIGS. 10A-10B due to a more uniform distribution of the amorphous solid form of paclitaxel. Regions of varying opacity in the coating result from the non-uniform mixture of the amorphous solid form of paclitaxel with the dihydrate (opaque) solid form.

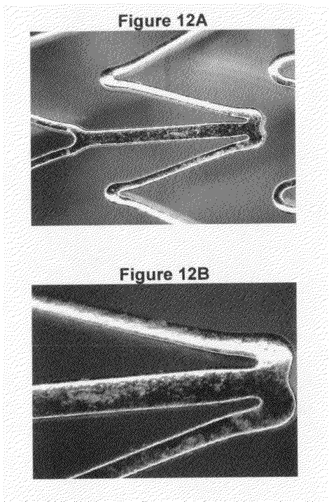

FIG. 12A shows a 50× optical micrograph of a metal stent coated with about 40% dihydrate paclitaxel (60% amorphous paclitaxel) coating prepared by ultrasonic spray coating a 4.68 mM paclitaxel solution in a 95% v methanol (5% water) solvent. FIG. 12B shows a 115× optical micrograph of the coating shown in FIG. 12A. The 40:60 w/w dPTX:aPTX coating has a clearer and less spotty appearance than the coating in FIGS. 10A-10B due to the increased proportion of the amorphous solid form of paclitaxel. Regions of varying opacity in the coating result from the mixture of the amorphous (clear) solid form of paclitaxel with the dihydrate (opaque, white) solid form.

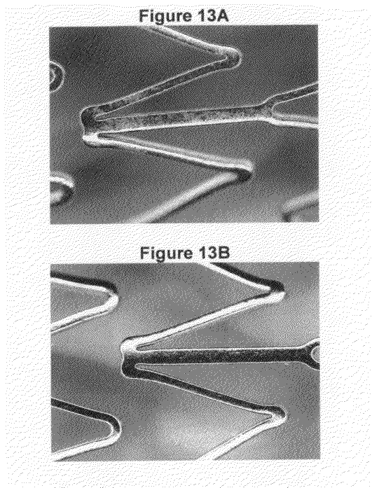

FIG. 13A shows a 50× optical micrograph of a metal stent coated with about 100% amorphous paclitaxel coating prepared by ultrasonic spray coating a 4.68 mM paclitaxel solution in a 95% v methanol (5% water) solvent. FIG. 13B shows a 115× optical micrograph of the coating shown in FIG. 13A. The aPTX coating has a clearer appearance indicative of the amorphous (clear) solid form of paclitaxel.

### Detecting Coating Configurations

A first embodiment provides methods of detecting the elution of a taxane therapeutic agent from a medical device coating comprising the taxane therapeutic agent in a desired solid form, such as a solid form of paclitaxel. Different solid forms of a substance may have the same molecular chemical structure, but different arrangements of molecules in the solid (such as different crystal structures). Taxane therapeutic agents can form at least three different solid forms, including an amorphous, anhydrous and solvated forms. The solvated form includes water molecules within the solid structure, such as the dihydrate paclitaxel solid form. Different solid forms of taxane therapeutic agents may have different solubility properties. Medical device coatings of taxane therapeutic agents can have different elution profiles depending on the solid form(s) present in the coating. Therefore, taxane therapeutic agents can be released in the body at different rates, depending on the solid form(s) of the taxane therapeutic agent present in the coating.

The different solid forms of the taxane therapeutic agent preferably contain one or more types of taxane therapeutic agent(s) arranged in different crystalline or non-crystalline forms in the coating, although a mixture of two or more taxane therapeutic agents can also be used. Preferably, the taxane therapeutic agent is paclitaxel. The solvated solid forms may further comprise water molecules to form a solvated solid form, such as dihydrate paclitaxel (paclitaxel.2H2O). The molar ratio between the taxane therapeutic agent and the waters of hydration in a solvated solid form may include integer ratios as well as non-integer ratios, such as 2.2H2O per paclitaxel water molecules. For example, the solvated solid form may be characterized by a molar ratio of about 1.0 to 5.0 water molecules per molecule of taxane therapeutic agent, including ratios 1.2, 1.3, 1.4, 1.5, 1.6, 1.7, 1.8, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 and 5.0, and higher, water molecules of hydration per molecule of taxane therapeutic agent in the solvated solid form.

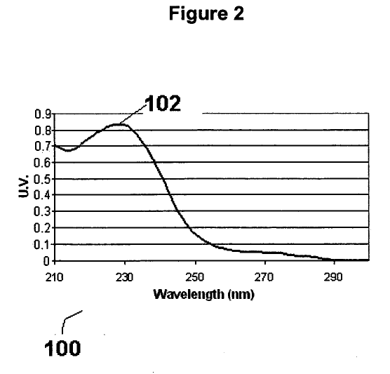

The presence of (and total amount of) a taxane therapeutic agent in any solid form in a coating can be identified by detecting the core taxane structure, for example by ultraviolet detection methods. For example, samples of the coating may be destructively tested by dissolving the coating in any suitable elution medium that permits measurement of a characteristic peak of the taxane therapeutic agent in solution in an ultraviolet (UV) spectrum of the taxane therapeutic agent in the solution. The characteristic peak is preferably associated with the core taxane structure. FIG. 2 shows an ultraviolet (UV) spectrum 100 (Agilent In-line UV Spectrophotometer) of paclitaxel in ethanol, obtained from a 25.67 micromolar solution of paclitaxel in ethanol. Taxane therapeutic agents such as paclitaxel provide a characteristic peak at 227 nm (102) indicative of the presence of the core taxane structure of paclitaxel in the solution. Taxane therapeutic agent can be identified from a UV spectrum of the elution medium characterized by the characteristic peak at about 227 nm, which can be correlated to the presence of the taxane therapeutic agent in the solution, regardless of the solid form from which the taxane molecule originated. Different solid forms of taxane therapeutic agents in medical device coatings can have identical molecular structures, but differ in the arrangement of the taxane molecules in the coating. Various solid forms of the taxane therapeutic agent can be identified and differentiated on the basis of one or more physical properties including melting point, solubility and appearance. In addition, various other analytical methods can be used to identify different solid forms of the taxane therapeutic agents, including vibrational spectroscopy (including Raman or Infrared Spectra), solubilities, melting points, X-ray Diffraction (XRD), 13C Nuclear Magnetic Resonance (NMR), and Temperature Programmed Desorption (TPD)).

Methods of detecting the release of taxane therapeutic agents from medical device coatings in a cyclodextrin elution medium are useful in performing lot release testing of medical devices to identify the solid form(s) of the taxane therapeutic agent present in a medical device coating by measuring the elution profile of the coated medical device in a suitable elution medium. A suitable elution medium can be any solvent system in which a desired medical device coating configuration has an elution profile that can be distinguished from the elution profile of a different, undesirable medical device coating configuration. A lot release criteria for evaluating a medical device coating may require that the elution profile of the taxane therapeutic agent from a medical device coating tested be sufficiently similar to the elution profile of a standard sample known to contain the desired solid form of the taxane therapeutic agent. Standard samples of the taxane therapeutic agent can be prepared and characterized in bulk form, and the elution profile of each solid form can be obtained in a suitable elution medium.

### Cyclodextrin Elution Media

The different solid forms of a taxane therapeutic agent may also be identified and differentiated from one another by differences in solubility in an elution medium. The elution medium preferably includes a cyclodextrin. A cyclodextrin is a cyclic oligosaccharide formed from covalently-linked glucopyranose rings defining an internal cavity. The diameter of the internal axial cavity of cyclodextrins increases with the number of glucopyranose units in the ring. The size of the glucopyranose ring can be selected to provide an axial cavity selected to match the molecular dimensions of a taxane therapeutic agent. Naturally occurring cyclodextrin molecules include α-, β- and γ-cyclodextrins having 6, 7 and 8 glucopyranose rings, respectively. The glucopyranose ring forms a cavity having a diameter of about 4.7-5.3 Angstroms for α-cyclodextrin, about 6.0 to 6.5 Angstroms for β-cyclodextrin, and about 7.5 to 8.3 Angstroms for γ-dextrins. See Sharma, U S et al., “Pharmaceutical and Physical Properties of Paclitaxel (Taxol) Complexes with Cyclodextrins,” Journal of Pharmaceutical Sciences, v. 84, no. 10, 1223-1230 (October 1995), incorporated by reference herein in its entirety. Without being bound by theory, it is believed that cyclodextrin molecules form complexes by enclosing a taxane therapeutic agent within the electron-rich, apolar interior axial cavity of the cyclodextrin molecule, while the hydrophilic perimeter of the cyclodextrin molecule is more readily solubilized through interaction with water molecules than the taxane therapeutic agent. Accordingly, the solubility of taxane therapeutic agents such as paclitaxel is typically increased in the presence of suitable cyclodextrin molecules.

The cyclodextrin is preferably a β-cyclodextrin. FIG. 1C shows a molecular formula of certain preferred β-cyclodextrin compounds. In the formula of FIG. 1C, R, R′ and R″ are independently selected from the group consisting of: —H, —OH, lower linear or branched alkyl (C₁-C₆) and alkoxy groups. Particularly preferred cyclodextrins for use with paclitaxel include β-cyclodextrin (R, R′ and R″ are all —H), Heptakis-(2,6-di-O-methyl)-β-cyclodextrin (HCD) (R, R′ are —CH₃ and R′ is —H), 2,3,6-trimethyl-b-cyclodextrin (R, R′ and R″ are all —CH₃), hydroxypropyl-β-cyclodextrin, and hydroxyethyl-β-cyclodextrin. While many embodiments described herein relate to the use of HCD in elution media in combination with paclitaxel, other cyclodextrin molecules and other taxane therapeutic agents may readily be substituted for HCD and/or paclitaxel within the scope of the invention. Suitable cyclodedtrin molecules also include other β-cyclodextrin molecules, as well as α- or γ-cyclodextrin structures. Elution media preferably comprise a cyclodextrin in a suitable liquid solvent. The solvent is preferably water, or a water-miscible liquid. The elution medium preferably comprises an amount of cyclodextrin effective to elute a taxane therapeutic agent at a desired rate. Preferred elution media comprise about 0.1% to about 10% of a cyclodextrin in water or a water-miscible liquid, including 0.1, 0.2, 0.3, 0.4, 0.5, 0.6, 0.7, 0.8, 0.9, 1.0, 2.0, 3.0, 4.0, 5.0, 6.0, 7.0, 8.0, 9.0 or 10.0% cyclodextrin, or higher, or intervals of 0.05% between 0.1% and 10%. Particularly preferred elution media comprise aqueous solutions of 0.1, 0.2, 0.3, 0.4, 0.5, 1.0, or 5.0% HCD. The elution media can be maintained at any suitable temperature, but is preferably between about 23° C. (room temperature) and 37° C. when contacted with a taxane therapeutic agent. Preferably, the elution media is maintained at about 25° C. when contacted with the taxane therapeutic agent.

### Obtaining Therapeutic Agent Elution Profiles

An elution profile is a graph of the percentage of a therapeutic agent released from a medical device coating as a function of time the coating is in contact with an elution medium. The rate of dissolution of the taxane therapeutic agent can vary based on the elution medium being used and the coating configuration. An elution profile can be obtained by any suitable method that allows for measurement of the release of the taxane therapeutic agent in a manner that can be measured with a desired level of accuracy and precision. In one embodiment, the elution profile of the release of a taxane therapeutic agent is obtained by contacting the medical device with a suitable elution medium.

The presence of different solid forms of the taxane therapeutic agent in a medical device coating can also be identified by contacting the coating with an elution medium that selectively dissolves one solid form and/or coating configuration more readily than another. After elution in an elution medium, such as porcine serum or blood, the presence (and amount) of the taxane therapeutic agent can be determined, for example by using ultraviolet (UV) spectroscopy or high pressure liquid chromatography (HPLC).

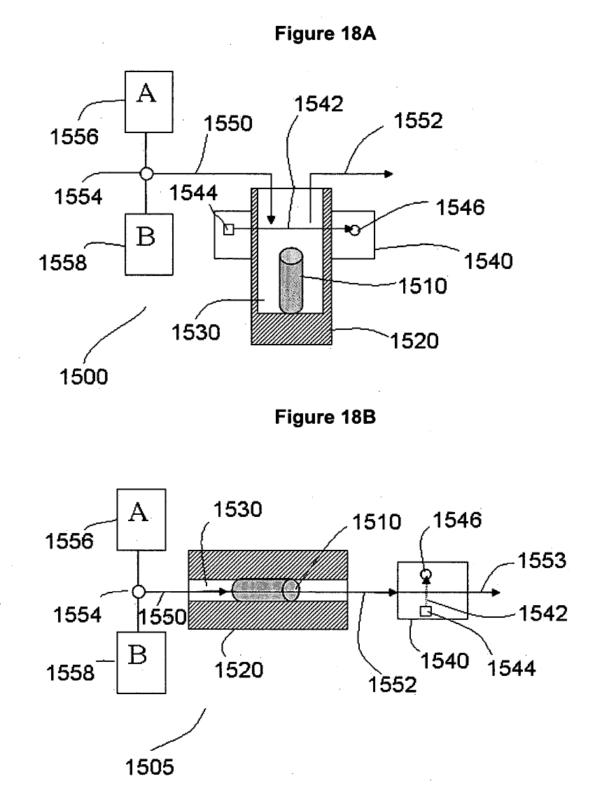

The release characteristics of a coated taxane therapeutic agent can be described by an elution profile. The elution profile of a medical device comprising a taxane therapeutic agent may indicate the percentage of the taxane therapeutic agent that dissolves as a function of time in a given elution medium. The rate of dissolution of the taxane therapeutic agent can vary based on the elution medium being used and the solid form of the taxane therapeutic agent before dissolution. An elution profile can be obtained by any suitable method that allows for measurement of the release of the taxane therapeutic agent from the coating in a manner that can be measured with a desired level of accuracy and precision. For example, FIG. 18A and FIG. 18B are schematics of two device configurations that can be used to obtain the elution profile of coated medical devices comprising taxane therapeutic agents.

In one embodiment, the elution profile of the release of a taxane therapeutic agent is obtained by contacting the medical device with a suitable elution medium. The elution medium can be formulated to simulate conditions present at a particular point of treatment within a body vessel. For example, an elution medium comprising porcine serum can be used to simulate implantation within a blood vessel. The release of taxane therapeutic agent from the medical device can be measured by any suitable spectrographic method, such as measurement of a UV absorption spectrum of the test fluid after contacting the medical device. Typically, the intensity of absorption at characteristic UV absorption peak, such as about 227 nm, can be correlated to the presence and amount of a taxane therapeutic agent in a sample. The amount of taxane therapeutic agent on the medical device can be determined by contacting the medical device with a suitable elution medium and detecting the amount of taxane therapeutic agent released from the medical device into the elution medium.

An elution medium can be selected to solubilize one solid form of a taxane therapeutic agent more rapidly than other solid forms of the taxane therapeutic agent, while allowing for subsequent measurement of the solubilized taxane therapeutic agent in a manner that can be correlated to the amount of the more soluble solid form of the taxane therapeutic agent released from the medical device. Subsequently, a second elution medium can be selected to quickly solubilize one or more other solid forms of the taxane therapeutic agent that did not dissolve in the first elution medium. Preferably, substantially all the taxane therapeutic agent of at least one solid form is removed from the medical device after contact with an elution medium for a desired period of time. The taxane therapeutic agent is subsequently detected in the elution medium. The detection of the taxane therapeutic agent is correlated to the amount of a particular solid form of the taxane therapeutic agent that was present on the medical device surface prior to contacting the medical device with the elution medium.

In one embodiment, the elution profile of a paclitaxel coating on a medical device is determined by first contacting the medical device with a first elution medium that readily dissolves the amorphous paclitaxel at least about 10-times more rapidly than the dihydrate paclitaxel, and then subsequently detecting the amount of taxane therapeutic agent within the elution medium. The medical device is exposed to the first elution medium and the rate of release of the taxane therapeutic agent from the medical device is determined by detecting the taxane therapeutic agent in the first elution medium for a first desired period of time. After the first desired period of time, the amount of taxane therapeutic agent remaining on the medical device can be determined by contacting the medical device with a second elution medium that readily dissolves the dihydrate paclitaxel, and subsequently detecting the amount of taxane therapeutic agent leaving the medical device in the second elution medium.

Any suitable analytical technique(s) may be used to detect a taxane therapeutic agent in an elution medium. Suitable detection methods, such as a spectrographic technique, permit measurement of a property of the elution medium that can be correlated to the presence or concentration of the taxane therapeutic agent with a desired level of accuracy and precision. In one embodiment, absorption spectroscopy (e.g., UV) can be used to detect the presence of a taxane therapeutic agent, such as in an elution medium. Accordingly, the Beer-Lambert Correlation may be used to determine the concentration of a taxane therapeutic agent in a solution. This correlation is readily apparent to one of ordinary skill in the art, and involves determining the linear relationship between absorbance and concentration of an absorbing species (the taxane therapeutic agent in the elution medium). Using a set of standard samples with known concentrations, the correlation can be used to measure the absorbance of the sample. A plot of concentration versus absorbance can then be used to determine the concentration of an unknown solution from its absorbance. UV absorbance of the taxane therapeutic agent at 227 nm can be measured (see FIG. 2), and the absorbance at this wave length can be correlated to concentration of the taxane in the test solution

FIG. 18A shows the first device configuration 1500 suitable for obtaining an elution profile in a batch processing manner. A coated medical device 1510 is placed in a fluid reservoir 1530 defined by a container 1520 having a fluid inlet line 1550 and a fluid outlet line 1552. The fluid reservoir 1530 can be a quartz vial. The fluid inlet line 1550 links the fluid reservoir 1530 to a first elution medium reservoir 1556 containing a first elution medium A, as well as a second elution medium reservoir 1558 containing a second elution medium B. A switch 1554 can be used to select the elution medium provided by the fluid inlet line 1550. The first elution medium can be selected to solubilize a taxane therapeutic agent in a first coating configuration more rapidly than other coating configurations, and to permit subsequent detection of the solubilized taxane therapeutic agent within the first elution medium. Preferably, the amount of the taxane therapeutic agent detected in the first elution medium can be correlated to the amount of the more soluble coating configuration that was present in the medical device coating. For example, when the medical device coating consists essentially of paclitaxel in one or more solid forms without a release modifying agent, the first elution medium preferably dissolves amorphous solid form of paclitaxel from a coated medical device 1510 more readily than dihydrate solid form of paclitaxel. Similarly, when the medical device coating comprises paclitaxel and a release modifying agent, such as a polymer, the first elution media preferably dissolves the coating at distinguishably rates depending on the amount of the release modifying agent present, or differences in the coating configuration. A preferred first elution medium is an aqueous solution comprising 0.1% to about 10% of a cyclodextrin, such as HCD.

The second elution medium B is preferably selected to dissolve the remaining taxane therapeutic agent that is not readily soluble in the first elution medium A. In one aspect, a single medical device coating can be contacted with the second elution medium B after being in contact with the first elution medium A, such that substantially all the taxane therapeutic agent is removed from the medical device after contact with the second elution medium B for a desired period of time. Alternatively, two different medical device coatings (or two separate portions of the same medical device coating) can be contacted with the second elution medium B only, without being contacted with the first elution medium A. In either case, the taxane therapeutic agent is subsequently detected within the second elution medium B, and the detection of the taxane therapeutic agent is correlated to the amount of a coating configuration of the taxane therapeutic agent that was present on the medical device coating prior to contacting the medical device with the second elution medium.

A detection means 1540 for detecting the taxane therapeutic agent can be used to detect the concentration of the taxane therapeutic agent in the elution medium in the fluid reservoir 1530. The detection means 1540 can be a UV detection apparatus comprising a UV light source 1544 and a UV light detector 1546 positioned and configured to provide a UV light path 1542 extending through the elution medium within the fluid reservoir 1530. In operation, a coated medical device 1510 is placed in the fluid reservoir 1530, which is then filled with the first elution medium A. The concentration of the taxane therapeutic agent in the first elution medium A may be detected as a function of time using the detection means 1540. The concentration of the taxane therapeutic agent in the first elution medium A is preferably measured until saturation, for example for a period of about 1-2 hours. After a desired period of time, the first elution medium A can be removed from the fluid reservoir 1530 via the outlet line 1552. The fluid reservoir 1530 may be subsequently filled with the second elution medium B and the concentration of the taxane therapeutic agent in the second elution medium B may be detected by the detection means 1540. Preferably, the second elution medium B is selected to rapidly dissolve any taxane therapeutic agent remaining on the coated medical device 1510 after removing the first elution medium A.

FIG. 18B shows the second device configuration 1505 suitable for obtaining an elution profile in a continuous flow manner. The coated medical device 1510 is placed in a fluid reservoir 1530 configured as a flow-through conduit in the container 1520, permitting an elution medium to flow through the fluid inlet line 1550, contact the coated medical device 1510 and exit the fluid reservoir 1530 by the fluid outlet line 1552 and into the detection means 1540. The elution medium can be changed from a first elution medium A from a first reservoir 1556 to a second elution medium B from a second reservoir 1558 by operating the switch 1554. The second device configuration 1505 operates in the manner of the first device configuration 1500 in FIG. 18A, except that the elution medium flows continuously past the coated medical device and the detection means 1540 can detect the concentration of the taxane therapeutic agent in the elution medium. The detection means 1540 can be include a UV light source 1544, a UV light path 1542 passing through the fluid flow path 1553 to a UV light detector 1546.

The release of taxane therapeutic agent from the medical device can be measured by the detection means 1540 by a suitable spectrographic method, such as measurement of a UV absorption spectrum of the test fluid after contacting the medical device. Any suitable analytical technique(s) may be used to detect a taxane therapeutic agent in an elution medium. Suitable detection methods, such as a spectrographic technique, permit measurement of a property of the elution medium that can be correlated to the presence or concentration of the taxane therapeutic agent with a desired level of accuracy and precision. In one embodiment, absorption spectroscopy can be used to detect the presence of a taxane therapeutic agent, such as in an elution medium. Accordingly, the Beer-Lambert Correlation may be used to determine the concentration of a taxane therapeutic agent in a solution. This correlation is readily apparent to one of ordinary skill in the art, and involves determining the linear relationship between absorbance and concentration of an absorbing species (the taxane therapeutic agent in the elution medium). Using a set of standard samples with known concentrations, the correlation can be used to measure the absorbance of the sample. A plot of concentration versus absorbance can then be used to determine the concentration of an unknown solution from its absorbance.

### Therapeutic Agent Elution Profiles

The composition of a coating comprising a mixture of aPTX and dPTX can be determined by differential elution of each of the solid forms in series. One preferred method of determining the composition of a coating comprises a destructive testing method, whereby a medical device coated with a taxane therapeutic agent is placed in contact with a first elution media, such as porcine serum, that dissolves one solid form of the taxane therapeutic agent at a much faster rate than other solid forms of the taxane therapeutic agent. The presence of the taxane therapeutic agent can be determined by measuring the absorption of the first elution medium at 227 nm, as discussed with respect to FIG. 2. The strength of absorption of the taxane therapeutic agent in the first elution medium can be correlated to the amount of the first solid form of the taxane therapeutic agent in the original coating. Similarly, the amount of absorption in the second elution medium can be correlated to the amount of the second solid form of the taxane therapeutic agent in the original coating. In addition, two stents coated in the same manner can be independently contacted with the first medium or the second medium, and the amount of taxane therapeutic agent elution in each medium can be compared.

For example, porcine serum can be used as a first elution medium to determine the amount of aPTX in a coating. The rate constant for aPTX in porcine serum is about 100-times the rate constant for dPTX in porcine serum. Accordingly, when a medical device coated with a mixture of aPTX and dPTX is placed in a stream of flowing porcine serum, aPTX will elute more rapidly than dPTX, and the downstream absorption of paclitaxel in the elution stream can be correlated to the amount of aPTX in the original coating. The elution medium can be analyzed with HPLC after contacting the coating to quantify the amount of paclitaxel eluted from the coating. SDS may be used as a second elution medium, to rapidly elute the remaining dPTX from the medical device coating. Measuring the amount of paclitaxel in the SDS stream by absorption by HPLC can be correlated to the amount of dPTX in the original coating.

Preferably, the coated medical device can be contacted with a modified porcine serum elution medium at a constant flow rate of 16 mL/min for a desired period of time (e.g., 6-24 hours) sufficient to elute the aPTX present on the stent. The percentage of the taxane therapeutic agent dissolved can be measured as a function of time by monitoring the optical density of the first elution medium at 227 nm after contacting the coated stent, as described above. The modified porcine serum elution medium can be prepared by adding 0.104 mL of a 6.0 g/L Heparin solution to porcine serum at 37° C. and adjusting the pH to 5.6+/−0.3 using a 20% v/v aqueous solution of acetic acid. The elution rate profile of the taxane therapeutic agent can be measured for any desired period, and correlated to the amount of aPTX in the coating. Subsequently, the coated medical device is contacted with a second elution medium comprising 0.3% sodium dodecyl sulfate (SDS) at 25° C. a constant flow rate of 16 mL/min for a suitable time period to elute the dPTX present in the coating. The elution rate profile of the taxane therapeutic agent can be measured for any desired period, and correlated to the amount of aPTX (e.g., by elution in porcine serum) and dPTX (e.g., by subsequent elution in SDS) in the coating.

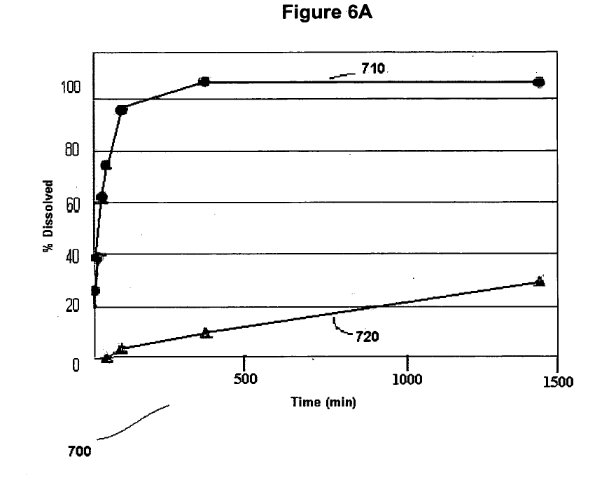

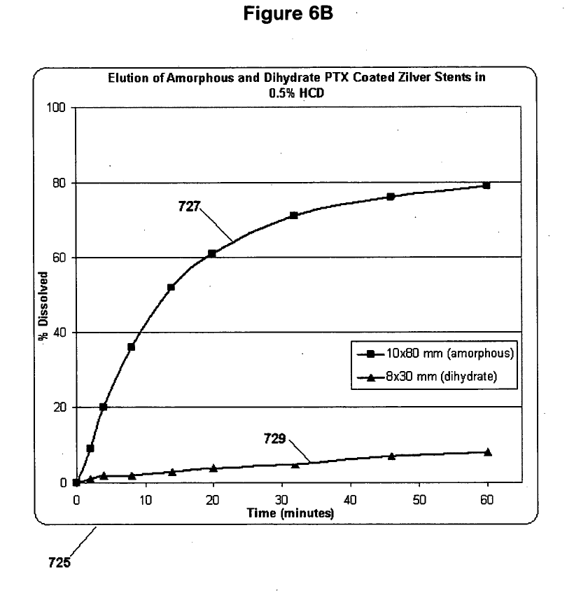

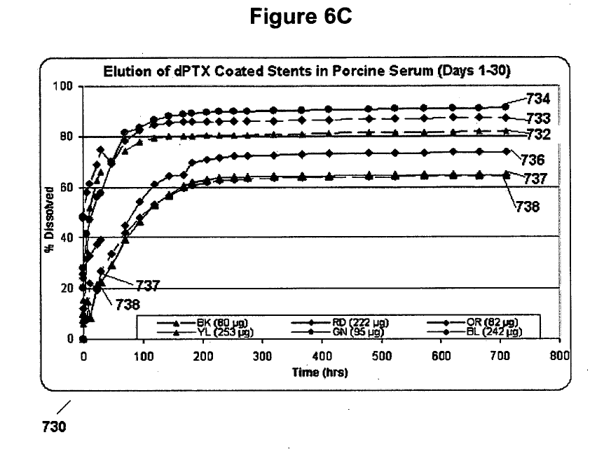

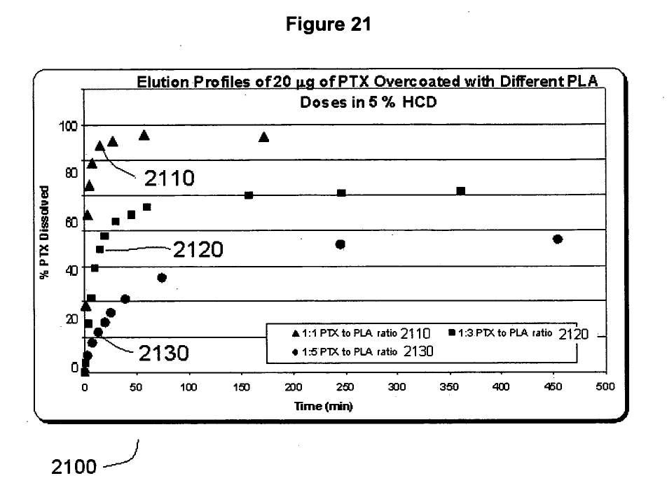

FIG. 6A-6C, FIGS. 7A-7B, FIGS. 19A-19D and FIG. 20 are elution profiles showing the elution of coated vascular stents comprising coatings consisting essentially of paclitaxel in three different elution media (porcine serum, sodium dodecyl sulfate and β-cyclodextrin). The paclitaxel coatings consist of paclitaxel in different solid forms. None of these medical device coatings used to provide these elution profiles contains a polymer. FIG. 21 shows the elution of coated vascular stents comprising a layer of paclitaxel positioned between the abluminal surface of the vascular stent and an overcoat of poly(lactic acid). Each elution profile shows the percentage of the coating dissolved in an elution medium as a function of time the coating is in contact with the elution medium.

The elution profile of taxane therapeutic agent coatings in the amorphous solid form is distinguishable from the elution profile in the solvated (e.g., dihydrate) solid form when porcine serum is used as an elution medium. However, the slow rate of dissolution of the taxane therapeutic agent in the porcine serum can result in undesirably lengthy data collection times to obtain a suitable elution profile. For example, the dihydrate paclitaxel taxane therapeutic agent is less soluble than the amorphous paclitaxel taxane therapeutic agent or the anhydrous paclitaxel taxane therapeutic agent. In porcine serum at 37° C., samples of the dihydrate paclitaxel solid form were about 100-times less soluble than samples of the anhydrous paclitaxel solid form. Other studies have reported decreased solubility of dihydrate paclitaxel in water at 37° C. compared to anhydrous paclitaxel. Anhydrous paclitaxel is reported with a solubility of about 3.5 micrograms/mL after about 5 hours in 37° C. water, while dihydrate paclitaxel has a solubility of less than 1.0 micrograms/mL in 37° C. water over the same time period. R. T. Liggins et al., “Solid-State Characterization of Paclitaxel,” Journal of Pharmaceutical Sciences, v. 86, No. 12, 1458-1463 (December 1997).

FIG. 6A shows elution profiles 700 for two medical devices in porcine serum elution media at 37° C. The first elution profile 710 was obtained from a first coated vascular stent coated with a single layer of amorphous paclitaxel. The second elution profile 720 was obtained from a second coated vascular stent coated with a single layer of dihydrate paclitaxel. The amorphous paclitaxel coating on the first vascular stent had a clear, transparent visual appearance, while the dihydrate paclitaxel coating on the second vascular stent had an opaque, white and cloudy visual appearance. Referring to the first elution profile 710, obtained from the amorphous paclitaxel coating, 100% of the amorphous paclitaxel dissolved within about 6.5 hours (400 minutes), while less than 40% of the second (dihydrate) coating eluted under the same conditions after about 24 hours.

A preferred first elution medium is an aqueous solution comprising 0.1% to about 10% of a cyclodextrin. In one aspect, an elution profile may be obtained by contacting a coated medical device comprising a taxane therapeutic agent with an elution medium comprising a cyclodextrin. A cyclodextrin is a cyclic oligosaccharide formed from covalently-linked glucopyranose rings defining an internal cavity. The diameter of the internal axial cavity of cyclodextrins increases with the number of glucopyranose units in the ring. The size of the glucopyranose ring can be selected to provide an axial cavity selected to match the molecular dimensions of a taxane therapeutic agent. The cyclodextrin is preferably a modified beta-cyclodextrin, such as Heptakis-(2,6-di-O-methyl)-β-cyclodextrin (HCD). Suitable cyclodedtrin molecules include other β-cyclodextrin molecules, as well as γ-cyclodextrin structures.

The elution medium comprising a cyclodextrin can dissolve a taxane therapeutic agent so as to elute the taxane therapeutic agent from a medical device coating over a desired time interval, typically about 24 hours or less (less than comparable elution times in porcine serum). Preferably, the cyclodextrin elution medium is formulated to provide distinguishable elution rates for different coating configurations, providing different elution profiles for different solid forms of a taxane therapeutic agent in the coating. The elution medium may be contacted with a medical device comprising a taxane therapeutic agent, such as paclitaxel, in any manner providing an elution profile indicative of the arrangement of the taxane therapeutic agent molecules in the coating. For example, the elution medium may contact a medical device coating in a continuous flow configuration, or in a batch testing configuration.

Taxane therapeutic agents may have different elution profiles in different elution media. FIG. 6B shows elution profiles 725 for the first and second vascular stents in a 0.5% w/w aqueous solution of Heptakis-(2,6-di-O-methyl)-β-cyclodextrin (HCD) elution medium at 25° C. The first elution profile 727 was obtained from the first coated vascular stent coated with a single layer of amorphous paclitaxel. The second elution profile 729 was obtained from the second coated vascular stent coated with a single layer of dihydrate paclitaxel. Referring to the first elution profile 727, obtained from the amorphous paclitaxel coating, about 80% of the amorphous paclitaxel dissolved within about 1 hour, while less than 20% of the dihydrate paclitaxel was released within 1 hour in the second elution profile 729. Accordingly, comparing FIGS. 6A and 6B, both the HCD and porcine serum elution media selectively dissolved the amorphous paclitaxel distinguishably more rapidly than the dihydrate paclitaxel, however the HCD elution medium dissolved the amorphous paclitaxel much more quickly (727) than the porcine serum (710).

FIG. 6C shows elution profiles 730 for six medical devices in porcine serum elution media at 37° C. for 30 days. All six medical devices were coated with a single layer of paclitaxel in various solid forms, without a polymer or any release-rate-modifying substance. A first elution profile 732, a second elution profile 733 and a third elution profile 734 were obtained coated vascular stents coated with a single layer of about 1 micrograms/mm² (±5%) paclitaxel layer with about 70% of the paclitaxel in the less soluble dihydrate solid form and about 30% of the paclitaxel in the more soluble amorphous solid form. Notably, increasing the total amount of paclitaxel in the single-layer coating from 80 micrograms in the first elution profile 732 to 82 micrograms in the second elution profile 733 to 95 micrograms in the third elution profile 734 resulted in a steady increase in the elution rate. A third elution profile 736, a fifth elution profile 737 and a sixth elution profile 738 were obtained coated vascular stents coated with a single layer of about 3 micrograms/mm² (±15%) paclitaxel layer with about 80% of the paclitaxel in the dihydrate solid form and about 20% of the paclitaxel in the amorphous solid form. Again, increasing the total amount of paclitaxel in the single-layer coating from 222 micrograms in the fourth elution profile 736 to 242 micrograms in the sixth elution profile 738 to 253 micrograms in the fifth elution profile 737 resulted in a steady increase in the elution rate. The rate of elution from the 3 micrograms/mm² paclitaxel coatings was slower than the rate of elution from the 1 micrograms/mm² coatings because the amount of the paclitaxel in the less soluble dihydrate solid form was increased from 70% in the 1 microgram/mm² paclitaxel coatings to 80% in the 3 micrograms/mm² paclitaxel coatings. Accordingly, the rate of release of a paclitaxel coating can be varied by changing the amount of each solid form of the paclitaxel present in a coating. Thus, by varying the solid form of a taxane therapeutic agent, a lower dose of paclitaxel can be used to provide a more sustained release than a higher dose of paclitaxel, without introducing a polymer to the coating.

Taxane therapeutic agents can have different elution profiles in different elution media. Another suitable elution medium for taxane therapeutic agent is sodium dodecyl sulfate (SDS). FIG. 7A shows the solubility of amorphous paclitaxel in sodium dodecyl sulfate (SDS). FIG. 7A is a graph 780 showing a first elution profile 782 obtained from a first coated vascular stent coated with a single layer of amorphous paclitaxel (aPTX) in 0.3% SDS elution medium at 25° C. FIG. 7B shows the solubility of dihydrate paclitaxel in sodium dodecyl sulfate (SDS). FIG. 7B is a graph 790 showing a second elution profile 792 obtained from a second coated vascular stent coated with a single layer of dihydrate paclitaxel (dPTX) in the same 0.3% SDS elution medium at 25° C. The rate of elution of amorphous paclitaxel in the first elution profile 782 is more rapid than the rate of elution of the dihydrate paclitaxel in the second elution profile 792. However, both solid forms of paclitaxel are significantly more soluble in the 0.3% SDS elution medium than in the porcine serum elution media (e.g., compare FIG. 6A and FIGS. 7A-7B).

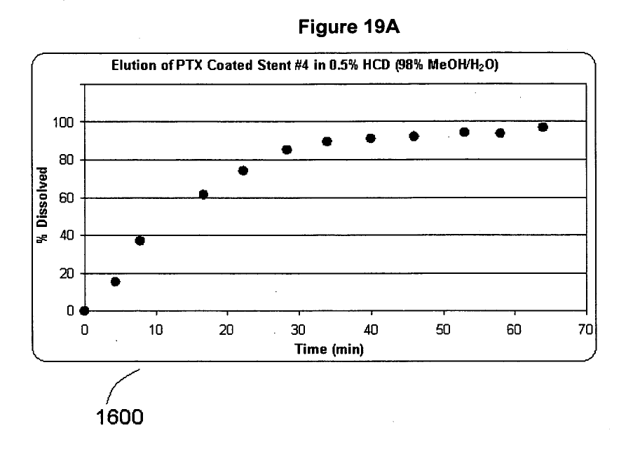

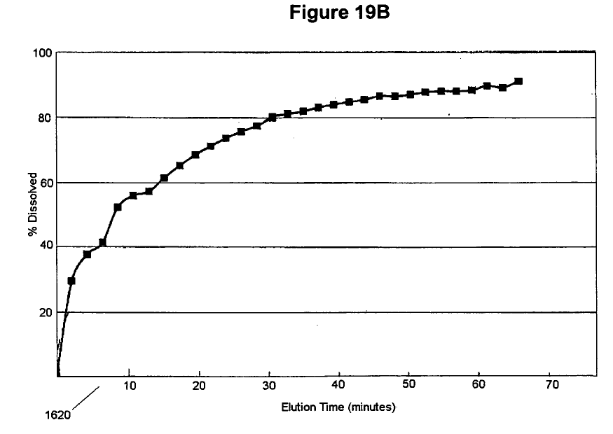

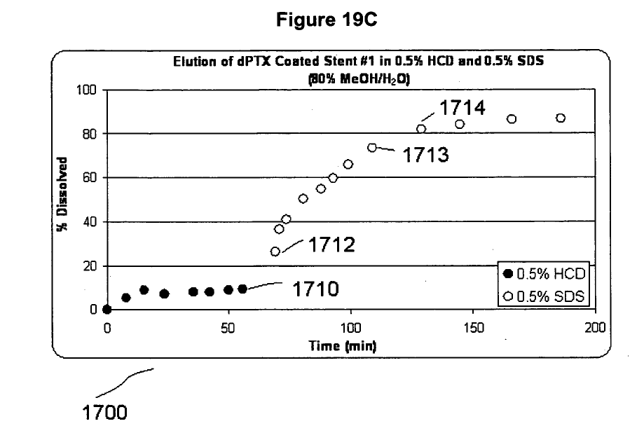

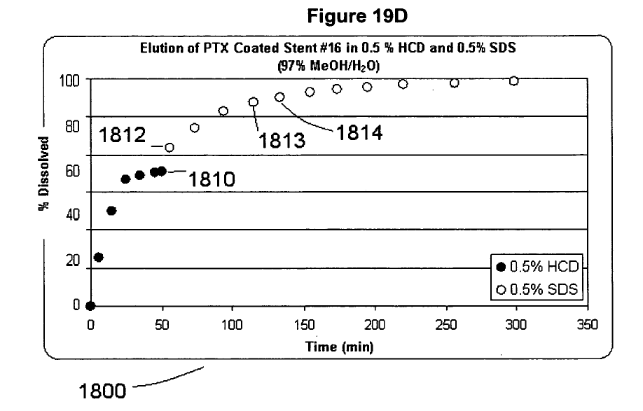

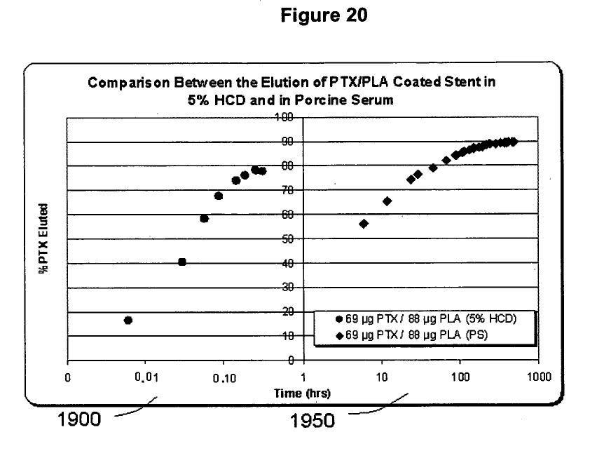

FIG. 19A is an elution profile 1600 of a vascular stent having an amorphous paclitaxel coating obtained in a continuous flow of an aqueous solution of 0.5% HCD cyclodextrin elution medium. The coating did not comprise a polymer. The elution profile in HCD elution medium shows that over 80% of the paclitaxel eluted from the coating after 30 minutes, with the remaining 20% of the coating eluting within the following total 30 minutes. Substantially all amorphous paclitaxel eluting within 1 hour. The amount of paclitaxel in the elution medium was measured by UV absorption at 227 nm.

FIG. 19B is an elution profile 1620 of another vascular stent having an amorphous paclitaxel coating obtained in a continuous flow of an aqueous solution of 0.2% HOD cyclodextrin elution medium. The coating was the same as the coating used to obtain elution profile 1600 (i.e., the coating did not comprise a polymer), but a lower concentration of HOD was used in the elution medium. The elution profile 1620 shows is similar to the elution profile 1600, in that over 80% of the paclitaxel eluted from the coating after 30 minutes, with the remaining 20% of the coating eluting within the following total 30 minutes. Substantially all amorphous paclitaxel eluted within 1 hour in aqueous elution media with 0.2% HOD and 0.5% HOD. The amount of paclitaxel in the elution medium was measured by UV absorption at 227 nm.

FIG. 19C is an elution profile 1700 of a vascular stent having a polymer-free paclitaxel coating obtained with two different elution media. The coating was applied by spraying a solution of paclitaxel in a 80% methanol/20% water solvent system onto a metal stent. First, the coated stent was placed in a continuous flow of an aqueous cyclodextrin elution medium comprising 0.5% HOD as a first elution medium, resulting in elution of about 10% of the coating after 1 hour (data point 1710). The elution profile in HOD elution medium shows a saturation of the percent paclitaxel dissolved from about 45 minutes to the 1 hour data point 1710. Based on the elution profile of amorphous paclitaxel obtained in FIG. 19A (substantially all amorphous paclitaxel eluting within 1 hour), the saturation of paclitaxel solubility by data point 1710 suggests that the coating comprises about 10% of the amorphous paclitaxel solid form. The coated stent was subsequently placed in a continuous flow of a second elution medium comprising an aqueous solution of 0.5% Sodium dodecyl sulfate (SDS), to rapidly elute the remaining paclitaxel from the medical device coating. The amount of paclitaxel in the SDS elution medium was measured by UV absorption at 227 nm. The rate of dissolution in the SDS elution medium increased rapidly from data point 1712 (the first data point indicated that was taken in the SDS elution medium) to data point 1713 (about 40 minutes in the SDS elution medium), and at a slower rate after data point 1714 (about 1 hour in the SDS elution medium), with substantially all of the paclitaxel eluted after 2 hours in the SDS elution medium. The amount of paclitaxel eluted in the SDS elution medium can be correlated to an amount of about 90% dihydrate paclitaxel in the original coating.

FIG. 19D is an elution profile 1800 of a vascular stent having another polymer-free paclitaxel coating obtained with two different elution media. The coating was applied by spraying a solution of paclitaxel in a 97% methanol/3% water solvent system onto a metal stent. The coating included paclitaxel in both the amorphous solid form and the dihydrate solid form. To obtain the elution profile 1800, the coated stent was first placed in a continuous flow of an aqueous cyclodextrin elution medium comprising 0.5% HCD as a first elution medium, resulting in elution of about 70% of the coating after 1 hour (data point 1810). The elution profile in HCD elution medium shows a saturation of the percent paclitaxel dissolved from about 45 minutes to the 1 hour data point 1810. Based on the elution profile of amorphous paclitaxel obtained in FIG. 19A (substantially all amorphous paclitaxel eluting within 1 hour), the saturation of paclitaxel solubility by data point 1810 suggests that the coating comprises about 70% of the amorphous paclitaxel solid form. The coated stent was subsequently placed in a continuous flow of a second elution medium comprising an aqueous solution of 0.5% Sodium dodecyl sulfate (SDS), to rapidly elute the remaining paclitaxel from the medical device coating. The amount of paclitaxel in the SDS elution medium was measured by UV absorption at 227 nm. The rate of dissolution in the SDS elution medium increased rapidly from data point 1812 (the first data point indicated that was taken in the SDS elution medium) to data point 1813 (about 40 minutes in the SDS elution medium), and at a slower rate after data point 1814 (about 1 hour in the SDS elution medium), with substantially all of the paclitaxel eluted after 2 hours in the SDS elution medium. The amount of paclitaxel eluted in the SDS elution medium can be correlated to an amount of about 30% dihydrate paclitaxel in the original coating.

### Coatings Comprising Release Modifying Agents

In one aspect, methods of detecting the elution of a taxane therapeutic agent may be detected from a medical device coating comprising a taxane therapeutic agent that is substantially free of a polymer, or contains less than about 0.50 micrograms, 0.10 micrograms or 0.05 micrograms of a polymer per mm² of abluminal surface area and preferably less than 10 micrograms, 5 micrograms, 1 micrograms or 0.5 micrograms of a polymer total in the coating. Most preferably, the coating is free of a polymer, or contains less than about 0.50 micrograms, 0.10 micrograms or 0.05 micrograms of any polymer per mm² of abluminal surface area and preferably less than 10 micrograms, 5 micrograms, 1 micrograms or 0.5 micrograms of any polymer total in the coating.

In another aspect, the elution of a taxane therapeutic agent from a medical device coating comprising the taxane therapeutic agent and a release modifying agent that modifies the release of the therapeutic agent, such as a polymer or protein. Such a coating may include two or more coating layers each comprising or consisting essentially of a taxane therapeutic agent in one or more solid forms. Preferred multilayer coatings include an outer layer comprising an amorphous solid form of a taxane therapeutic agent. The outer layer preferably covers the exposed surface of the underlying coating layer(s). The outer layer can optionally include a mixture of other solid forms of the taxane therapeutic agent with the amorphous solid form. Multilayer coatings can include any number of coating layers beneath the outer coating, including 2, 3, 4, 5, 6, 7, and 8-layer coatings. One preferred two-layer coating configuration includes a first layer consisting essentially of a dihydrate paclitaxel solid form, and a second layer comprising an amorphous paclitaxel solid form. The second layer can be a mixture of the amorphous and the dihydrate solid forms of paclitaxel.

Testing methods provided herein comprise the step of contacting a medical device known to comprise both a releasable taxane therapeutic agent and a release modifying agent with an elution medium comprising a cyclodextrin and detecting the taxane therapeutic agent in the elution medium. This method can provide an elution profile of the coated medical device that may change due to changes in the coating configuration, such as the ratio of the release modifying agent to the taxane therapeutic agent or the number of composition of coating layers. Such methods are useful in performing lot release testing of medical devices by comparing the elution profile of a coating being tested with the elution profile of a standard medical device coating of a desired configuration. A suitable elution medium can be any solvent system in which a desired medical device coating configuration has an elution profile that can be distinguished from the elution profile of a different, undesirable medical device coating configuration. A lot release criteria for evaluating a medical device coating may require that the elution profile of the taxane therapeutic agent from a medical device coating tested be sufficiently similar to the elution profile of a standard sample known to contain the desired solid form of the taxane therapeutic agent. Standard samples of the taxane therapeutic agent can be prepared and characterized in bulk form, and the elution profile of each solid form can be obtained in a suitable elution medium.

In a first aspect, the release modifying agent is a polymer, such as a biodegradable polymer or a biostable polymer. The polymer can cover the therapeutic agent coated on a surface of a medical device, such as a stent, or can be mixed with the therapeutic agent in one or more layers. The coating can be applied to any suitable surface of a medical device, including on substantially flat or roughened metal surfaces, impregnation within tissue grafts or polymer gels, within grooves, holes or wells formed in portions of a device. The medical device is preferably configured as a vascular stent or stent graft, although the coatings can be applied to any suitable implantable medical device. For example, implantable portions of catheters, billiary or urological stents or shunts, stent grafts, tissue grafts, orthopedic implants, pacemakers, implantable valves and other implantable devices can be coated with the coatings disclosed herein, so as to release a therapeutic agent upon implantation.

In one coating configuration, the polymer release modifying agent is a biodegradable polymer, preferably a bioabsorbable elastomer. Examples of suitable biodegradable polymer include a polyhydroxyalkanoate compound, a hydrogel, poly(glycerol-sebacate), an elastin-like peptide, a polyhydroxyalkanoate bioabsorbable polymer such as polylactic acid (poly lactide) (PLA), polyglycolic acid (poly glycolide) (PGA), polylactic glycolic acid (poly lactide-co-glycolide) (PLGA), poly-4-hydroxybutyrate, polyanhydrides, polyorthoesters or a combination of any of these. Biodegradable polymers can have different rates of dissipation upon implantation within a body, and can be selected based on the intended use of the medical device. PLA coatings can be formulated as simi-crystalline (L-isomer) or amorphous (D-isomer), and are absorbed slowly upon implantation (about 5 years). PGA polymer coating can provide a semi-crystalline structure, a stronger acid than PLA, a more readily hydrolyzed in situ than PLA and dissipation within the body within about 1-3 months. Polylactic-co-glycolic acid (PLGA) is the product of the copolymerization of PLA and PGA. By varying the PLA/PGA ratio, the properties of the copolymer can be controlled. Features of preferred biodegradable coating polymers are summarized in Table 3 below.

TABLE 3

| | | Degradation Rate | | Typical |
|---|---|---|---|---|
| Polymer | Crystallinity | (a) | | Applications |
| PGA | High Crystallinity | 2-3 | months | Suture, soft |
| | | | | anaplerosis |
| PLLA | Semi-crystalline | >2 | years | Fracture fixation, |
| | | | | ligament |
| PDLA | Amorphous | 12-16 | months | Drug delivery |
| | | | | system |
| PLGA | Amorphous | 1-6 | months (b) | Suture, fracture |
| | | | | fixation, oral |
| | | | | implant, drug |
| | | | | delivery |

(a) Rate depends on molecular rate of polymer
(b) Rate depends on the ratio of LA and GA

The release modifying agent may also be a biostable polymer, which can be configured as a porous layer mixed with and/or deposited over a layer comprising the taxane therapeutic agent. Preferably, the polymers used in the coating are selected from the following: styrene-isobutylene-styrene copolymers, polyurethanes, silicones (e.g., polysiloxanes and substituted polysiloxanes), and polyesters. Other polymers which can be used include ones that can be dissolved and cured or polymerized on the medical device. Still other polymers that may be used include ultraviolet cross-linkable polymers and/or high temperature setting thermoses polymers. Additional suitable polymers include thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene monomer) rubbers, fluorosilicones, polyethylene glycol, polysaccharides, phospholipids, and combinations of the foregoing. Hydrogel polymers such as polyhema, polyethylene glycol, polyacrylamide, and other acrylic hydrogels may also be used. Other hydrogel polymers that may be used are disclosed in U.S. Pat. No. 5,304,121, U.S. Pat. No. 5,464,650, U.S. Pat. No. 6,368,356, PCT publication WO I 95/03083 and U.S. Pat. No. 5,120,322, which are incorporated by references.

FIG. 20 and FIG. 21 are elution profile graphs showing the elution rates of comparable medical device coatings comprising paclitaxel and a biodegradable polymer in two different solvents (porcine serum and β-cyclodextrin). To obtain the data for both FIG. 20 and FIG. 21, the amount of paclitaxel eluted was determined by monitoring the characteristic peak of paclitaxel at 227 nm by UV detection within the elution media after contacting the medical device coating, as described above.

FIG. 20 shows a first elution profile 1900 and a second elution profile 1950 both obtained from two substantially identical coated vascular stents, each comprising a two-layer coating with a first layer of paclitaxel deposited on the outer surface of the stent and a second layer of PLA deposited over and enclosing the first layer of paclitaxel. The first coating layer on each coated stent included a total of 69 micrograms of paclitaxel, covered by a total of 88 micrograms of PLA. The first elution profile 1900 was obtained by contacting the first coated stent with a continuous flow of an aqueous elution medium with 5% HCD, while the second elution profile 1950 was obtained by placing the second coated stent in a continuous flow of porcine serum. The coating eluted much more rapidly in the HCD cyclodextrin elution medium than the porcine serum elution medium. In the first elution profile 1900, about 70% of the paclitaxel eluted after about 0.1 hours (6 minutes), and about 80% of the coating eluted within about 1 hour. In contrast, in the second elution profile 1950, less than 60% of the paclitaxel eluted after about 6 hours, less than 70% after about 10 hours, and nearly 100 hours were required to elute 90% of the paclitaxel. Accordingly, the use of porcine serum as an elution medium can require extended testing periods to ascertain the elution profile of paclitaxel from a coating comprising a polymer and paclitaxel, while substantially less time may be required to obtain comparable data when using a cyclodextrin elution medium.

FIG. 21 shows a set of three elution profiles 2100 obtained from substantially identical coated vascular stents having similar two-layer PLA-paclitaxel coatings, but differing in the ratio of PLA to paclitaxel in the coating. All three coatings have a first layer of 20 micrograms paclitaxel applied to the exterior surface of substantially identical vascular stents, and a second layer of PLA applied over and enclosing the first layer. The coatings differed in the amount of PLA in the second layer. All three elution profiles 2100 were obtained by placing the coated stents in a continuous flow of an aqueous solution of 5% HCD cyclodextrin elution medium. The first elution profile 2110 was obtained from a coating having 20 micrograms of PLA (a paclitaxel:PLA mass ratio of 1:1) (shown as triangular data points), and eluted most rapidly of the three coatings. The second elution profile 2120 was obtained from a coating having 60 micrograms of PLA (a paclitaxel:PLA mass ratio of 1:3) (shown as square data points), and eluted more slowly than the coated stent of the first elution profile 2110. The third elution profile 2140 was obtained from a coating having 100 micrograms of PLA (a paclitaxel:PLA mass ratio of 1:5) (shown as circular data points), and eluted the most slowly of the three elution profiles 2100.

Increasing the amount of PLA relative to the amount of paclitaxel decreased the elution rate of the paclitaxel in cyclodextrin elution medium. Referring to Example 8 below, elution of similar two-layer coatings of PLA over paclitaxel in porcine serum also demonstrate an increase in the elution time of paclitaxel as the amount of PLA is increased. The coatings eluted in Example 8, like the second elution profile 1950 in FIG. 20, also required extended times of over 100 hours to elute up to about 70% to 90% of the paclitaxel, depending on the amount of PLA. Such lengthy elution times can be disadvantageous in obtaining lot release data.

In another aspect, the release modifying agent is a protein, such as zein. Zein refers to a group of prolamine proteins present in maize seed. During development of the maize kernel, zein accretions form in the peripheral regions of the lumen of the rough endoplasmin reticulum. These ultimately develop into cytoplasmic deposits called vesicular protein bodies ranging in size from 1 to 3 μm in diameter. Various methods and techniques exist for extracting zein from the maize endosperm. Laboratory preparation of zein, for example, involves extracting zein from maize endosperm with aqueous ethanol or isopropanol under mild conditions (such as an extraction temperature less than 10 Celsius) with or without reducing agents. Commercial zein is typically extracted from corn gluten meal. For example, U.S. Pat. Nos. 3,535,305, 5,367,055, 5,342,923, and 5,510,463 disclose extraction of zein from corn gluten using aqueous-alcohol solutions. Commercial zeins include Wako Pure Chemical Industries product numbers 261-00015, 264-01281, and 260-01283; Spectrum Chemical product numbers Z1131 and ZE105; ScienceLab stock keeping unit SLZ1150; SJZ Chem-Pharma Company product name ZEIN (GLIDZIN); and Arco Organics catalog numbers 17931-0000, 17931-1000, and 17931-5000; and product number Z 3625, zein from maize, obtained from Sigma-Aldrich, St. Louis, Mo.

Zein proteins include three types of proteins: α-zein, γ-zein (which includes β-zein), and δ-zein. These can be further differentiated into four classes (α-, β-, γ-, and δ-) on the basis of differences in solubility and sequence. Zein extracted without reducing agents forms a large multigene family of polypeptides, termed α-zein. The other fractions of zein (β-, γ-, and δ-zein) may be extracted using aqueous alcohols containing reducing agents to break disulfide bonds. For example, mercaptoethanol is used for laboratory extraction. γ-Zein is soluble in both aqueous and alcoholic solvents with reducing conditions. γ-Zein typically comprises about 10 to 15% of total zein proteins, β-Zein constitutes up to 10% of the total zein and δ-Zein is a minor fraction of zein. δ-Zeins are the most hydrophobic of the group. Zein proteins are considered as Generally Recognized as Safe (G.R.A.S.) by the Food and Drug Administration since 1985 (CAS Reg. No. 9010-66-6).

The medical device coating may comprise a taxane therapeutic agent and zein in one or more layers. For example, the coating may have two layers: a first layer consisting of the paclitaxel, or comprising a mixture of zein and paclitaxel, may be covered or enclosed by a second layer consisting of zein or paclitaxel alone, or a mixture of zein and paclitaxel. The second layer may serve as a barrier that slows the rate of release of the taxane therapeutic agent from the underlying first layer by providing an additional layer through which the taxane therapeutic agent must diffuse or by providing an additional layer that must degrade before releasing the therapeutic agent beneath it. Preferably, at least a portion of the abluminal surface of the medical device has a layer of admixed therapeutic agent and zein. The zein may function to increase the biocompatibility of the medical device, and the presence of a therapeutic agent on the abluminal surface of the device allows the release of the agent directly to the location in need of therapy.

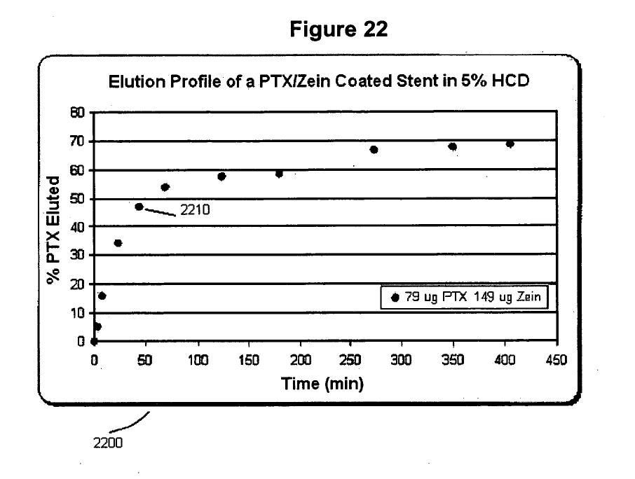

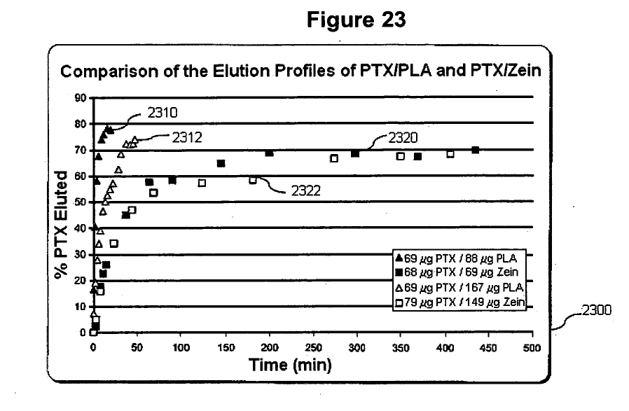

FIG. 22 and FIG. 23 are elution profile graphs showing the elution rates of medical device coatings comprising paclitaxel and a Zein release modifying agent in a HCD β-cyclodextrin elution medium. To obtain the data for both FIG. 22 and FIG. 23, the amount of paclitaxel eluted in the elution medium was determined by monitoring the characteristic peak of paclitaxel at 227 nm by UV detection within the elution media after contacting the medical device coating, as described above.

FIG. 22 shows a graph 2200 of drug elution in an aqueous solution of 5% HCD from a two-layer paclitaxel-zein coated stent. The stent is coated on the abluminal surface, with a first layer of paclitaxel covered by a second layer of zein positioned over the first layer. The stent is s tubular vascular stent having a cylindrical lumen defined by a luminal interior surface and an abluminal exterior surface. The elution of therapeutic agent is indicated as a percentage by weight of total drug initially deposited on the stent. Typical units for drug elution include micrograms of drug. The zein-coated stent elution rate profile 2210 was obtained from a stent coated only on the abluminal surface with 79 μg of paclitaxel in a first layer covered with 149 μg of zein in a second layer.

FIG. 23 is a graph comparing elution rate profiles 2300 for vascular stents coated with a first layer of paclitaxel covered by a second layer of either polylactic acid (PLA) (profiles 2310, 2312) or zein (profiles 2320, 2322). Elution rate profile 2310, obtained from a first coated stent coated with 69 μg of paclitaxel in a first layer covered by 88 μg of PLA in a second layer, shows the fastest rate of elution of the four elution profiles 2310, 2312, 2320 and 2322. The elution profile 2312 shows a comparably slower rate of drug delivery and was obtained from a second coated stent coated with 69 μg of paclitaxel in a first layer covered by 167 μg of PLA in a second layer. Using the 5% aqueous HCD elution medium, the elution profile 2312 obtained from the second coated stent was distinguishable from the elution profile 2310 obtained from the first coated stent, where the first coating contained an equal amount of paclitaxel but about half the amount of PLA in the coating compared to the second coating. Comparably slower elution profiles were obtained by replacing the PLA release modifying agent with zein stents. The elution rate profile 2320, obtained from a third stent coated with 68 μg of paclitaxel in a first layer covered by 69 μg of zein in a second layer, was substantially slower drug than the elution profiles 2310 or 2312. Using the 5% aqueous HCD elution medium, the elution profile 2320 obtained from the zein-coated stent was distinguishable from the elution profile 2310 obtained from the first coated stent. The elution rate profile 2322 was obtained from a fourth coated stent coated with 79 μg of paclitaxel in a first layer and 149 μg of zein in a second layer.

### Kinetic Parameters for Elution of a Therapeutic Agent Coating

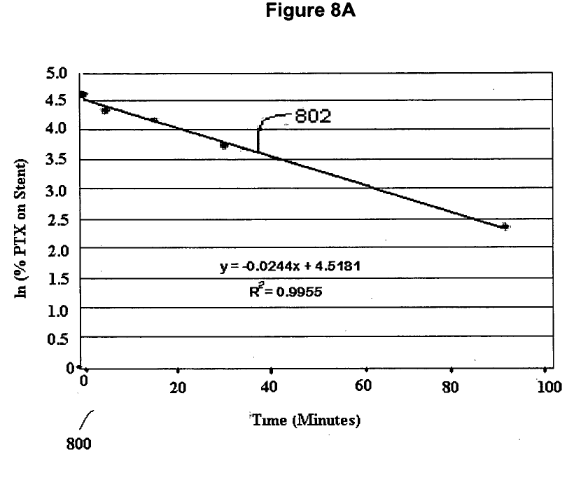

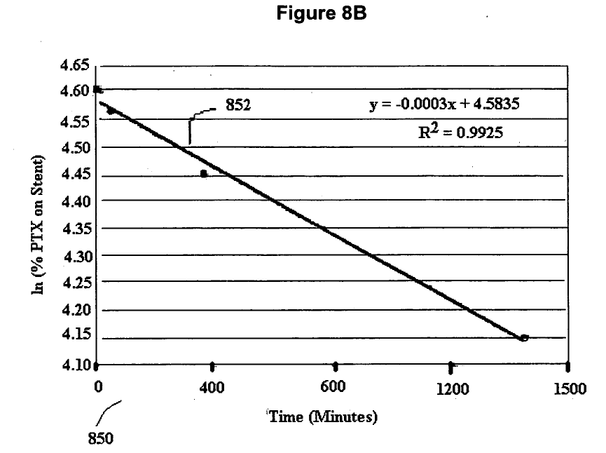

The release of a therapeutic agent from a coating may be estimated by measuring the elution of the therapeutic agent in an elution medium. The rate constant for the release of a therapeutic agent in a coating configuration may be determined, and an estimated rate of elution as a function of coating composition may be obtained. FIG. 8A shows a first-order kinetic plot 800 of the data from the first elution profile 710 in FIG. 6A. The first kinetic plot 800 plots the natural log of the percent of the amorphous paclitaxel coating remaining on the first vascular stent as a function of time (minutes). The data in the first kinetic plot 800 closely fits to straight line 802 (R²=0.9955), indicating that the elution of amorphous paclitaxel in porcine serum at 37° C. follows first order kinetics. Based on the slope of the line 802, the first order rate constant of amorphous paclitaxel in porcine serum (37° C.) is about 0.0244 min⁻¹, with a half life of about 30 minutes.

Similarly, FIG. 8B shows a first-order kinetic plot 850 of the data from the second elution profile 720 in FIG. 6A. The kinetic plot 850 indicates the natural log of the percent of the dihydrate paclitaxel coating remaining on the second vascular stent as a function of time (minutes). The data in the first kinetic plot 850 also closely fits to straight line 852 (R²=0.9925), indicating that the elution of dihydrate paclitaxel in porcine serum at 37° C. also follows first order kinetics. Based on the slope of the line 852, the first order rate constant of dihydrate paclitaxel in porcine serum (37° C.) is about 0.0003 min⁻¹, with a half life of about 38.5 hours (2,310 minutes). Therefore, the rate of elution of the amorphous paclitaxel is about 100-times faster than dihydrate paclitaxel in porcine serum (37° C.).

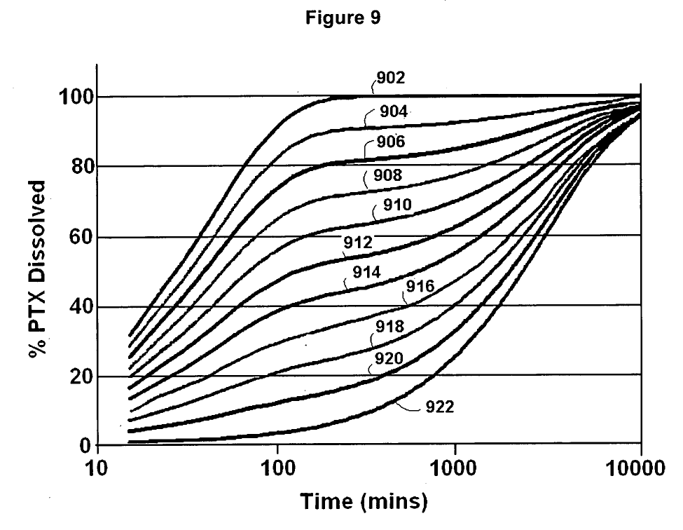

Based on the first order rate constants obtained for amorphous paclitaxel (k₁=0.0244 min⁻¹) and for dihydrate paclitaxel (k₂=0.0003 min⁻¹), the rate of dissolution of a coating comprising of a mixture of amorphous and dihydrate taxane therapeutic agents can be formulated as a function of the proportion of each solid form by the formulae: f=1−(ae^{k}₁^{t}+(1−a)e^{k}₂^{t}) and a=(1−f−e^{k}₂^{t})/e^{k}₁^{t}−(e^{k}₂^{t}), where f is the fraction dissolved, k₁ and k₂ are the rate constants for amorphous and dihydrate paclitaxel respectively, a is the proportion of amorphous taxane therapeutic agent in the coating layer, (1−a) is the amount of dihydrate taxane therapeutic agent in the coating layer and e is the natural logarithmic base. FIG. 9 shows a plot of the predicted dissolution of a mixture of amorphous paclitaxel and dihydrate paclitaxel having the first order rate constants k₁ and k₂ respectively as a function of time and composition. A first trace 904 corresponds to the predicted dissolution profile of a coating comprising 10% amorphous paclitaxel (aPTX) and 90% dihydrate paclitaxel (dPTX). The composition corresponding to the traces of FIG. 9 is provided in Table 4 below. The percentage of the paclitaxel dissolved as a function of time for about 1 week (10,000 minutes) is shown for each trace.

TABLE 4
Compositions of predicted elution profiles shown in FIG. 8

| Trace in FIG. 9 | Percentage aPTX | Percentage dPTX |
|---|---|---|
| 902 | 100 | 0 |
| 904 | 90 | 10 |
| 906 | 80 | 20 |
| 908 | 70 | 30 |
| 910 | 60 | 40 |
| 912 | 50 | 50 |
| 914 | 40 | 60 |
| 916 | 30 | 70 |
| 918 | 20 | 80 |
| 920 | 10 | 90 |
| 922 | 0 | 100 |

Preferably, the conditioning step(s) increase the amount of a hydrated solid form (such as the dihydrate solid form) within the coating. Accordingly, the conditioning step(s) may change the composition of a taxane therapeutic coating from a pre-conditioning composition represented by any composition corresponding to traces 902-920 to a composition represented by a higher-numbered trace. For example, a pre-conditioned coating consisting essentially of paclitaxel in mixture of solid forms corresponding to trace 906 may be conditioned prior to implantation to provide a coating corresponding to the slower-eluting trace 918. Varying the relative amounts of amorphous and dihydrate paclitaxel in the coating by conditioning can result in wide variation of the rate of release of paclitaxel from the coating. Referring again to FIG. 9, after about 1-2 hours (100 minutes), less than 10% of the dihydrate paclitaxel coating (922) has dissolved, while about 80% of the amorphous paclitaxel coating (902) has dissolved. Mixtures of amorphous and dihydrate paclitaxel (904-920) can show intermediate amounts of elution. Similarly, after about 16 hours (1,000 minutes), less than 30% of the dihydrate paclitaxel coating (922) has dissolved, about 100% of the amorphous paclitaxel coating (902) has dissolved and mixtures of amorphous and dihydrate paclitaxel (904-920) can show intermediate amounts of elution. Finally, after about 1 week (10,000 minutes), about 90-95% of the dihydrate paclitaxel coating (922) has dissolved, with mixtures of amorphous and dihydrate paclitaxel (904-920) showing nearly 100% elution.

The elution profiles of coatings modeled by the traces of FIG. 9 correspond to coatings having a taxane therapeutic agent distributed in a mixture of multiple solid forms within the coating, most preferably a coating formed from a mixture of amorphous state paclitaxel and a solvated (e.g., dihydrate) solid form paclitaxel. A coating having a mixture of the amorphous and taxane therapeutic agent solid forms can be prepared as described above with respect to the third embodiment.

The dihydrate paclitaxel taxane therapeutic agent is also less soluble than the amorphous taxane therapeutic agent or the anhydrous taxane therapeutic agent. In porcine serum at 37° C., samples of the dihydrate paclitaxel solid form were about 100-times less soluble than samples of the anhydrous paclitaxel solid form. Other studies have reported decreased solubility of dihydrate paclitaxel in water at 37° C. compared to anhydrous paclitaxel. Anhydrous paclitaxel is reported with a solubility of about 3.5 μg/mL after about 5 hours in 37° C. water, while dihydrate paclitaxel has a solubility of less than 1.0 μg/mL in 37° C. water over the same time period. R. T. Liggins et al., “Solid-State Characterization of Paclitaxel,” Journal of Pharmaceutical Sciences, v. 86, No. 12, 1458-1463 (December 1997).

### Preparation of Taxane Therapeutic Agent Coating Standards

The elution profiles obtained from lot release testing of coated medical devices having unknown coating compositions may be compared to elution profiles obtained from standard medical device coatings prepared with known compositions, such as a paclitaxel coating having a known ratio of amorphous solid form to dihydrate solid form. By comparing the elution profile from the standard having a known coating composition, differences in the composition of the unknown sample for lot release testing may be identified. Accordingly, lot release testing criteria may be determined by comparison of comparable elution profiles for coated medical devices with elution profiles obtained from standard coated medical devices with known coating compositions.

For example, a lot release testing method may include coating a medical device with a taxane therapeutic agent to form a standard coated medical device in compliance with at least one lot testing criterion. The lot testing criterion may be any measurable quality of the coating, such as the visual appearance, a spectroscopic determination (e.g., a vibrational spectrum, 13C NMR spectrum, XRD spectrum) that is indicative of the physical structure of the coating, or a physical property such as melting point or solubility. Preferably, the lot testing criteria is based at least in part on the solubility of the coating in an elution medium. Accordingly, the lot release criteria may be satisfied by obtaining an elution profile for a sample coated medical device and comparing the elution profile with a comparable elution profile independently obtained for a standard coated medical device. Acceptable criteria, such as percent variation between the two elution profiles, may be established to determine whether the lot is acceptable by meeting the lot testing criterion or criteria. The lot release testing method may also include the steps of: contacting the standard coated medical device with a first elution medium comprising a cyclodextrin for a first period of time; measuring the taxane therapeutic agent in the first elution medium as a function of time the standard coated medical device is in contact with the elution medium to obtain a standard elution profile; selecting a sample coated medical device including a taxane therapeutic agent from a first lot of coated medical devices; contacting the sample coated medical device with a second elution medium comprising a cyclodextrin for a second period of time; measuring the taxane therapeutic agent in the second elution medium as a function of time the sample coated medical device is in contact with the elution medium to obtain a sample elution profile; and comparing the first elution profile with the second elution profile to determine whether the sample coated medical device meets the at least one lot testing criterion. Preferably, the first time period and the second time period are less than 24 hours, and more preferably less than 18, 12, 10, 8, 7, 6, 5, 4, 3, 2 or 1 hour(s). The first time period and second time period may be selected to provide a desired amount of elution of the therapeutic agent from the sample coated medical device or the standard coated medical device. For example, the first period of time may be substantially equal to the second period of time and is less than about 12 hours. The first elution medium and the second elution medium may each comprise an aqueous solution comprising between about 0.1% and 10% Heptakis-(2,6-di-O-methyl)-β-cyclodextrin, including amounts of 0.2, 0.3, 0.4, 0.5, 0.6, 0.7, 0.8, 0.9, 1.0, 5.0, 8.0 and 10.0% HCD in water at 25° C.

In one example, the lot release testing method further comprises the steps of: contacting the standard coated medical device with a third elution comprising sodium dodecyl sulfate after contacting the standard medical device with the first elution medium comprising a cyclodextrin; detecting the taxane therapeutic agent in the third elution medium; contacting the sample coated medical device with a fourth elution comprising sodium dodecyl sulfate after contacting the standard medical device with the second elution medium comprising a cyclodextrin; and detecting the taxane therapeutic agent in the fourth elution medium.

Medical device coatings can comprise one or more of the solid forms of the taxane therapeutic agents, and may be provided by spray coating a taxane therapeutic agent spray coating solution onto a surface of a medical device in any suitable manner, such as a coating method described herein. For example, the coating may also be deposited onto the medical device by spraying, dipping, pouring, pumping, brushing, wiping, vacuum deposition, vapor deposition, plasma deposition, electrostatic deposition, epitaxial growth, or any other method known to those skilled in the art. Preferably, however, the medical device coatings are applied by spraying methods, such as those described herein.

Spray coating methods are preferably used to deposit taxane therapeutic agents onto the surface(s) of a medical device in one or more different solid forms. The spray coating can be performed by any suitable coating technique, but typically includes the step of dissolving the taxane therapeutic agent in a suitable solvent and spraying the resulting solution onto the surface of the medical device. Changing the solvent(s) in the solution can change the solid forms of the resulting taxane therapeutic agent deposited on a medical device. To deposit a coating of a dihydrate taxane therapeutic agent, a recrystallized dihydrate taxane therapeutic agent from the first embodiment can be dissolved in a suitable organic alcohol solvent, such as methanol. To deposit a coating layer comprising a mixture of dihydrate and amorphous taxane solid forms, the taxane is preferably dissolved in a spray solvent comprising a mixture of water and a protic solvent such as methanol. Importantly, varying the ratio of water to methanol and/or the concentration of the taxane in the spray solvent comprising the taxane typically changes the composition of the resulting coating layer that is spray deposited. Generally, increasing the amount of methanol in the spray solution results in a coating layer with a higher proportion of amorphous taxane.

Preferred spray solutions for obtaining durable coating are also listed herein, along with the preferred resulting minimum ratio of dihydrate to amorphous solid forms obtained by ultrasonic spray coating of the preferred solution. Importantly, the ratio of amorphous to dihydrate solid forms in a solid taxane solid coating may be changed by altering the methanol to water ratio and/or the concentration of the taxane therapeutic agent in the spray solution. Decreasing the concentration of the taxane in the spray solution may require a lower methanol to water ratio (i.e., less methanol and more water by volume) to obtain a given dihydrate to amorphous ratio in the solid coating formed after spraying and evaporation of the solvent. The spray solution can be made with any suitable concentration of the taxane therapeutic agent, although concentrations of about 0.5-5 mM are preferred, with concentrations of about 4.68 mM, 2.34 mM, 1.74 mM, 1.17 mM or 0.70 mM being particularly preferred. The relationship between the concentration of the taxane therapeutic agent in the spray solution, the ratio of methanol to water in the spray solution and the ratio of dihydrate to amorphous solid forms in the solid coating formed by spray coating the spray solution is illustrated with respect to paclitaxel in Tables 5a and 5b. Table 5a provides preferred spray solvent compositions for the spray deposition of a coating layer comprising a mixture of dihydrate paclitaxel and amorphous paclitaxel using a 4.68 mM paclitaxel concentration in the spray solution. Table 5a shows the ratio of methanol to water in a spray coating solution comprising about 4.68 mM paclitaxel, and the ratio of amorphous:dihydrate paclitaxel in a single coating layer deposited on a stent surface by spray coating the solutions with the specified compositions. Table 5b shows the ratio of methanol and water in a spray solution comprising various two-solvent solutions at 2.34 mM paclitaxel, 1.74 mM paclitaxel and 0.70 mM paclitaxel. Preferably, the coatings were applied by spraying a solution of 1.74 mM paclitaxel

TABLE 5a
Spray Coating Solvent Compositions
for 4.68 mM Paclitaxel Solution

| dPTX:aPTX ratio | Solvent (% MeOH:H₂0) |
|---|---|
| >90%:<10% | 60:40%-90:10% |
| 60:40%-70:30% | 92:8%-93.5:6.5% |
| 40:60%-50:50% | 93.5:6.5%-94.55.5% |
| 30:70%-40:60% | 95:5%-97.5:2.5% |

TABLE 5b
Spray Coating Solvent Compositions
at Lower Paclitaxel Concentrations

| | Solvent | |
|---|---|---|
| dPTX:aPTX ratio | (% MeOH:H₂0) | [PTX] mM |
| 52:48% | 88:12% | 2.34 |
| 42:58% | 90:10% | |
| 25:75% | 93:7% | |
| 78:22% | 70:30% | 0.70 |
| 65:35% | 75:25% | |
| 55:45% | 80:20% | |

In one aspect, the amount of hydrated solid form of a taxane therapeutic agent is increased by applying an additional layer of the taxane therapeutic agent to an existing coating of the taxane therapeutic agent. Increasing the number of spray applications of the 1.74 mM paclitaxel solution increased the amount of dihydrate paclitaxel solid form at a given methanol to water ratio. As shown in Table 5c, applying each of two 1.74 mM paclitaxel solutions in a methanol-water binary solvent system (a first solution consisting of 68% methanol and 32% water or a second solution consisting of 65% methanol and 35% water) by spray coating resulted in higher fractions of dihydrate paclitaxel solid form after multiple spray coating applications (e.g., passes of the spray gun over the surface) than a single application.

TABLE 5c
Multiple Spray Applications of a Paclitaxel Solution

| | Solvent | |
|---|---|---|
| dPTX:aPTX ratio | (% MeOH:H₂0) | [PTX] mM |
| 33:67 (1 application) | 68:32% | 1.74 |
| 60:40 (4 applications) | 68:32% | |
| 34:66 (1 application) | 65:35% | |
| 39:61 (4 applications) | 65:35 | |

In addition to selecting an appropriate solvent system, other coating parameters such as the spraying apparatus, spray rate, and nozzle configuration can be selected to provide coatings comprising one or more solid forms of a taxane therapeutic agent. Preferably, the taxane therapeutic agent is spray coated onto a medical device surface using an ultrasonic spray deposition (USD) process. Ultrasonic nozzles employ high frequency sound waves generated by piezoelectric transducers which convert electrical energy into mechanical energy. The transducers receive a high frequency electrical input and convert this into vibratory motion at the same frequency. This motion is amplified to increase the vibration amplitude at an atomizing surface.

Ultrasonic nozzles are typically configured such that excitation of a piezoelectric crystal creates a longitudinal standing wave along the length of the nozzle. The ultrasonic energy originating from the transducers may undergo a step transition and amplification as the standing wave traverses the length of the nozzle. The nozzle is typically designed such that a nodal plane is located between the transducers. For ultrasonic energy to be effective for atomization, the nozzle tip must be located at an anti-node, where the vibration amplitude is greatest. To accomplish this, the nozzle's length should be a multiple of a half-wavelength. In general, high frequency nozzles are smaller, create smaller drops, and consequently have smaller maximum flow capacity than nozzles that operate at lower frequencies.

Liquid introduced onto the atomizing surface absorbs some of the vibrational energy, setting up wave motion in the liquid on the surface. For the liquid to atomize, the vibrational amplitude of the atomizing surface should be adequately controlled. Below a certain amplitude, the energy may be insufficient to produce atomized drops. If the amplitude is excessively high, cavitation may occur. The input power is preferably selected to provide an amplitude producing a desired spray having a fine, low velocity mist. Since the atomization mechanism relies largely on liquid being introduced onto the atomizing surface, the rate at which liquid is atomized depends on the rate at which it is delivered to the surface.

For example, the medical device may be coated using an ultrasonic spray nozzle, such as those available from Sono-Tek Corp., Milton, N.Y. The spray solution can be loaded into a syringe, which is mounted onto a syringe pump and connected to a tube that carries the solution to the ultrasonic nozzle. The syringe pump may then used to purge the air from the solution line and prime the line and spay nozzle with the solution. The stent may be loaded onto a stainless steel mandrel in the ultrasonic coating chamber. The stent may optionally be retained around a mandrel during coating. Alternatively, the stent may be secured and rotated on a clip or in within a steam of rapidly flowing gas such as nitrogen. Preferably, contact between the stent and the mandrel is minimized so as to prevent a “webbed” coating between struts. Typically, the luminal surface is not coated although the coating may be applied to any surface, if desired.

The medical device may be a vascular stent mounted around a mandrel. The mandrel may be fastened onto a motor, positioned below the ultrasonic nozzle. The motor rotates the mandrel at a pre-set speed and translationally moves the stent underneath the ultrasonic spray. In one aspect, the rotational speed is set to 10 rpm and the translational speed is set to 0.01 mm per second. In another aspect, the rotational speed is set to 60 rpm and the translational speed is set to 0.05 mm per second. In yet another embodiment, the rotational speed is set to 30-150, preferably about 110 rpm, and the translational speed is set to 0.19 mm per second. Other speeds and combinations may also be used in the present invention. Preferred coating parameters for USD using a Sono-tek Model 06-04372 ultrasonic nozzle are provided in Table 6 below:

TABLE 6
Ultrasonic Spray Deposition Parameters
for Sono-tek Model 06-04372

| Flow | Coating | Rotation | Nozzle | Process | |
|---|---|---|---|---|---|
| rate | velocity | Speed | Power | Gas | Distance |
| (mL/min) | (in/sec) | (rpm) | (watts) | (psi) | (mm) |
| 0.01-2 | 0.01-0.5 | 30-150 | 0.9-1.2 | 0.1-2.5 | 1-25 |

Importantly, ultrasonic spray coating is preferably performed at an ambient temperature of about 85-87° F. and in a coating chamber at a pressure of less than about 0.05 psi. The temperature is preferably selected to provide a desirably uniform, solvent-free coating. Preferably, the coating is performed at a temperature of about 60-90° F., preferably about 85-87° F. The quality of the coating may be compromised if coating is performed outside the preferred temperature range. The temperature during ultrasonic spray coating should be high enough to rapidly evaporate the methanol in the spray solution before contacting the stent (i.e., at least about 80° F.).

Most preferably, the ultrasonic spray coating is performed at a flow rate of about 0.03 mL/min, a coating velocity of about 0.025 in/sec, a rotation speed of about 60 rpm, a nozzle power of about 1 watt, a process gas pressure of about 2 psi, a distance of about 12 mm between the nozzle and medical device, and a temperature of about 85° F. within a coating chamber. The coating chamber is purged with nitrogen to displace oxygen in the system. During the process, the stent is kept at ambient temperature and in a closed chamber.

To obtain the desired dosage of therapeutic agent, the solid form of the taxane therapeutic agent in the coating may be varied. In one embodiment, the coating contains from about 0.01 micrograms to about 10 micrograms of the taxane therapeutic agent per mm² of the surface area of the structure, preferably about 0.05 micrograms to about 5 micrograms, about 0.03 micrograms to about 3 micrograms, about 0.05 micrograms to about 3 micrograms, about 0.5 micrograms to about 4.0 micrograms, most preferably between about 0.5 and 3.0 micrograms, of the taxane therapeutic agent per mm2 of the abluminal surface area of the structure. Desirably, a total of about 1-500 micrograms of a taxane therapeutic agent (such as paclitaxel) is coated on one or more surface of a medical device.

Notably, as the dose of paclitaxel in the coating increases, more amorphous solid form is typically needed to maintain a given level of durability. For example, a paclitaxel-only coating having a 50:50 ratio of the dihydrate:amorphous solid forms was durable at a dose of 3 micrograms/mm2 but not for a dose of 1 micrograms/mm2. That is, paclitaxel coatings with less than 50% dihydrate solid form were typically required to maintain durability at the 1 micrograms/mm2 coating that was comparable to the 3 micrograms/mm2 coating.

Table 7 below provides examples of preferred abluminal paclitaxel coatings on a 6×20 radially expandable vascular stent, showing the relationship between the composition of the spray solution and the resulting coating composition. Each coating is deposited using ultrasonic deposition according to Table 6 above at a temperature of about 87° F. The spray solution included the concentration of paclitaxel in Table 7 with methanol and water in a ratio that provides a desired amount of the dihydrate solid form. As described by Table 5a and Table 5b, increasing the amount of methanol relative to water resulted in less dihydrate in the coating at any concentration of paclitaxel.

TABLE 7
Preferred Paclitaxel Coatings

| | | | Concentration |
|---|---|---|---|
| | | Preferred | Paclitaxel |
| Paclitaxel Dose | Total Paclitaxel | dPTX:aPTX for | in Spray |
| (micrograms/mm²) | (micrograms) | durability (%:%) | Solution (mM) |
| 0.06 | 5 | 80:20 | 0.70 |
| 0.30 | 24 | 75:25 | 1.74 |
| 1.00 | 74 | 70:30 | 2.34 |
| 3.00 | 219 | 50:50 | 4.68 |

The thickness of the coating layer comprising the taxane therapeutic agent is between 0.1 micrometer and 20 micrometers, between 0.1 micrometer and 10 micrometers, or between 0.1 micrometer and 5 micrometers. For the purposes of local delivery from a stent, the daily dose that a patient will receive depends at least on the length of the stent. The total coating thickness is preferably about 50 micrometers or less, preferably less than about 20 micrometers and most preferably about 0.1-10 micrometers.

For example, a 6×20 mm stent may be coated with about 0.05-5 micrograms/mm² of paclitaxel, more preferably about 0.5-3 micrograms/mm², can be applied to the abluminal surface of the stent. Particularly preferred doses of a taxane therapeutic agent on the abluminal surface of a stent include: 0.06, 0.30, 1.00 and 3.00 micrograms/mm². In another embodiment, the abluminal side of a 6×20 mm stent (surface area of about 73 mm²) is coated with about 20-220 micrograms of paclitaxel. Examples of particularly preferred coatings for a 6×20 mm vascular stent having an abluminal surface area of about 73 mm2, and a compressed diameter of about 7 F.

The coated medical devices may be sterilized prior to implantation into the body, including before and/or after coating. Preferably, the coated medical device is sterilized using a conventional chemical vapor sterilization process that does not undesirably degrade or alter the taxane therapeutic coating. For example, a conventional ethylene oxide (ETO) sterilization process may be used, which may involve exposing the coated medical device to ETO gas at a temperature of about 120° F. for at least a period suitable for sterilizing the medical device. Since ethylene oxide gas readily diffuses through many common packaging materials and is effective in killing microorganisms at temperatures well below those required for heat sterilization techniques, ETO sterilization can permit efficient sterilization of many items, particularly those made of thermoplastic materials, which cannot withstand heat sterilization. The process generally involves placing an item in a chamber and subjecting it to ethylene oxide vapor. When used properly, ethylene oxide is not only lethal to microorganisms, but it is also non-corrosive, readily removed by aeration.

Notably, the ratio of dihydrate to amorphous solid forms of the taxane therapeutic agent may increase during ETO sterilization. For example, increases of up to about 5% in the proportion of dihydrate paclitaxel were observed in coatings consisting of paclitaxel in both the dihydrate and amorphous solid forms prior to sterilization. Typically, coated medical devices can be sterilized within suitable packaging, such as a bag, pouch, tube or mold.

Alternatively, the medical device may be loaded into final packaging, and gamma irradiated in a gamma chamber. In one embodiment, the implantable medical device is irradiated with between 1 and 100 kGy. In another embodiment, the implantable medical device is irradiated with between 5 and 50 kGy, and in yet another embodiment, the implantable medical device is irradiated with between 25 and 28 kGy.

The coatings preferably comprise a taxane therapeutic agent with a desired level of durability for an intended use. Coating durability describes the resistance of a coating to loss of integrity due to abrasion, bending or mechanical loading through mechanisms such as flaking, cracking, chipping and the like. Coatings consisting of dihydrate taxane therapeutic agents demonstrated a low durability, and a high propensity for dissociation from the stent coating upon crimping. In contrast, the amorphous solid form of the taxane therapeutic agents demonstrated greater durability and substantially lower tendency to dissociate from a coated stent upon crimping of the stent. In aqueous media such as porcine serum and blood, the amorphous taxane therapeutic agent solid form is more soluble than the dihydrate taxane therapeutic agent.

The durability of a coating can be measured by weighing a coated medical device prior to physical agitation of the coating by a test process such as crimping, shaking, freezing or abrading the stent, weighing the coated stent a second time after the test process is performed, and comparing the second weight to the first weight. For a given physical test procedure, coating durability can be quantified by the amount of weight loss from the first weight to the second weight. Accordingly, the lower the amount of weight loss as a result of performing a physical test on the coated medical device, the more durable the coating is. One preferred physical test for implantable coated vascular stents is the process of crimping the stent from an expanded state (in which the stent is coated), to a radially compressed state for delivery within a body vessel. The durability of a radially expandable medical device can be quantified as the percentage weight loss of the coated medical device before and after crimping the medical device.

The difference in weight of a coated stent before and after crimping provides one indicator of the coating durability. Preferably, the coated medical device is crimped into a radially compressed state prior to implantation within a body vessel. Highly durable coatings typically have a lower weight loss during the crimping process. Taxane coatings with a higher proportion of dihydrate are typically less durable (i.e., higher weight loss during the crimping process). Preferred taxane coatings exhibit a coating weight loss of less than about 10%, more preferably less than about 8%, 6%, 4%, 3%, 2%, 1% or 0.5% and most preferably less than about 0.1% before and after crimping to a diameter of 6 French (6 F). The coating weight loss can be measured by: (1) weighing an uncoated stent in the radially expanded state to obtain a first weight (“weight (1)”), (2) coating the stent in the expanded state, (3) weighing the coated stent to obtain a second weight (“weight (2)”), (4) crimping the coated stent and (5) weighing the crimped, coated stent to obtain a third weight (“weight (3)”). The coating weight loss is: [weight (2)−weight (1)]−[weight (3)−weight (1)], or simply weight (2)−weight (3). Accordingly, one particularly preferred coating comprises a mixture of amorphous taxane therapeutic agent and dihydrate taxane therapeutic agent. Coatings comprising mixtures of dPTX with at least about 25-50% aPTX on the outside surface of the coating have shown desired durability characteristics.

Particularly preferred coatings applied with a 4.68 mM paclitaxel solution comprise about 30% aPTX and 70% dPTX. A stent comprising a 30:70 aPTX:dPTX was coated in a radially expanded state, crimped to fit a delivery catheter, and re-weighed. This 30:70 aPTX:dPTX coated stent lost less than 5% weight as a result of crimping to a 6 F size.

The durability of the coating may also be evaluated as the resistance to displacement of the coating in response to mechanical abrasion. For instance, scraping a non-durable coating may displace a portion of the coating from one area to another, resulting in a scratching or pitting of the surface without a net change in the weight of the coating. Preferably, coatings are sufficiently durable to resist displacement by mechanical abrasion as well as weight loss. Preferred coatings have a substantially uniform and smooth surface. Most preferably, coatings maintain a surface roughness (peak to valley) that is less than 50%, preferably 25%, of the total thickness of the coating. For instance, for a 10 micrometer thick coating, the surface is preferably not more than about 5 micrometers from its highest peak to its lowest valley. Also preferably, the coating roughness does not increase as a result of mechanical abrasion of a type encountered in crimping and loading the coated medical device into a delivery catheter.

### Medical Devices

The coatings may be applied to one or more surfaces of any implantable medical device having any suitable shape or configuration. The medical device may be adapted or selected for temporary or permanent placement in the body for the prophylaxis or treatment of a medical condition. The present invention is applicable to implantable or insertable medical devices of any shape or configuration. Typical subjects (also referred to herein as “patients”) are vertebrate subjects (i.e., members of the subphylum cordata), including, mammals such as cattle, sheep, pigs, goats, horses, dogs, cats and humans.

Sites for placement of the medical devices include sites where local delivery of taxane therapeutic agents are desired. Common placement sites include the coronary and peripheral vasculature (collectively referred to herein as the vasculature). Other potential placement sites include the heart, esophagus, trachea, colon, gastrointestinal tract, biliary tract, urinary tract, bladder, prostate, brain and surgical sites, particularly for treatment proximate to tumors or cancer cells. Where the medical device is inserted into the vasculature, for example, the therapeutic agent is may be released to a blood vessel wall adjacent the device, and may also be released to downstream vascular tissue as well.

The medical device of the invention may be any device that is introduced temporarily or permanently into the body for the prophylaxis or therapy of a medical condition. For example, such medical devices may include, but are not limited to, stents, stent grafts, vascular grafts, catheters, guide wires, balloons, filters (e.g. vena cava filters), cerebral aneurysm filler coils, intraluminal paving systems, sutures, staples, anastomosis devices, vertebral disks, bone pins, suture anchors, hemostatic barriers, clamps, screws, plates, clips, slings, vascular implants, tissue adhesives and sealants, tissue scaffolds, myocardial plugs, pacemaker leads, valves (e.g. venous valves), abdominal aortic aneurysm (AAA) grafts, embolic coils, various types of dressings, bone substitutes, intraluminal devices, vascular supports, or other known biocompatible devices.

In general, intraluminal stents for use in connection with the present invention typically comprise a plurality of apertures or open spaces between metallic filaments (including fibers and wires), segments or regions. Typical structures include: an open-mesh network comprising one or more knitted, woven or braided metallic filaments; an interconnected network of articulable segments; a coiled or helical structure comprising one or more metallic filaments; and, a patterned tubular metallic sheet (e.g., a laser cut tube). Examples of intraluminal stents include endovascular, biliary, tracheal, gastrointestinal, urethral, ureteral, esophageal and coronary vascular stents. The intraluminal stents of the present invention may be, for example, balloon-expandable or self-expandable. Thus, although certain embodiments of the present invention will be described herein with reference to vascular stents, the present invention is applicable to other medical devices, including other types of stents.

In one embodiment of the present invention, the medical device comprises an intraluminal stent. FIG. 16 shows a coated medical device comprising a self-expanding vascular stent 10 having a luminal surface 12 and a coating 37 applied to the abluminal surface 14. The vascular stent 10 extends from a proximal end 13 to a distal end 15. The vascular stent 10 has a tubular shape formed from a series of joined hoops 16 formed from interconnected struts 17 and bends 18, and defines the interior lumen. The stent may be self-expanding or balloon-expandable and may be a bifurcated stent, a coronary vascular stent, a urethral stent, a ureteral stent, a biliary stent, a tracheal stent, a gastrointestinal stent, or an esophageal stent, for example. More specifically, the stent may be, for example, a Wallstent, Palmaz-Shatz, Wiktor, Strecker, Cordis, AVE Micro Stent, Igaki-Tamai, Millenium Stent (Sahajanand Medical Technologies), Steeplechaser stent (Johnson & Johnson), Cypher (Johnson & Johnson), Sonic (Johnson & Johnson), BX Velocity (Johnson & Johnson), Flexmaster (JOMED) JoStent (JOMED), S7 Driver (Medtronic), R-Stent (Orbus), Tecnic stent (Sorin Biomedica), BiodivYsio (Abbott), Trimaxx (Abbott), DuraFlex (Avantec Vascular), NIR stent (Boston Scientific), Express 2 stent (Boston Scientific), Liberte stent (Boston Scientific), Achieve (Cook/Guidant), S-Stent (Guidant), Vision (Guidant), Multi-Link Tetra (Guidant), Multi-Link Penta (Guidant), or Multi-Link Vision (Guidant). Some exemplary stents are also disclosed in U.S. Pat. Nos. 5,292,331 to Boneau, 6,090,127 to Globerman, 5,133,732 to Wiktor, 4,739,762 to Palmaz, and 5,421,955 to Lau. Desirably, the stent is a vascular stent such as the commercially available Gianturco-Roubin FLEX-STENT®, GRII™, SUPRA-G, ZILVER or V FLEX coronary stents from Cook Incorporated (Bloomington, Ind.).

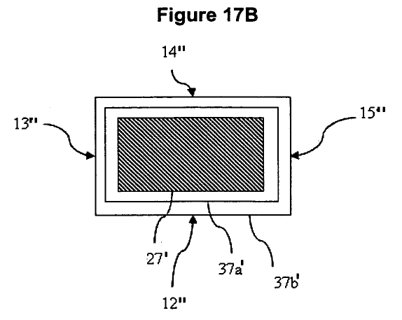

FIG. 17A shows a cross section along line A-A′ of coated strut 17′ from the vascular stent 10 shown in FIG. 16. Referring to FIG. 17A, the strut 17′ can have any suitable cross sectional configuration, such as a rectangular cross section, and can be formed from any suitable material 27 such as a nickel titanium alloy, stainless steel or a cobalt chromium alloy. The abluminal surface 14′, including the proximal edge 13′ and distal edge 15′, are coated with the coating 37 adhered to the abluminal surface of the vascular stent 10. Preferably, the coating 37 includes one or more solid forms of a taxane therapeutic agent, such as paclitaxel. In one aspect, the coating 37 can consist essentially of a single solid form of the taxane therapeutic agent, such as a dihydrate solvated paclitaxel. In another aspect, the coating 37 includes a single layer comprising a mixture of two or more solid forms of the taxane therapeutic agent, such as a mixture of dihydrate solvated paclitaxel and amorphous paclitaxel. In yet another aspect, the coating 37 can include two or more coating layers each comprising one or more solid forms of the taxane therapeutic agent. Each coating layer may be distinguished, for example, by different elution rates resulting from different solid form structure(s) in each layer. The coating 37 can also include non-taxane components, such as biostable or bioabsorbable polymers, in separate layers from or combined with a taxane therapeutic agent. FIG. 17B shows an alternative cross-sectional view of the portion A-A′ of the medical device strut 17′ shown in FIG. 16.

Referring to FIG. 17B, the strut 27′ can have any suitable cross sectional configuration, such as a rectangular cross section, and can be formed from any suitable material such as a nickel titanium alloy, stainless steel or a cobalt chromium alloy. The abluminal surface 14″, including the proximal edge 13″ and distal edge 15″, are coated with a two layer coating including a first layer 37a′ and a second layer 37b′. The coating is adhered to the abluminal surface of the vascular stent 10. Preferably, the first layer 37a′ of the coating includes one or more solid forms of a taxane therapeutic agent, such as paclitaxel. The second layer 37b′ may include a release modifying agent, such as a porous material, a biodegradable material, or other component adapted to alter the rate of elution of the therapeutic agent. The coating can also include non-taxane components, such as biostable or bioabsorbable polymers, in separate layers from, or combined with, a taxane therapeutic agent. Alternatively, the first layer 37a′ may include the release modifying agent and the second layer 37b′ may include the taxane therapeutic agent.

For restenosis treatment, it is desirable that the release be initiated before or at the time at which cell proliferation occurs, which generally begins approximately three days after the injury to the artery wall by the PTCA procedure. Of course, the release profile will be tailored to the condition that is being treated. For example, where an anti-inflammatory or anti-thrombotic effect is desired, release is typically initiated sooner. Moreover, in instances where DNA is used that has an expression half-life that is shorter than the time period desired for administration of the therapy, release of the DNA from the device is typically regulated such that it occurs over a time period longer than the half-life of the DNA expression, thus allowing new copies of DNA to be introduced over time and thereby extending the time of gene expression.

The stent or other medical device of the invention may be made of one or more suitable biocompatible materials such as stainless steel, nitinol, MP35N, gold, tantalum, platinum or platinum iridium, niobium, tungsten, inconel, ceramic, nickel, titanium, stainless steel/titanium composite, cobalt, chromium, cobalt/chromium alloys, magnesium, aluminum, or other biocompatible metals and/or composites or alloys such as carbon or carbon fiber. Other materials for medical devices, such as drainage stents or shunts, include cellulose acetate, cellulose nitrate, silicone, cross-linked polyvinyl alcohol (PVA) hydrogel, cross-linked PVA hydrogel foam, polyurethane, polyamide, styrene isobutylene-styrene block copolymer (Kraton), polyethylene terephthalate, polyurethane, polyamide, polyester, polyorthoester, polyanhydride, polyether sulfone, polycarbonate, polypropylene, high molecular weight polyethylene, polytetrafluoroethylene, or other biocompatible polymeric material, or mixture of copolymers thereof; polyesters such as, polylactic acid, polyglycolic acid or copolymers thereof, a polyanhydride, polycaprolactone, polyhydroxybutyrate valerate or other biodegradable polymer, or mixtures or copolymers thereof; extracellular matrix components, proteins, collagen, fibrin or other therapeutic agent, or mixtures thereof. Desirably, the device is made of stainless steel, cobalt-chromium or a nickel-titanium alloy (e.g., Nitinol).

The stent may be deployed according to conventional methodology, such as by an inflatable balloon catheter, by a self-deployment mechanism (after release from a catheter), or by other appropriate means. The stent may be formed through various methods, such as welding, laser cutting, or molding, or it may consist of filaments or fibers that are wound or braided together to form a continuous structure. The stent may also be a grafted stent in which the therapeutic agent is incorporated into the graft material.

### Methods of Treatment

Methods of treatment preferably include the step of inserting into a patient a coated medical device having any of the compositions and/or configurations described above. For example, when the medical device is a stent coated by the coating methods described above, the method of treatment involves implanting the stent into the vascular system of a patient and allowing the therapeutic agent(s) to be released from the stent in a controlled manner, as shown by the drug elution profile of the coated stent. Optionally, a method of treatment may further comprise the steps of obtaining a first elution profile from a standard coated medical device of known coating composition and comparing the first elution profile with a second elution profile obtained from a second coated medical device selected as a representative sample from a first lot of similarly manufactured coated medical device. If the second elution profile is sufficiently similar to the first elution profile, another coated medical device from the first lot may be selected (i.e., a “selected coated medical device”) and implanted within a body vessel as described below to treat a condition. The selected coated medical devices may be subsequently implanted to treat peripheral vascular disease, for example by implanting the coated medical device in a peripheral artery. In one aspect, methods of treating peripheral vascular disease (PVD) are provided. PVD is a disease of the lower extremities that may present various clinical indications ranging from asymptomatic patients, to patients with chronic critical limb ischemia (CLI) that might result in amputation and limb loss.

Methods of treating peripheral vascular disease, including critical limb ischemia, preferably comprise the endovascular implantation of one or more conditioned and coated medical devices provided herein. Atherosclerosis underlies many cases of peripheral vascular disease, as narrowed vessels that cannot supply sufficient blood flow to exercising leg muscles may cause claudication, which is brought on by exercise and relieved by rest. As vessel narrowing increases, critical limb ischemia (CLI) can develop when the blood flow does not meet the metabolic demands of tissue at rest. While critical limb ischemia may be due to an acute condition such as an embolus or thrombosis, most cases are the progressive result of a chronic condition, most commonly atherosclerosis. The development of chronic critical limb ischemia usually requires multiple sites of arterial obstruction that severely reduce blood flow to the tissues. Critical tissue ischemia can be manifested clinically as rest pain, nonhealing wounds (because of the increased metabolic requirements of wound healing) or tissue necrosis (gangrene).

The coated medical device can be implanted in any suitable body vessel. Typical subjects (also referred to herein as “patients”) are vertebrate subjects (i.e., members of the subphylum cordata), including, mammals such as cattle, sheep, pigs, goats, horses, dogs, cats and humans. Sites for placement of the medical devices include sites where local delivery of taxane therapeutic agents are desired. Common placement sites include the coronary and peripheral vasculature (collectively referred to herein as the vasculature). Other potential placement sites include the heart, esophagus, trachea, colon, gastrointestinal tract, biliary tract, urinary tract, bladder, prostate, brain and surgical sites, particularly for treatment proximate to tumors or cancer cells. Where the medical device is inserted into the vasculature, for example, the therapeutic agent is may be released to a blood vessel wall adjacent the device, and may also be released to downstream vascular tissue as well.

The configuration of the implantable frame can be selected based on the desired site of implantation. For example, for implantation in the superficial artery, popliteal artery or tibial artery, frame designs with increased resistance to crush may be desired. For implantation in the renal or iliac arteries, frame designs with suitable levels of radial force and flexibility may be desired. Preferably, a coated vascular stent is implanted in a non-coronary peripheral artery, such as the iliac or renal arteries.

In one embodiment, a medical device comprising a balloon-expandable frame portion coated with a taxane therapeutic agent can be endoluminally delivered to a point of treatment within an infrapopliteal artery, such as the tibial or peroneal artery or in the iliac artery, to treat CLI. For treating disease conditions, coated balloon-expandable medical devices can comprise an expandable frame attached to a coating. The frame can be also be formed from a bioabsorbable material, or comprise a coating of the therapeutic agent material over at least a portion of the frame. The frame can be configured to include a barb or other means of securing the medical device to the wall of a body vessel upon implantation.

In another aspect, a coated medical device can be a self-expanding device such as a coated NITINOL stent coated with the taxane therapeutic agent, and configured to provide a desirable amount of outward radial force to secure the medical device within the body vessel. The medical device can be preferably implanted within the tibial arteries for treatment of CLI. For instance, the coated medical device can be configured as a vascular stent having a self-expanding support frame formed from a superelastic self-expanding nickel-titanium alloy coated with a metallic bioabsorbable material and attached to a graft material. A self-expanding frame can be used when the body vessel to be stented extends into the distal popliteal segment. The selection of the type of implantable frame can also be informed by the possibility of external compression of an implant site within a body vessel during flexion of the leg.

In one aspect, methods of delivering a therapeutic agent to a blood vessel are provided. The methods may include the step of providing a coated vascular stent comprising a radially-expandable vascular stent having an abluminal side and a luminal side defining a substantially cylindrical lumen and being movable from a radially expanded configuration to a radially compressed configuration; and a coating on at least one surface of the vascular stent. The coating may include a taxane therapeutic agent such as paclitaxel in one or more solid forms. Preferably, the coating includes paclitaxel in the dihydrate solid form. The method may also include the steps of: intralumenally inserting the coated vascular stent into the blood vascular system using a means for intralumenal delivery comprising a catheter, positioning the coated vascular stent within a peripheral artery; and radially expanding the coated vascular stent within the peripheral artery so as to place the coated vascular stent in contact with a portion of a wall of the peripheral artery in a manner effective to deliver the therapeutic agent to the wall of the blood vessel.

A consensus document has been assembled by clinical, academic, and industrial investigators engaged in preclinical interventional device evaluation to set forth standards for evaluating drug-eluting stents such as those contemplated by the present invention. See “Drug-Eluting Stents in Preclinical Studies—Recommended Evaluation From a Consensus Group” by Schwartz and Edelman (available at “http://www.circulationaha.org” (incorporated herein by reference).

### EXAMPLES

In the following examples, the equipment and reagents specified below were used:

TABLE 8
Reagents and Equipment

| | | Manufacturer | |
|---|---|---|---|
| Equipment Name | Manufacturer | ID | Vendor |
| 1 μg Balance | Mettler | AX 26 | VWR |
| 10 μg Balance | Mettler | AX 105 DR | VWR |
| Top Loading Balance | Ohaus | GT 4100 | VWR |
| | | (not avail.) | |
| Inline Spectrometer | Agilent | 8453 | Agilent |
| Chemstation | Agilent | Version A, | Agilent |
| | | 10.01 | |
| Coating Spectrometer | Perkin Elmer | Lambda 14 P | Perkin Elmer |
| 1 | | | |
| Coating Spectrometer | Perkin Elmer | Lambda 45 | Perkin Elmer |
| 2 | | | |
| UV Winlab | Perkin Elmer | Version 5.1 | Perkin Elmer |
| Cuvettes | Perkin Elmer | B0631077 | VWR |
| Electrostatic Coater | Terronics | Custom | Terronics |
| MED Spray | Badger | Model 200 | Ding-A-Ling |
| Gun/Badger | | | |
| Cook Incorporated | EFD | 780S-SS | EFD |
| Spray Gun | | | |
| Cook Incorporated | EFD | Valvemate | EFD |
| Spray Controller | | 7040 | |
| Microscope | Leica | MZ-16 | Nuhsbaum |
| | | | Inc. |
| Image Pro Plus | MediaCybernetics | Version 5.1 | Media |
| | | | Cybernetics |
| Microsoft Office | Microsoft | Version 2003 | New Egg |
| Stopwatch | Private Label | n/a | VWR |
| Glassware | Kimball | Various | VWR |
| Ethanol | Aaper | E 200 PP | Aaper |
| Methanol | Sigma | M3641 | Sigma |
| DicHloromethane | Sigma | 15,479-2 | Sigma |
| Water | Ricca Chemical | 9150-5 | VWR |

### Example 1

### Preparation of Amorphous, Anhydrous and Dihydrate Paclitaxel

Bulk samples of amorphous, anhydrous and dihydrate paclitaxel solid forms were prepared by the methods described below. These preparations were reproduced based on Jeong Hoon Lee et al., “Preparation and Characterization of Solvent Induced Dihydrate, Anhydrous and Amorphous Paclitaxel,” Bull. Korean Chem. Soc. v. 22, no. 8, pp. 925-928 (2001).

Samples of bulk amorphous paclitaxel were prepared as follows: 1.01 g of paclitaxel (Phytogen Life Sciences) was dissolved in 5 mL dichloromethane (Mallinckrodt) while agitating to form a paclitaxel solution; the paclitaxel solution was left open to air at about 23° C. for about 10 hours to permit evaporation of the dichloromethane and formation of amorphous paclitaxel. The melting temperature of the amorphous paclitaxel was 209-215° C.

Samples of bulk anhydrous paclitaxel were prepared as follows: 1.06 g of paclitaxel (Phytogen Life Sciences) were dissolved in 40 mL methanol (Sigma Aldrich, 99.93% HPLC Grade) while sonnicating the container and inversion of the container to form a paclitaxel solution; about 2 mL of hexane (Sigma Aldrich) was added to the paclitaxel solution, and the solution was placed in a freezer at about −20° C. overnight (approximately 10 hours) to form anhydrous crystalline paclitaxel. The melting temperature of the anhydrous paclitaxel was 190-210° C.

Samples of dihydrate paclitaxel were prepared as follows: 1.09 g paclitaxel (Phytogen Life Sciences) were dissolved in 25 mL methanol while sonnicating the container to form a paclitaxel solution; about 5 mL of water was added to the paclitaxel solution; and the sample was placed in a freezer at about −20° C. overnight to form dihydrate crystals. The melting temperature of the dihydrate crystal was 209-215° C. Subsequently, the sample was sealed under vacuum to 0.025 torr for 2.5 hours to remove residual solvent. Dihydrate paclitaxel samples were also prepared as follows: 50.08 g paclitaxel (Phytogen Life Sciences) was dissolved in 1.1 L methanol to form a solution; 275 mL water was subsequently added to the methanol solution in a drop-wise fashion to form a precipitate that was refrigerated at about −20° C. overnight (about 10 hours); the resulting solid precipitate was filtered, dissolved in 1500 mL methanol and 375 mL water and was subsequently added in a drop-wise fashion; the resulting crystals were recrystallized a third time using 1200 mL methanol with 300 mL water; and the resulting dihydrate crystals were collected.

### Example 2

### Ultraviolet (UV) Spectra of Bulk Paclitaxel Samples

The three solid samples prepared in Example 1 (amorphous, dihydrate and anhydrous paclitaxel) were dissolved in ethanol to form spray sample solutions. The ultraviolet spectra of each of the three samples were taken (Agilent In-Line UV Spectrophotometer), to obtain three spectra that were indistinguishable from the spectrum 100 shown in FIG. 2. The spectra all included a peak at 227 nm indicative of the taxane core structure in the paclitaxel, indicating that the paclitaxel solid forms of Example 1 were not distinguishable from each other based on UV spectra of the paclitaxel in solution.

### Example 3

### Infrared Spectra of Bulk Paclitaxel Samples

FTIR Infrared spectra each of the samples prepared in Example 1 were obtained following procedure: a pellet of KBr was made by grinding the paclitaxel crystal with KBr using a mortar and pestle at room temperature (about 23° C.); the resulting solid was placed under vacuum to remove residual methanol solvent (0.025 mmHg); and a spectra was recorded of the paclitaxel analyte. Representative spectra of each solid form of paclitaxel are provided in FIGS. 3A-3C, as discussed above. Infrared spectra may also be obtained using Attenuated Total Reflection Infrared (ATR-IR) from a coating or a small sample of a solid taxane sample from a coating. One suitable ATR-IR apparatus is the PerkinElmer Horizontal ATR model L1200361.

### Example 4

### Ultrasonic Spray Coating of Stents with Paclitaxel

Stents with coatings consisting of paclitaxel taxane therapeutic agent coatings including both the dihydrate solid form and in the amorphous solid forms of paclitaxel were prepared by spray coating a solution comprising paclitaxel, methanol and water. A paclitaxel solution in methanol and water was prepared. Specifically, a 1.74 mM paclitaxel solution was prepared in 68% methanol by dissolving 7.43 mg of paclitaxel in 5 mL of previously made solution of 68% methanol 32% water. The solution was sprayed from an ultrasonic spray gun (Sono-tek Model 06-04372) in a glove box. Before spraying, the glove box was purged with nitrogen at 20 psi for 15 minutes. The atmosphere in the glove box was adjusted until the oxygen meter reads a constant 200 ppm within the glove box. The heat in the glovebox was set to 31° C. (88° F.), the air shroud to 2.0 psi and the ultrasonic power to 1.0 W. The paclitaxel solution was loaded into a syringe and place on the syringe pump in the ultrasonic coating apparatus and a bare metal stent (6×20 ZILVER, Cook Inc., Bloomington, Ind.) was mounted on a mandrel aligned with the spray nozzle. The solution was sprayed onto a stent using a 60 kHz nozzle at a flow rate of 0.03 mL/min, a coating velocity of 0.025 in/sec, a nozzle power of 1.0 W, a process gas pressure of 2.0 psi, and a distance from the nozzle to the stent of about 12 mm, while rotating the stent with an axial rotation rate of 60 rpm. Only the abluminal surface of the stent was coated.

### Example 5

### Stents Coated with Single Layer of Therapeutic Agent

Anhydrous paclitaxel was applied to Zilver® stents (nitinol stents manufactured by Cook Inc., Bloomington, Ind.) ranging in size from 6×20 mm to 14×80 mm, as follows. First, paclitaxel was dissolved in ethanol to form a 2.4 mM solution. The paclitaxel was substantially dissolved within about 30 minutes, using sonication. The paclitaxel solution was then filtered through a 0.2 micron nylon filter and collected in a flask. Approximately 10 ml of ethanol was filtered through a 0.2 micron nylon filtered and then transferred into a reservoir connected to a spray gun nozzle. This solution was then used to set the flow rate of the spray gun to the target flow rate of approximately 5.7 ml/min. Stents were mounted on a mandrel assembly positioned in the lumen of the stent, including a silicon tube covering a steel rod. This assembly masked the lumens of the stents and substantially prevented the lumens from being coated.

Approximately 25 ml of the filtered paclitaxel solution was added to the spray gun reservoir, and the solution was sprayed onto the stents using a conventional pressure spray gun manufactured by Badger (Model No. 200), in a HEPA filtered hood, with a fluid dispensing system connected to a pressure source (nitrogen) until the target dose of paclitaxel was reached. Adjustments on the system were used to control the spray pattern and the amount of fluid dispensed. The spray gun was aligned with the stents by setting a laser beam even with the nozzle of the spray gun and positioning the stents so that the laser beam was located at approximately ¼ the distance from the top of the stents. The spray gun, which was positioned parallel to the hood floor and at a horizontal distance of approximately 5-7 inches from the stents, was then passed over the surface of the stents until a predetermined volume of spray was dispensed. The stents were then rotated approximately 90 degrees and the spraying procedure were repeated until the entire circumference of each stent was coated. The movement of the gun was slow enough to allow the solvent to evaporate before the next pass of the gun. Each spray application covered approximately 90 degrees of the circumference of the stents. The stents were kept at ambient temperature and humidity during the spraying process. After substantially all of the solvent had evaporated, a coating of paclitaxel was left on the stent.

### Example 6

### Elution of Paclitaxel-Coated Stents in Porcine Serum

Stents with coatings consisting of paclitaxel taxane therapeutic agents in both the dihydrate solid form and in the amorphous form were prepared by spray coating a solution comprising various amounts of paclitaxel, methanol and water. A 2.34 mM paclitaxel solution in 88% methanol and 12% water (v) was made with a total volume of about 10 mL (20.04 mg paclitaxel). Twelve (12) 6×20 ZILVER (Cook Inc., Bloomington, Ind.) stents were spray coated using the ultrasonic coating procedure of Example 5 and the parameters in Table 9 below. Table 10 also shows the amount of paclitaxel coated on each stent.

TABLE 9
Coating Parameters for Stents Coated with 2.34 mM Paclitaxel

| | Coating Solution | | | |
|---|---|---|---|---|
| | 2.34 mM PTX in 88% MeOH/H₂O | | | |
| | Stents | | | |
| | 1-3 | 4-6 | 7-9 | 10-12 |
| Relative Humidity (%) | 8.7-13.3 | 7.3-8.5 | 7.1-8.3 | 7.4-8.2 |
| Temperature (degrees F.) | 82.4-83.1 | 83.1 | 83.3-83.4 | 83.7-84.0 |

| | |
|---|---|
| Target Dose (micrograms) | 74 |
| Actual Dose (micrograms) | 84 ± 5.89 |
| Flow Rate (mL/min) | 0.03 |
| Loops | 5 |
| Air Shroud (psi) | 1.0 |
| Linear Velocity (in/sec) | 0.025 |
| Rotational Velocity (rpm) | 60 |
| Oxygen Content (ppm) | 145-155 |
| Power (Watts) | 0.8 |
| Nozzle Distance from | 8 |
| Stent (mm) | |

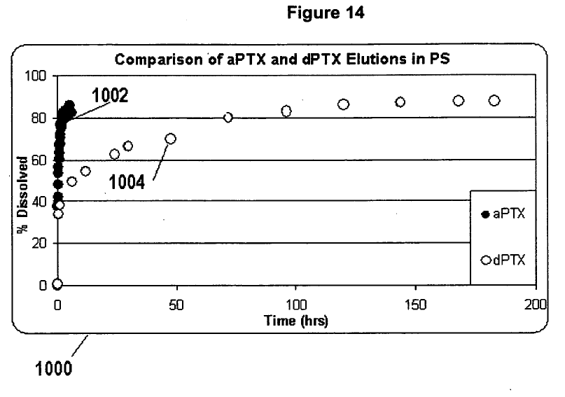

FIG. 14 shows an elution graph 1000 comparing a first elution profile 1002 for a 100% amorphous paclitaxel coating (formed by spray coating an ethanol-paclitaxel according to Example 4B) compared to a second elution profile 1004 obtained as the average of the 12 stent coatings according to Table 9 (containing about 50% dihydrate paclitaxel) (both in porcine serum). Increasing the amount of dihydrate resulted in sustained release of the paclitaxel in the second elution profile 1004 compared to the first elution profile 1002. FIG. 14 was obtained from a coated vascular stent having an amorphous paclitaxel (1002) or a 50% dihydrate:50% amorphous paclitaxel coating (1004) obtained in separate experiments during the continuous flow of a porcine serum elution medium. The coatings did not comprise a polymer. The amount of paclitaxel in the elution medium was measured by UV absorption at 227 nm. The first elution profile 1002 shows substantially all of the amorphous paclitaxel eluting within less than about 5 hours. The second elution profile 1004 in porcine serum elution medium showed about 60% of the paclitaxel coating eluted after about 25 hours and about 80% of the paclitaxel coating etuted from the coating after 75 hours.

### Example 7

### Elution of Paclitaxel-Coated Stents in HCD

Stents with coatings consisting of paclitaxel taxane therapeutic agents in both the dihydrate solid form and in the amorphous form were prepared by spray coating a solution comprising various amounts of paclitaxel, methanol and water. First, a first coating solution of 4.68 mM paclitaxel solution in 100% ethanol was prepared with 19.96 mg paclitaxel in 5 mL ethanol. Second, a second solution of 4.68 mM paclitaxel in 93% methanol and 7% water (v) was made with a total volume of about 5 mL (19.99 mg paclitaxel). Five (5) 6×20 ZILVER (Cook Inc., Bloomington, Ind.) stents were spray coated with the first spray solution and five (5) more 6×20 ZILVER (Cook Inc., Bloomington, Ind.) stents were spray coated with the second spray solution. All coating was performed on the abluminal surface only using the ultrasonic coating procedure of Example 5 and the parameters in Table 10 below. Table 10 also shows the amount of paclitaxel coated on each stent. Coatings formed from the first solution (ethanol) contained 93% amorphous paclitaxel, 7% dihydrate paclitaxel; coatings formed from the second solution (methanol/water) contained about 82% dihydrate and 18% amorphous paclitaxel.

TABLE 10
Coating Parameters for Stents Coated with 4.68 mM Paclitaxel

| | Coating Solvent | | | |
|---|---|---|---|---|
| | EtOH | | 93% MeOH/H₂O | |
| | Stent #s | | | |
| | 100-102 | 103-105 | 200-202 | 203-205 |
| Temperature (degrees F.) | 79.2 | 79.4-79.5 | 78.3-79.0 | 77.2-78.1 |
| Oxygen Content (ppm) | 135-165 | 125-145 | 135-145 | 135-180 |

| | | | |
|---|---|---|---|
| Relative Humidity (%} | 0.0 | 0.0-0.8 | 0.0 |

| | | |
|---|---|---|
| Power (Watts) | 1.1 | 0.8 |
| Actual Dose (μg) | 195 ± 17 | 301 ± 10 |

| | |
|---|---|
| Flow Rate (mL/min) | 0.03 |
| Loops | 7 |
| Air Shroud (psi) | 1.0 |
| Linear Velocity (in/sec) | 0.025 |
| Rotational Velocity (rpm) | 60 |
| Nozzle Distance from | 8 |
| Stent (mm) | |
| Target Dose (μg) | 219 |

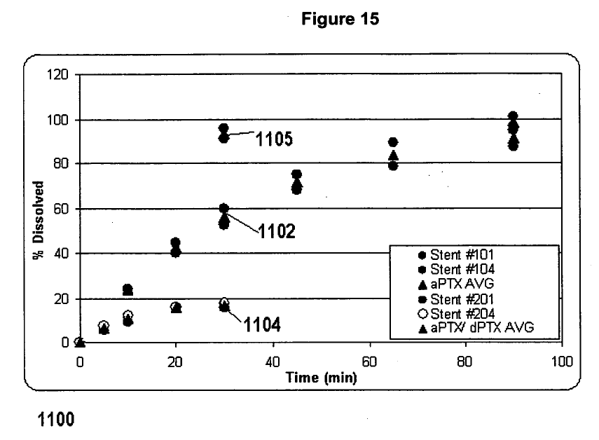

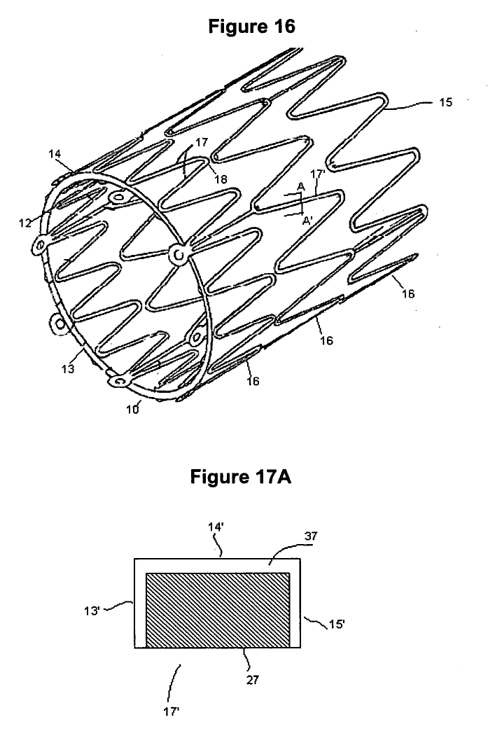

FIG. 15 shows an elution graph 1100 obtained in a 0.5% aqueous HCD solution, comparing a first elution profile 1102 from the coatings formed from the 93% amorphous paclitaxel coating deposited from the first solution (formed by ultrasonic spray coating an according to Example 5, except as indicated in Example 7) compared to a second elution profile 1104 obtained from the stent coatings from the 82% dihydrate coating deposited from the second solution (formed by ultrasonic spray coating an according to Example 5, except as indicated in Example 7). The coatings did not comprise a polymer. The amount of paclitaxel in the elution medium was measured by UV absorption at 227 nm. The first elution profile 1102 shows a more rapid elution rate than the second elution profile 1104. Data points 1105 were obtained by contacting the coated stent formed from the second solution with 100% ethanol after obtaining the second elution profile 1104, resulting in rapid release of all remaining paclitaxel from the coating.

### Example 8

### Single Layer of PLA Over Single Layer of Paclitaxel on a Stent Using Pressure Gun Spray Coating Method

Anhydrous paclitaxel was applied to Zilver® stents (nitinol stents manufactured by Cook Inc., Bloomington, Ind.) ranging in size from 6×20 mm to 14×80 mm, as follows. First, a layer of paclitaxel was applied as described in Example 5.

After the paclitaxel layer air dried, a layer PLA was then spray deposited over the paclitaxel coating using the same type of pressure spray coating apparatus as Example 5. A solution of approximately 2-4 g/L of PLA in dichloromethane was prepared, filtered over a 0.2 micron nylon filter, and collected in a flask. The solution was then sprayed over the coating of paclitaxel using a procedure similar to the one described above with respect to paclitaxel. For PLA, however, the spraying is performed at two different heights. First, the stents were positioned approximately 115 mm from the hood floor, sprayed, and rotated until the circumference of the top portion of the stents was coated. Next, the stents were positioned approximately 130 mm from the hood floor, sprayed, and rotated until the circumference of the bottom portion of the stents was coated.

Three different stent systems were tested: a vascular stent having a first layer of 69 μg paclitaxel deposited on the abluminal surface of the stent, and 173 μg of poly(D,L)lactic acid deposited in a second layer over the paclitaxel; a two-layer coating having a first layer of 5 μg paclitaxel deposited on the abluminal surface of the stent, and 73 μg of poly(D,L)lactic acid deposited in a second layer over the paclitaxel; and a two-layer coating having a first layer of 69 μg paclitaxel deposited on the abluminal surface of the stent, and 88 μg of poly(D,L)lactic acid deposited in a second layer over the paclitaxel. Numerical data for some of the resulting coated stents (obtained using a UV detection of paclitaxel in the modified porcine serum elution assay described Example 7) are shown below in Table 11.

TABLE 11

| | % PTX Dissolved | | |
|---|---|---|---|
| Time | 69 μg PTX/ | 5 μg PTX/ | 69 μg PTX/ |
| (hrs) | 173 μg PLA | 73 μg PLA | 88 μg PLA |
| 0 | 0.00 | 0.00 | 0.00 |
| 6 | 39 | 28 | 56 |
| 12 | 48 | 33 | 65 |
| 24 | 53 | 37 | 74 |
| 30 | 56 | 40 | 76 |
| 46 | 59 | 44 | 79 |
| 68 | 61 | 49 | 82 |
| 90 | 62 | 53 | 84 |
| 110 | 63 | 54 | 85 |
| 113 | 63 | 54 | 86 |
| 132 | 64 | 56 | 87 |
| 154 | 65 | 58 | 87 |
| 175 | 66 | 59 | 88 |
| 176 | 66 | 59 | 88 |
| 197 | 67 | 61 | 88 |
| 221 | 68 | 63 | 89 |
| 243 | 69 | 64 | 89 |
| 289 | 70 | 66 | 89 |
| 329 | 70 | 67 | 89 |
| 375 | 71 | 68 | 89 |
| 393 | 71 | 69 | 89 |
| 415 | 71 | N/A | 90 |
| 461 | 72 | 70 | 90 |
| 480 | 72 | 70 | 90 |
| 507 | 72 | 71 | 90 |

### Example 9

### Porcine Serum Assay to Measure Paclitaxel Elution from a Coated Vascular Stent

Porcine serum (1500 mL) was thawed in a water bath at 37° C. Once the porcine serum was thawed, heparin was added to avoid coagulation. 0.104 mL of a 6 g/L Heparin solution in water is added per mL of porcine serum. The pH of the media is regulated using an aqueous solution of acetic acid (20% v/v). The acidic solution is added to the porcine serum until the pH meter indicates a pH of 5.6±0.3. The initial and final temperature and the initial and final pH are recorded. Once the porcine serum is ready, 7-250 mL Erlenmeyer flasks are filled with 202.00±0.05 g. A stir bar should be placed in each flask and the lids are placed on the corresponding Erlenmeyer flask. The flask corresponding to the violet chamber, which is the control channel, is spiked with 10 μL of an ethanolic 1.2 mM PTX solution.

The 250 mL Erlenmeyer flasks are placed on the 10-well stir plate and it is ensured that the solutions are being stirred. The inlet and outlet tubes are placed into appropriate places in the flask. The stents are placed in the corresponding channel. The cells are assembled. After setting the time points, the cells are inserted and the test is started and allowed to run for the established period of time. A 4 L beaker with DiW and a lint free cloth is placed into the water to clean the auto-sampler head after the sample is collected. 4-mL samples are collected and sent to a UV-VIS spectrophotometer (or other suitable detector) to detect the presence of the therapeutic agent (e.g., paclitaxel absorption at 227 nm), or transferred to a cryovial tube and placed in the freezer at −25° C., and then shipped on dry ice for later analysis.

### Example 10

### Single Layer of Zein Over Single Layer of Paclitaxel on a Stent

Amorphous paclitaxel was applied to several 6×20 mm Zilver® stents (nitinol stents manufactured by Cook Inc.) as follows. First, paclitaxel was dissolved in ethanol to form a 2.4 mM solution. The paclitaxel was substantially dissolved within about 30 minutes, using sonication. The paclitaxel solution was then filtered through a 0.2 micron nylon filter and collected in a flask.

Approximately 10 mL+/−0.1 mL of ethanol was filtered through a 0.2 micron nylon filter and then transferred into a reservoir connected to a pressure spray gun nozzle. This solution was then used to set the flow rate of the pressure spray gun to the target flow rate of approximately 5.7 mL/min.+/−mL/min.

Some stents were mounted on a mandrel assembly positioned in the lumen of the stent, including a silicon tube covering a steel rod. This assembly masked the lumens of the stents and substantially prevented the lumens from being coated.

Approximately 25 mL of the filtered paclitaxel solution was added to the spray gun reservoir, and the solution was sprayed onto the stents using a HEPA filtered hood and a fluid dispensing system connected to a pressure source (nitrogen) until the target dose of paclitaxel was reached (for comparison, some stents were coated with more paclitaxel than others). Adjustments on the system were used to control the spray pattern and the amount of fluid dispensed. The spray gun was aligned with the stents by setting a laser beam even with the nozzle of the spray gun and positioning the stents so that the laser beam was located at approximately ¼ the distance from the top of the stents. The spray gun, which was positioned parallel to the hood floor and at a horizontal distance of approximately 12-18 centimeters from the stents, was then passed over the surface of the stents until a predetermined volume of spray was dispensed. The stents were then rotated approximately 90 degrees and the spraying procedure repeated until the entire circumference of each stent was coated. The movement of the gun was slow enough to allow the solvent to evaporate before the next pass of the gun. Each spray application covered approximately 90 degrees of the circumference of the stents. The stents were kept at ambient temperature and humidity during the spraying process, and the solution was pumped at a rate of approximately 6 mL/min through the pressure spray gun. After substantially all of the solvent had evaporated, a coating of paclitaxel between about 0.07 μg mm-2 and about 1.37 μg mm-2 was left on the stent.

Zein was then applied over the paclitaxel coating. A solution of approximately 2 g/L of zein in methanol was prepared, filtered over a 0.2 micron nylon filter, and collected in a flask. The Methanolic solution of zein was then deposited over the layer of paclitaxel using an ultrasonic nozzle. The ultrasonic nozzle power was about 1.1 watts with a flow rate between 0.06 mL/min. and 0.08 mL/min. The nozzle was positioned at a horizontal distance of between approximately 11 mm and 15 mm from the stents. The zein solution was coated on the stent at a velocity of about 25.5 mm/sec.

The coated stent was sterilized with ethylene oxide, and loaded into a flask containing HCD. Samples were taken at intervals and analyzed for paclitaxel. Numerical data for some of the resulting coated stents is shown in tables 12 and 13 below.

TABLE 12
68 μg PTX/69 μg Zein

| Time (min) | % PTX Eluted |
|---|---|
| 0 | 0 |
| 3 | 2 |
| 8 | 18 |
| 11 | 23 |
| 14.5 | 26 |
| 37 | 45 |
| 64 | 58 |
| 90 | 58 |
| 144 | 65 |
| 199 | 69 |
| 297 | 69 |
| 434 | 70 |

TABLE 13
79 μg PTX/149 μg Zein

| Time (min) | % PTX Eluted |
|---|---|
| 0 | 0 |
| 3 | 5 |
| 8 | 16 |
| 23 | 34 |
| 44 | 47 |
| 69 | 54 |
| 123 | 57 |
| 180 | 59 |
| 273 | 67 |
| 349 | 68 |
| 405 | 69 |

Although exemplary embodiments of the invention have been described with respect to the treatment of complications such as restenosis following an angioplasty procedure, the local delivery of therapeutic agents may be used to treat a wide variety of conditions using any medical device.

We claim:

1. A method of detecting a taxane therapeutic agent in a medical device coating, the method comprising the steps of:
a. contacting the coated medical device with an elution medium comprising a cyclodextrin; and
b. detecting the taxane therapeutic agent in the elution medium.

2. The method of claim 1, wherein the cyclodextrin comprises Heptakis-(2,6-di-O-methyl)-β-cyclodextrin.

3. The method of claim 1, wherein the taxane therapeutic agent comprises paclitaxel.

4. The method of claim 1, wherein the elution medium is an aqueous solution comprising between about 0.1% and 10% by volume of the cyclodextrin.

5. The method of claim 4, wherein the step of contacting the coated medical device with the elution medium comprises positioning the coated medical device in a fluid stream of the elution medium.

6. The method of claim 1, wherein the coating comprises a release modifying agent and a taxane therapeutic agent.

7. The method of claim 6, wherein the coating comprises a first layer comprising paclitaxel positioned between a surface of the coated medical device and a second layer comprising a bioabsorbable elastomer.

8. The method of claim 6, wherein the release modifying agent is selected from the group consisting of: PLA, PGA, PLGA and zein.

9. The method of claim 1, wherein the method further comprises the steps of detecting the presence of the taxane therapeutic agent in the elution medium over a first time period, and generating an elution profile from the amount of taxane therapeutic agent detected in the elution medium during the first period.

10. The method of claim 1, wherein the coating does not contain a release modifying agent.

11. The method of claim 1, wherein the coating comprises a taxane therapeutic agent in a first taxane solid form characterized by a vibrational spectrum comprising at least two peaks between 1740 and 1700 cm⁻¹ and having a solubility of less than 40% wt. after 1 hour in a 0.5% aqueous solution of Heptakis-(2,6-di-O-methyl)-β-cyclodextrin at 25° C.

12. The medical device of claim 11, wherein the first solid form of the taxane therapeutic agent has a melting point of between about 210 and 215° C.

13. The medical device of claim 11, wherein the coating further comprises a second taxane solid form of the taxane therapeutic agent characterized by a vibrational spectrum comprising a single peak between 1740 and 1700 cm⁻¹ and a solubility of greater than 50% wt. after 1 hour in a 0.5% aqueous solution of Heptakis-(2,6-di-O-methyl)-β-cyclodextrin at 25° C.

14. A lot release testing method comprising the steps of:
a. coating a medical device with a taxane therapeutic agent to form a standard coated medical device in compliance with at least one lot testing criterion;
b. contacting the standard coated medical device with a first elution medium comprising a cyclodextrin for a first period of time;
c. measuring the taxane therapeutic agent in the first elution medium as a function of time the standard coated medical device is in contact with the elution medium to obtain a standard elution profile;
d. selecting a sample coated medical device including a taxane therapeutic agent from a first lot of coated medical devices;
e. contacting the sample coated medical device with a second elution medium comprising a cyclodextrin for a second period of time;
f. measuring the taxane therapeutic agent in the second elution medium as a function of time the sample coated medical device is in contact with the elution medium to obtain a sample elution profile;
g. comparing the first elution profile with the second elution profile to determine whether the sample coated medical device meets the at least one lot testing criterion.

15. The lot release testing method of claim 14, wherein the first period of time is substantially equal to the second period of time and is less than about 12 hours.

16. The lot release testing method of claim 14, wherein the first elution medium and the second elution medium each comprise an aqueous solution comprising between about 0.1% and 10% Heptakis-(2,6-di-O-methyl)-β-cyclodextrin.

17. The lot release testing method of claim 14, further comprising the steps of:
a. contacting the standard coated medical device with a third elution comprising sodium dodecyl sulfate after contacting the standard medical device with the first elution medium comprising a cyclodextrin;
b. detecting the taxane therapeutic agent in the third elution medium;
c. contacting the sample coated medical device with a fourth elution comprising sodium dodecyl sulfate after contacting the standard medical device with the second elution medium comprising a cyclodextrin; and
d. detecting the taxane therapeutic agent in the fourth elution medium.

18. The lot release method of claim 14, comprising the steps of:
a. coating a medical device with paclitaxel to form a standard coated medical device in compliance with at least one lot testing criterion;
b. contacting the standard coated medical device with a first elution medium comprising 0.2-0.5% HCD cyclodextrin for a first period of time;
c. measuring the taxane therapeutic agent in the first elution medium as a function of time the standard coated medical device is in contact with the elution medium to obtain a standard elution profile;
d. contacting the standard coated medical device with a third elution comprising sodium dodecyl sulfate after contacting the standard medical device with the first elution medium comprising a cyclodextrin;
e. detecting the taxane therapeutic agent in the third elution medium;
f. selecting a sample coated medical device including a taxane therapeutic agent from a first lot of coated medical devices;
g. contacting the sample coated medical device with a second elution medium comprising 0.2-0.5% HCD cyclodextrin for a second period of time;
h. measuring the taxane therapeutic agent in the second elution medium as a function of time the sample coated medical device is in contact with the elution medium to obtain a sample elution profile;
i. contacting the sample coated medical device with a fourth elution comprising sodium dodecyl sulfate after contacting the standard medical device with the second elution medium comprising a cyclodextrin; and
j. detecting the taxane therapeutic agent in the fourth elution medium.
k. comparing the first elution profile with the second elution profile to determine whether the sample coated medical device meets the at least one lot testing criterion.

19. The lot release method of claim 18, wherein the first period of time and the second period of time are independently between about 1 hour and 8 hours, and wherein the standard coated medical device coating is free of a polymer.

20. A lot release testing method, comprising the steps of:
a. providing a first coated medical device comprising paclitaxel in a solid form characterized by a vibrational spectrum comprising at least two peaks between 1740 and 1700 cm⁻¹ and having a solubility of less than 40% wt. after 1 hour in a 0.5% aqueous solution of Heptakis-(2,6-di-O-methyl)-β-cyclodextrin at 25° C.;
b. contacting the first coated medical device with a first elution medium comprising an aqueous solution comprising between about 0.1% and 10% Heptakis-(2,6-di-O-methyl)-β-cyclodextrin for a time period effective to elute the paclitaxel from the medical device;
c. detecting the paclitaxel in the first elution medium by detecting the UV absorption of the paclitaxel in the first elution medium at about 227 nm;
d. recording a first elution profile of the paclitaxel from the first coated medical device in the first elution medium based on the paclitaxel detected in the first elution medium;
e. providing a second coated medical device comprising paclitaxel;
f. contacting the second coated medical device with the first elution medium;
g. detecting the paclitaxel in the first elution medium by detecting the UV absorption of the paclitaxel in the first elution medium at about 227 nm;
h. recording a second elution profile of the paclitaxel from the second coated medical device in the first elution medium based on the paclitaxel detected in the first elution medium;
i. contacting the first coated medical device with a second elution medium comprising ethanol or sodium dodecyl sulfate for a period of time effective to elute the paclitaxel from the medical device;
j. detecting the paclitaxel in the second elution medium; and
k. recording an elution profile of the paclitaxel from the second medical device in the second elution medium based on the amount of paclitaxel detected in the second elution medium.
